(19) United States
(12) Patent Application Publication
(10) Pub. No.: US 2026/0317370 A1
(43) Pub. Date: Oct. 15, 2026

(54) METHOD OF DEPROTECTING AT LEAST ONE D-AMINO ACID AND/OR D-AMINO ACID DERIVATIVE PROTECTED FUNCTIONAL GROUP

(71) Applicant: Martin-Luther-Universität Halle-Wittenberg, Halle (Saale) (DE)

(72) Inventors: Andreas Hans Simon, Halle (DE); Sandra Liebscher, Halle (DE); Frank Bordusa, Rossbach (DE)

(21) Appl. No.: 19/481,273

(22) PCT Filed: May 6, 2024
(86) PCT No.: PCT/EP2024/062484

§ 371 Date: Nov. 4, 2025

(30) Foreign Application Priority Data
May 4, 2023 (EP) 23171567.3

(51) Int. Cl. C07K 1/12; C07K 1/04; C12N 9/48

(52) U.S. Cl. CPC C07K 1/12; C07K 1/042; C12N 9/485; C12Y 304/11019

(57) ABSTRACT

The present invention relates, to a method of deprotecting at least one D-amino acid and/or D-amino acid derivative-protected functional group of at least one substrate having at least one D-amino acid and/or D-amino acid derivative-protected functional group, to a D-stereospecific hydrolytic enzyme, and to the use of this D-stereospecific hydrolytic enzyme. In addition, the present invention relates to a method of manufacturing a compound that includes the method of deprotecting and to the use of a D-amino acid and/or D-amino acid derivative as a protecting group for a functional group of a substrate.

### FIELD OF THE INVENTION

The present invention relates, to a method of deprotecting at least one D-amino acid and/or D-amino acid derivative-protected functional group of at least one substrate having at least one D-amino acid and/or D-amino acid derivative-protected functional group, to a D-stereospecific hydrolytic enzyme, and to the use of this D-stereospecific hydrolytic enzyme. In addition, the present invention relates to a method of manufacturing a compound that includes the method of deprotecting and to the use of a D-amino acid and/or D-amino acid derivative as a protecting group for a functional group of a substrate. In particular, the present invention enables the selective hydrolytically cleavage of D-amino acids and/or D-amino acid derivatives from mono-, bi- and multifunctional substrates using a D-stereospecific hydrolytic enzyme.

The invention further allows a new protective group strategy and the selective cleavage of affinity- or solubility-mediating functionalities in organic synthesis.

### BACKGROUND OF THE INVENTION

In order to guarantee a selective reaction of two functional groups in a chemical synthesis of, in particular bi- or multifunctional, compounds, it is usually necessary to temporarily protect the additional reactive groups present in the reactants.

Unprotected functionalities with comparable or similar reactivity, on the other hand, lead to undesired side reactions, which in turn inevitably result in yield losses and the formation of heterogeneous product mixtures that generally require a costly purification step after the reaction has taken place.

Already established chemical processes for protecting and, in particular, deprotecting such functionalities often require reaction conditions that do not reliably guarantee the integrity of the reactants and/or the products and are therefore unsuitable in particular (but not exclusively) for labile target structures. Furthermore, particularly multi-step synthesis requires the use of a highly orthogonal protecting group strategy, as otherwise an accumulation of undesired by-products in each individual reaction step is to be expected, preventing high synthesis yields even at a few reaction steps.

One deprotecting strategy that has already been prevailed includes the use of L-amino acids as protecting agents that can be enzymatically cleaved. However, since L-amino acids are widely used in organic synthesis, the deprotecting, i.e. the cleavage of the L-amino acid, is not very selective which leads to the above mentioned problems.

E. Barbayianni et al. (E. Barbayianni, I. Fotakopoulou, M. Schmidt, V. Constantinou-Kokotou, U. T. Bornscheuer, G. Kokotos G, “Enzymatic Removal of Carboxyl Protecting Groups. 2nd Cleavage of the Benzyl and Methyl Moieties”, The Journal of Organic Chemistry, 2005, 70, 22, pp. 8730-8733, doi: 10.1021/jo051004v) describe hydrolases such as the esterase BS2 from Bacillus subtilis and various lipases such as CAL-A from Candida antarctica for the selective hydrolysis of ester bonds, which are used as protective groups of carboxylic acids and hydroxy groups. Similarly, F. Bellezza et al. (F. Bellezza, A. Cipiciani, G. Cruciani, F. Fringuelli, “The importance of ester and alkoxy type functionalities for the chemo- and enantio-recognition of substrates by hydrolysis with Candida rugosa lipase”, Journal of the Chemical Society, Perkin Transactions, 2000, 1, 24, pp. 4439-4444, doi: 10.1039/B005512N) describe such strategy using lipases such as CAL-A from Candida rugose. Furthermore, H. Waldmann et al. (H. Waldmann, A. Reidel, “The phenylacetyl group—the first enzymatically cleavable amino-protecting group in solution and on solid phase”, Applied Chemistry, 1997, 109, 6, pp. 642-644, doi: 10.1002/ange.19971090620) disclose that amino functions can be masked by phenylacetic acid, and the formed phenylacetic acid amide can be selectively cleaved again by the enzyme penicillin G acylase.

Recent studies from M. Reille-Seroussi et al. (M. Reille-Seroussi, S. V. Mayer, W. Dörner, K. Lang, H. D. Mootz, “Expanding the genetic code with a lysine derivative bearing an enzymatically removable phenylacetyl group”, Chemical Communications, 2019, 55, pp. 4793-4796, doi: 10.1039/C9CC00475K) show that these phenylacetyl functionalities can also be selectively removed from lysine side chains by selected sirtuins. In addition, A. G. Gum et al. (A. G. Gum, T. Kappes-Roth, H. Waldmann, “Labile Protecting Groups in Peptide Synthesis: Development of Glucose- and Galactose-Derived Urethanes”. Chemistry—A European Journal, 2000, 20, pp. 3714-3721, doi: 10.1002/1521-3765(20001016)6:20<3714::AID-CHEM3714>3.0.CO; 2-Z) describe glucose- and galactose-based urethane protecting groups, which can be removed from selected functionalities by the use of glycosidases.

However, the methods above all have certain drawbacks.

For example, the enzyme penicillin G acylase, e.g. used for deprotecting amino functions, exhibits a very high KM value for the substrate used as a protecting group and thus, it shows only a low specific activity towards phenylacetic acid amides. Furthermore, the cleaved product leads to a very pronounced inhibition of the biocatalyst, which is accompanied by losses in yield as well as prolonged conversion rates, as investigated by W. B. L. Alkema et al. (W. B. L. Alkema, R. Floris, D. B Janssen, “The Use of Chromogenic Reference Substrates for the Kinetic Analysis of Penicillin Acylases”, Analytical Biochemistry, 1999, 275, 1, pp. 47-53, doi: https://doi.org/10.1006/abio.1999.4300). For these reasons, this enzyme is not used in preparative synthesis approaches.

The use of glucose- and galactose-based urethane protecting groups and their cleavage by glycosidases also does not find application on a preparative scale. The sugar-building components glucose and galactose contain reactive hydroxy groups, which must be fully protected during synthesis due to their chemical reactivity. However, these protected sugar derivatives, in turn, are not recognized by the glycosidases, which necessitates an additional reaction step, namely the removal of the protective groups from the actual protecting group. This, in turn, is accomplished by the use of lipases. This results in a very complex process, which has, therefore, not found any application in preparative synthesis.

Therefore, there is a need for new and highly selective deprotecting strategies of protected functional groups that require mild reaction conditions and that are compatible with all reactants and chemical structures present in preparative synthesis methods for manufacturing chemical compounds.

### SUMMARY OF THE INVENTION

The present invention exploits the use of D-stereospecific hydrolytic enzymes that selectively recognize D-amino acids and/or their derivatives (instead of the known L-amino acids) and hydrolytically cleave them from target structures, e.g., as a protecting group or functional tag. Since the reactivity of D-amino acids and/or D-amino acid derivatives under the achiral synthesis conditions of classical peptide chemistry does not differ in principle from that of its enantiomeric L-amino acid, these are compatible with already established synthesis strategies and, like the natural L-amino acids, can be easily introduced into target structures. The D-stereospecific hydrolytic enzymes used in the invention are stringently regio-, chemo- and enantioselective for the respective recognized D-amino acid and/or D-amino derivative. The cleavage occurs rapidly and quantitatively, without side reactions, under mild reaction conditions, and is orthogonal to all previously established deprotecting and synthesis methods.

The invention is concerned with the use of D-amino acids and/or D-amino acid derivatives to protect chemically reactive functional groups, such as amino, hydroxy, carboxylic acid, sulfhydryl and selenohydryl functionalities, and to deprotect them with the use of a D-stereospecific hydrolytic enzyme. An important aspect of the invention is the highly selective deprotecting of the D-amino acid and/or D-amino acid derivative-protected functional group(s) by a D-stereospecific hydrolytic enzyme that is substrate-specific for these D-amino acids and/or D-amino acid derivatives used as a protecting group. Since the deprotecting reactions are enzyme-catalyzed and subsequently occur under mild reaction conditions, this method can be fully applied even in the case of chemically labile substrate and target structures. Furthermore, the invention is orthogonal to all already established protecting group strategies, e.g. in peptide and carbohydrate chemistry, and expands the existing spectrum of methods, whereby complex multistep syntheses or sequential reaction sequences can also be realized, e.g. if they are required in preparative synthesis methods.

Moreover, the invention allows not only the selective deprotecting of reactive functionalities in the substrates or products, but also, in the same way, the cleavage of any functionality from the same, such as those with solubility-mediating properties or even affinity probes linked to the target structure via a D-amino acid and/or D-amino acid derivative specifically recognized by the hydrolase.

In a first aspect, the present invention relates to a method of deprotecting at least one D-amino acid and/or D-amino acid derivative protected functional group of at least one substrate, the at least one substrate having at least one D-amino acid and/or D-amino acid derivative protected functional group, using a D-stereospecific hydrolytic enzyme, the method comprising:
Providing the at least one substrate, the D-stereospecific hydrolytic enzyme, and at least one solvent;
Mixing the at least one substrate, the D-stereospecific hydrolytic enzyme and the at least one solvent to obtain a reaction mixture; and
Hydrolyzing the at least one D-amino acid and/or D-amino acid derivative protected functional group of the at least one substrate using the D-stereospecific hydrolytic enzyme in the reaction mixture to obtain a D-amino acid and/or a D-amino acid derivative and an at least partially deprotected substrate with at least one deprotected functional group deprotected from the D-amino acid and/or D-amino acid derivative.

In a second aspect, the invention relates to a D-stereospecific hydrolytic enzyme, characterized by the following features:
at least one first 3₁₀-helix-2 structure comprising an amino acid sequence SXXK;
at least one first α-helix structure comprising an amino acids sequence YSN; and
at least one first β-sheet structure comprising an amino acid sequence HXG; wherein X is an amino acid selected from the group consisting of proteinogenic amino acids;
wherein the first 3₁₀-helix-2 structure, the first α-helix structure and the first β-sheet structure are arranged in close proximity.

In a third aspect, the invention relates to a use of the D-stereospecific hydrolytic enzyme for deprotecting of at least one D-amino acid and/or D-amino acid derivative protected functional group of a substrate, preferably wherein the D-stereospecific hydrolytic enzyme is characterized by the following features:
at least one first 3₁₀-helix-2 structure comprising an amino acid sequence SXXK;
at least one first α-helix structure comprising an amino acids sequence YSN; and
at least one first β-sheet structure comprising an amino acid sequence HXG; wherein X is an amino acid selected from the group consisting of proteinogenic amino acids; wherein the first 3₁₀-helix-2 structure, the first α-helix structure and the first β-sheet structure are arranged in close proximity, further preferably the D-stereospecific hydrolytic enzyme having an amino acid sequence chosen from SEQ ID No. 1, No. 2, No. 3, No. 4, No. 5, No. 6, No. 7, No. 8, and No. 9, below.

In a fourth aspect, a method of manufacturing a carbohydrate; a carbohydrate derivative; a nucleotide; an oligonucleotide; a polynucleotide; a nucleotide derivative; an oligonucleotide derivative; a polynucleotide derivative; an amino acid; a peptide; an oligopeptide; a polypeptide; a protein; a peptide intermediate; a peptide derivative; an oligopeptide derivative; a polypeptide derivative; or mixtures thereof, is provided, the method comprising:
Providing a precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, at least one D-amino acid and/or D-amino acid derivative, at least one first solvent, optionally at least one second solvent, optionally at least one third solvent, a D-stereospecific hydrolytic enzyme, and at least one first reagent;
Mixing the precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, the at least one first solvent and the at least one D-amino acid and/or D-amino acid derivative to obtain a first reaction mixture;
Reacting the precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof and the at least one D-amino acid and/or D-amino acid derivative to obtain a protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof;
Optionally removing the at least one first solvent from the first reaction mixture; Optionally reacting the protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, in one or more further reaction steps with one or more further reagents to obtain a reacted protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof;
Mixing the protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, or the reacted protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, the at least one first reagent and optionally the at least one second solvent to obtain a second reaction mixture;
Reacting the protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, or the reacted protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof and the at least one first reagent to obtain a protected carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof;
Optionally removing the at least one second solvent from the second reaction mixture; Mixing the protected carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, optionally the at least one third solvent and the D-stereospecific hydrolytic enzyme to obtain a third reaction mixture; and
Deprotecting the at least one D-amino acid and/or D-amino acid derivative from the protected carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof to obtain the carbohydrate; the carbohydrate derivative; the nucleotide; the oligonucleotide; the polynucleotide; the nucleotide derivative; the oligonucleotide derivative; the polynucleotide derivative; the amino acid; the peptide; the oligopeptide; the polypeptide; the protein; the peptide intermediate; the peptide derivative; the oligopeptide derivative; the polypeptide derivative; or mixtures thereof.

In a fifth aspect, the invention relates to a use of a D-amino acid and/or D-amino acid derivative as a protecting group for a functional group of a substrate.

Further aspects and embodiments of the invention are disclosed in the dependent claims and can be taken from the following description, figures and examples without being limited thereto.

### DESCRIPTION OF THE DRAWINGS

The enclosed drawings should illustrate embodiments of the present invention and convey a further understanding thereof. In connection with the description, they serve as an explanation of the concepts and principles of the invention. Other embodiments and many of the stated advantages can be derived in relation to the drawings. The elements of the drawings are not necessarily to scale towards each other. Identical, functionally equivalent and acting equal features and components are denoted in the figures of the drawings with the same reference numbers, unless noted otherwise.

FIGS. 1 to 21 show results obtained in the present Examples.

### DETAILED DESCRIPTION OF THE PRESENT INVENTION

### Definitions

If not defined otherwise, technical and scientific terms used herein have the same meaning as is generally understood by a skilled person in the field of the invention.

All ranges disclosed herein are to be considered to be supplemented by the term “about”, unless clearly defined to the contrary or otherwise clear from the context. As used herein, qualifiers like “about,” “approximately,” and combinations and variations thereof, are intended to include not only the exact amount or value that they qualify, but also some slight deviations therefrom, which may be due to manufacturing tolerances, measurement error, wear and tear, stresses exerted on various parts, and combinations thereof, for example.

As used herein, the term “substantially” means that the subsequently described parameter, event, or circumstance completely occurs or that the subsequently described parameter, event, or circumstance occurs to a great extent or degree. For example, the term “substantially” means that the subsequently described parameter, event, or circumstance occurs at least 90% of the time, or at least 91%, or at least 92%, or at least 93%, or at least 94%, or at least 95%, or at least 96%, or at least 97%, or at least 98%, or at least 99%, of the time, or means that the dimension or measurement is within at least 90%, or at least 91%, or at least 92%, or at least 93%, or at least 94%, or at least 95%, or at least 96%, or at least 97%, or at least 98%, or at least 99%, of the referenced dimension or measurement.

The use of the term “at least one” or “one or more” will be understood to include one as well as any quantity of more than one. In addition, the use of the phrase “at least one of X, Y, and Z” will be understood to include X alone, Y alone, and Z alone, as well as any combination of X, Y, and Z.

The use of ordinal number terminology (i.e., “first”, “second”, “third”, “fourth”, etc.) is solely for the purpose of differentiating between two or more items and, unless explicitly stated otherwise, is not meant to imply any sequence or order or importance to one item over another or any order of addition.

All numbers or percentages relating to amounts of a substance within this application are given in wt. %, unless clearly defined to the contrary or otherwise clear from the context.

In this application, enzymes are proteins that act as biological catalysts by accelerating chemical reactions. The molecules upon which enzymes may act are called substrates.

A carbohydrate, also known as a saccharide, is a biochemical compound that has a structure that includes carbon (C), hydrogen (H) and oxygen (O) atoms. Saccharides include monosaccharides, disaccharides, oligosaccharides, and polysaccharides. A disaccharide is a saccharide formed when two monosaccharides are linked by a glycosidic bond. An oligosaccharide is a saccharide formed when a small number, typically three to ten, of monosaccharides are linked by glycosidic bonds. The oligosaccharides can have linear or branched structures. A polysaccharide is a saccharide containing a large number, at least 11, of monosaccharides that are linked by glycosidic bonds. The polysaccharides can have linear or branched structures.

A nucleotide includes a nucleoside and at least one phosphate. The nucleoside includes a nucleobase and a monosaccharide. The nucleobase can e.g. be selected from the group consisting of adenine, cytosine, guanine, thymine, uracil, and modifications thereof. According to certain embodiments, the monosaccharide can be selected from the group consisting of ribose and deoxyribose. The phosphates are, if more than one is present, usually sequentially arranged, connected via a P—O—P-bond. The nucleotide is not particularly limited. According to certain embodiments, the nucleotide can be selected from the group consisting of adenosine monophosphate (AMP), guanosine monophosphate (GMP), cytidine monophosphate (CMP), uridine monophosphate (UMP) and deoxythymidine monophosphate (dTMP).

The oligonucleotide is an oligomer formed by a small number, typically around 2 to 12, of nucleotides. The nucleotides are linked by a phosphodiester bond, in particular a 3′,5′-phosphodiester bond. The oligonucleotide usually has a linear structure. The nucleotide forming the oligonucleotide is not particularly limited and can be as described above.

The polynucleotide is an oligomer formed by a large number, at least 13, of nucleotides. The nucleotides are linked by a phosphodiester bond, in particular a 3′,5′-phosphodiester bond. The polynucleotide usually has a linear structure. The nucleotide is not particularly limited and can be as described above.

Amino acids are compounds having an amino group and having a carboxylic group.

A peptide is a short chain of at least 2 amino acids linked by peptide bonds. An oligopeptide is a short chain of 2 to 10 amino acids linked by peptide bonds. A polypeptide is a long chain of 11 to 100 amino acids linked by peptide bonds. A protein is a long chain of at least 101 amino acids linked by peptide bonds.

An α-helix is a common motif in the secondary structure of proteins, enzymes etc. and is a right hand-helix conformation in which every backbone N—H group hydrogen bonds to the backbone C═O group of the amino acid located four residues earlier along the protein sequence.

A 3₁₀-helix is a structure in which amino acids are arranged in a right-handed helical structure. Each amino acid corresponds to a 120° turn in the helix (i.e., the helix has three residues per turn, the N—H group of an amino acid forming a hydrogen bond with the C═O group of the amino acid three residues earlier.

A β-sheet is a common motif in the secondary structure of proteins, enzymes etc. and consists of beta strands (β-strands) connected laterally by at least two or three backbone hydrogen bonds, forming a generally twisted, pleated sheet. A β-strand is a stretch of polypeptide chain typically 3 to 10 amino acids long with backbone in an extended conformation.

The unit M within the present disclosure refers to mol/l, unless stated otherwise.

Before the invention is described in exemplary detail, it is to be understood that this invention is not limited to the particular component parts of the process steps of the methods described herein as such methods may vary. It is also to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. For example, the term “a” as used herein can be understood as one single entity or in the meaning of “one or more” entities. It is also to be understood that plural forms include singular and/or plural referents unless the context clearly dictates otherwise. It is moreover to be understood that, in case parameter ranges are given which are delimited by numeric values, the ranges are deemed to include these limitation values.

As used herein, the terms “comprises”, “comprising”, “includes”, “including”, “has”, “having” or any other variation thereof, are intended to cover a non-exclusive inclusion. For example, a process, method, article, or apparatus that comprises a list of elements is not necessarily limited to only those elements, but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In a first aspect, the present invention provides a method of deprotecting at least one D-amino acid and/or D-amino acid derivative-protected functional group of at least one substrate, the at least one substrate having at least one D-amino acid and/or D-amino acid derivative protected functional group, using a D-stereospecific hydrolytic enzyme, the method comprising:
Providing the at least one substrate, the D-stereospecific hydrolytic enzyme, and at least one solvent;
Mixing the at least one substrate, the D-stereospecific hydrolytic enzyme and the at least one solvent to obtain a reaction mixture; and
Hydrolyzing the at least one D-amino acid and/or D-amino acid derivative protected functional group of the at least one substrate using the D-stereospecific hydrolytic enzyme in the reaction mixture to obtain a D-amino acid and/or a D-amino acid derivative and an at least partially deprotected substrate with at least one deprotected functional group deprotected from the D-amino acid and/or D-amino acid.

In the method of the first aspect, the step of providing the different components, i.e. the at least one substrate, the D-stereospecific hydrolytic enzyme, and at least one solvent, is not particularly limited. For example, the components can be provided with or without carrying out at least one further step, e.g. a synthesis step, before providing the different components. Such a further step can be e.g. selected from the group consisting of a chemical reaction—e.g. in a chemical synthesis, removing and/or isolating at least one of the components, and/or adding at least one component.

The step of mixing the different components is not particularly limited. The order in which the various components can be added is not particularly limited. For example, the at least one substrate and the at least one solvent can be added and mixed first, and then the D-stereospecific hydrolytic enzyme can be added and mixed. However, this order can be changed as required.

The mixing is not particularly limited and any known mixing method can be used. Mixing can be carried out in a suitable reaction vessel, in which also the hydrolyzing can be carried out. The reaction vessel is not particularly limited and any known reaction vessel can be used, as long as it allows for the hydrolysis reaction to take place.

Hydrolysis is any chemical reaction in which a molecule of water cleaves one or more chemical bonds. Hydrolysis can be a substitution, an elimination, and a solvation reaction, in which water is the nucleophile. In the method of the first aspect, the hydrolysis is catalyzed by the D-stereospecific hydrolytic enzyme.

Here, the D-stereo-hydrolytic enzyme can catalyze the hydrolysis of a bond between a D-amino acid and/or D-amino acid derivative and a functional group of the at least one substrate protected by the D-amino acid and/or D-amino acid derivative. Thereby, a water molecule, that can be e.g. provided by the at least one solvent, is introduced. The water molecule can react with the at least one D-amino acid and/or D-amino acid derivative protected functional group of the at least one substrate to obtain a D-amino acid and/or a D-amino acid derivative and an at least partially deprotected substrate (or more than one at least partially deprotected substrates in case more than one substrate is provided) with at least one deprotected functional group deprotected from the D-amino acid and/or D-amino acid derivative.

The D-amino acid is not particularly limited as long as the D-amino acid includes at least one carboxylic group, at least one amino group, and has the D-configuration. According to certain embodiments, the D-amino acid is selected from the group consisting of D-alanine (D-ala), D-arginine (D-arg), D-asparagine (D-asn), D-aspartate (D-asp), D-cysteine (D-cys), D-glutamate (D-glu), D-glutamine (D-gln), D-histidine (D-his), D-isoleucine (D-ile), D-leucine (D-leu), D-lysine (D-lys), D-methionine (D-met), D-phenylalanine (D-phe), D-proline (D-pro), D-serine (D-ser), D-threonine (D-thr), D-tryptophan (D-trp), D-tyrosine (D-tyr), D-valine (D-val), D-homocysteine (D-hcy), D-homoserine (D-hse), D-norleucine (D-nle), D-norvaline (D-nva), D-ornithin (D-orn), D-penicillamine (D-pen), D-pyroglutamate (D-pglu), D-gamma glutamic acid (D-gglu), D-citrulline (D-cit), D-2-aminobutyric acid (D-aba), D-iso-aspartic acid (D-isoasp), D-selenocysteine (D-sec), D-selenomethionine, D-methionine sulfoxid, D-methionine sulfone, D-azido-lysine and the thioxo amino acids resulting thereof.

According to certain embodiments, the D-amino acid is selected from the group consisting of D-alanine (D-ala), D-arginine (D-arg), D-aspartate (D-asp), D-glutamate (D-glu), D-glutamine (D-gln), D-histidine (D-his), D-leucine (D-leu), D-lysine (D-lys), D-methionine (D-met), D-phenylalanine (D-phe), D-proline (D-pro), D-serine (D-ser), D-tryptophan (D-trp), D-tyrosine (D-tyr), and D-valine (D-val). According to certain embodiments, the D-amino acid is selected from the group consisting of D-alanine (D-ala), D-glutamine (D-gln), D-histidine (D-his), D-leucine (D-leu), D-methionine (D-met), D-phenylalanine (D-phe), D-serine (D-ser), D-tryptophan (D-trp), D-tyrosine (D-tyr), and D-valine (D-val). According to certain embodiments, the D-amino acid is selected from the group consisting of D-leucine (D-leu), D-methionine (D-met), D-phenylalanine (D-phe), D-tryptophan (D-trp), and D-tyrosine (D-tyr).

By using one of these D-amino acids, the D-stereospecific hydrolytic enzyme can recognize the D-amino acid more effectively and the deprotecting of D-amino acid protected functional group of a substrate can be performed more effectively.

The D-amino acid derivative can include one D-amino acid having one or more functionality, as exemplified below. The D-amino acid derivative can include a D-amino acid in form of a salt which is not particularly restricted. The D-amino acid derivative can also include one or more of a posttranslational modification, an artificial derivatisation, and combinations thereof. According to certain embodiments, the one or more functionality, posttranslational modification and/or artificial derivatisation can be present at the N-terminus and/or C-terminus and/or side chain of this D-amino acid.

In the D-amino acid derivate, the D-amino acid is not particularly limited as long as the D-amino acid includes at least one carboxylic, at least one amino group, and having the D-configuration. According to certain embodiments, the D-amino acid can be selected from the D-amino acids described above. According to certain embodiments, the one or more functionality, posttranslational modification and/or artificial derivatisation can be bonded to the D-amino acid via a bond selected from the group consisting of an ester bond and a carboxamide bond.

According to certain embodiments, the one or more functionality can be selected from the group consisting of a protecting functionality (protective group), a physicochemical functionality, an analytical/preparative functionality, e.g. a marker, a biological/therapeutical/medical functionality, a biochemical functionality, and combinations thereof. According to certain embodiments, the one or more functionality can be selected from the group consisting of a protecting functionality, a physicochemical functionality, and an analytical/preparative functionality. According to certain embodiments, the one or more protecting functionality, also referred to as blocking functionality, is derived from a compound that can be selected from the group consisting of allyl carbamate, 1,3-dioxane, 1,3-dithiane, 1,3-dithiolane,1-chloroethyl carbamate, 1-(4,4-dimethyl-2,6-dioxocyclohex-1-ylidene)ethyl, 2-(trimethylsilyl)-ethoxy-methyl acetal, 2,2,2-trichloroethyl carbonate, 2-alkyl-1,3-oxazoline, 2-methoxyethoxymethyl ether, 2-naphtylmethyl ether, sulfonamides such as 4-methoxybenzenesulfonamide, 4-methoxybenzylether, 9-fluorenylmethyl carbamate, acetamide, acetate, acetic acid ester, acetonide, allyl ether, benzaldehyde acetal, benzoate, benzoic acid ester, benzyl carbamate, benzyl ester, benzyl ether, benzylamine, benzylidene acetal, benzylidene amine, benzyloxy carbamate, benzyloxymethyl acetal, carbamate, carbonate, diethyl acetal, dimethyl acetal, di-tert-butyl dioxasilinane, ethoxyethyl acetal, ethylene gylcol acetal, formamide, methoxymethyl acetal, methoxymethyl aminal, methoxymethyl ether, methoxypropyl acetal, methyl carbamate, methyl ester, N-(4-methoxybenzyl)-indole, N,N-dimethylhydrazone, neopentyl glycol acetal, phthalimide, pivalic acid ester, propyl ester, p-toluenesulfonamide, succinic acid, tert-butyl ester, tert-butoxy carbamate, tert-butyl carbamate, tert-butyl ester, tert-butyl ether, tert-butyldimethylsilyl ether, tert-butyldiphenylsilyl ether, tetrahydropyranyl ether, triethylsilyl ether, trifluoroacetamide, triisopropyl ether, trimethylsilyl cyanohydrin, trimethylsilyl ether, triphenylmethyl amine, nitrophenyl carbamate, and any alkylation agents resulting in formylation, methylation, ethylation, propylation and the like. According to certain embodiments, the one or more protecting functionality is a protecting group selected from the group consisting of a benzoyl group (Bz), a fluorenylmethyloxycarbonyl group (Fmoc), a tert-butyloxycarbonyl group (Boc), an acetyl group (Ac), a succinic group (Suc), benzyloxycarbonyl group (Z), a 2-azidomethylbenzyl group (Ambz), a 2-aminobenzoic acid group (Abz), a biotin group, and mixtures thereof. By using a D-amino acid having one or more protecting functionality the D-amino acid can be modified so that reactions with other components or compounds present in the reaction mixture can be prevented. It allows the use of the D-amino acid in a broad variety of reaction conditions. In addition, it allows the use of chemical more labile D-amino acids, which can be modified with one or more of these protecting functionalities. Furthermore, chemical properties like the chemical stability, the solubility, the compatibility with other components in the reaction mixture, and physical properties of the substrate having the D-amino acid derivative protected functional group like the physical stability, the boiling point, the melting point, etc., can be adapted.

According to certain embodiments, the one or more physicochemical functionality can be selected from the group consisting of a dye, e.g. a fluorescent inorganic dye, a fluorescent organic dye, a nonfluorescent inorganic dye, or a nonfluorescent organic dye; and spin probes such as organic radicals. According to certain embodiments, the one or more physicochemical functionality can be selected from the group consisting of nitrophenols, aza dyes, Alexa Fluor™ dyes, dyes prepared by dinitrobenzylation (dnp), and the like. By using a D-amino acid having one or more physicochemical functionality, the physicochemical properties of the D-amino acid can be modified. It allows a more adequate characterization of the substrate having the D-amino acid derivative protected functional group. In particular, an optical characterization can be simplified.

According to certain embodiments, the one or more analytical/preparative functionality can include affinity fusions. According to certain embodiments, the one or more analytical/preparative functionality can be selected from the group consisting of biotin, desthiobiotin, Strepp-tag II, His-tag, c-Myc-tag and the like. By using a D-amino acid having one or more analytical/preparative functionality, the analytical/preparative application of the substrate having the D-amino acid derivative protected functional group can be simplified. In particular, the characterization of this substrate with various analytical methods like spectroscopic or chromatographic methods can be simplified. In addition, the properties of the substrate having the D-amino acid derivative protected functional can be adapted so that, for example, the purification of a reaction mixture including this substrate is enhanced.

According to certain embodiments, the one or more biological/therapeutical/medical functionality can include toxins targeting crucial intracellular pathways to induce cell death. According to certain embodiments, the one or more biological/therapeutical/medical functionality can be selected from the group consisting of calicheamycin, monomethyl aurestatines, emtansines, doxorubicin, SN-38 (7-ethyl-10-hydroxycamptothecin), and the like. By using a D-amino acid having one or more biological/therapeutical/medical functionality, the biological/therapeutical/medical properties of the substrate having the D-amino acid derivative protected functional group can be enhanced.

According to certain embodiments, the one or more biochemical functionality can be selected from the group consisting of pro-peptides, signal peptides, and the like. A pro-peptide is an inactive protein (or peptide) that can be turned into an active form by post-translational modification, such as cleaving a part of the molecule or adding on another molecule. A signal peptide is a short peptide (usually 16-30 amino acids long) present at the N-terminus (or occasionally nonclassically at the C-terminus or internally) of most newly synthesized proteins that are destined toward the secretory pathway. By using a D-amino acid having one or more biochemical functionality, the biochemical properties of the substrate having the D-amino acid derivative protected functional group can be enhanced. In particular, by using signal peptides as one or more functionality of the pathway inside a cell of a substrate, in particular a protein, having the D-amino acid derivative protected functional group can be influenced. In addition, by using pro-peptides as one or more functionality of the substrate, in particular a precursor of a protein, having the D-amino acid derivative protected functional group this substrate can be inactive and can be activated at a predetermined time by an enzymatic activation.

The one or more of a posttranslational modification is not restricted and can e.g. be selected from the group consisting of (2-aminosuccinimyl)acidetic acid (Asp-Gly), (2S)-4-hydroxyleucine, (3R)-3-hydroxyarginine, (3R)-3-hydroxyasparagine, (3R)-3-hydroxyaspartate, (3R)-N4-methyl-3-hydroxy-D-asparagine, (3R,4R)-3,4-dihydroxyproline, (3R,4R)-4,5-dihydroxyisoleucine, (3R,4S)-3,4-dihydroxyproline, (3R,4S)-4-hydroxyisoleucine, (3S)-3-hydroxyasparagine, (3S)-3-hydroxyaspartate, (3S)-3-hydroxyhistine, (3S)-3-hydroxylysine, (3S)-3-methylglutamine, (3S,4R)-3,4-dihydroxyisoleucine, (4R)-4,5-dihydroxyleucine, (4R)-5-hydroxyleucine, (4R)-5-oxoleucine, (4S)-4,5-dihydroxyleucine, (4S)-thiazoline-4-carboxylic acid (Thr-Cys), (5R)-5-hydroxylysine, (5S)-5-hydroxylysine, (E)-2,3-dehydrobutyrine, (E)-2,3-dehydrotyrosine, (Z)-2,3-dehydroaspartate, (Z)-2,3-dehydrobutyrine, (Z)-2,3-dehydrotyrosine, 1-(tryptophan-3-yl)-tryptophan (Trp-Trp) (interchain with W- . . . ), 1-amino-2-propanone, 1-histidyl-3-tyrosine (His-Tyr), 1-thioglycine, 2-(3-methylbutanoyl)-5-hydroxyoxazole-4-carbothionic acid (Leu-Cys), 2-(4-guaninobutanoyl)-5-hydroxyimazole-4-carbothionic acid (Arg-Cys), 2-(cystein-S-ylcarbonyl)-3-methyl-4-(glutam-5-yloxy)methylindole (Glu-Cys), 2-(S-cysteinyl)-histidine (Cys-His), 2-(S-cysteinyl)-methionine (Cys-Met), 2-(S-cysteinyl)pyruvic acid O-phosphothioketal, 2,3-dehydroalanine (Cys), 2,3-dehydroalanine (Ser), 2,3-dehydroalanine (Tyr), 2,3-dehydrobutyrine, 2,3-dehydrotyrosine, 2,4,5-topaquinone, 2-cysteinyl-6-hydroxytryptophan sulfoxid (Trp-Cys), 2-cysteinyl-D-allo-threonine (Cys-Thr), 2-cysteinyl-D-phenylalanine (Cys-Phe), 2-cysteinyl-L-phenylalanine (Cys-Phe), 2-hydroxyproline, 2-iminomethyl-5-imazolinone (Gln-Gly), 2-iminomethyl-5-imazolinone (Glu-Gly), 2-iminomethyl-5-imazolinone (Met-Gly), 2-methylglutamine, 2-methylsulfonyltryptophan, 2-oxo-5,5-dimethylhexanoate, 2-oxobutanoic acid, 2-tetrahydro-2-pyryl-5-imazolinone (Lys-Gly), 3-(04-tyrosyl)-valine (Val-Tyr), 3-(S-cysteinyl)-tyrosine (Cys-Tyr), 3-(S-cysteinyl)-tyrosine (Cys-Tyr), 3-(S-cysteinyl)-tyrosine (Tyr-Cys), 3,3-dimethylmethionine, 3,4,5-trihydroxyphenylalanine, 3,4-dihydroxyarginine, 3,4-dihydroxyphenylalanine, 3,4-dihydroxyproline, 3-bromotyrosine, 3-cysteinyl-aspartic acid (Cys-Asp), 3-farnesyl-2,N2-cyclotryptophan, 3-geranyl-2,N2-cyclotryptophan, 3′-histidyl-3-tyrosine (His-Tyr), 3-hydroxyasparagine, 3-hydroxyaspartate, 3-hydroxy-D-valine, 3-hydroxyphenylalanine, 3-hydroxyproline, 3-hydroxypyrine-2,5-dicarboxylic acid (Ser-Cys) (with S- . . . ), 3-hydroxypyrine-2,5-dicarboxylic acid (Ser-Ser) (with C- . . . ), 3-hydroxytryptophan, 3-hydroxyvaline (Thr), 3-hydroxyvaline (Val), 3-methyl-D-valine, 3-methylisoleucine, 3-methylthioaspartic acid, 3-methylvaline, 3-nitrotyrosine, 3-oxoalanine (Cys), 3-oxoalanine (Ser), 3-phenyllacidtic acid, 3-prenyl-2,N2-cyclotryptophan, 4-(1-hydroxyethyl)-7-isoleucino-2-(threonin-03-ylcarbonyl)-7,8-dihydroquinolin-8-ol (Ile-Thr), 4,5,5-trihydroxyleucine, 4,5-dihydroxylysine, 4-aspartylphosphate, 4-carboxyglutamate, 4-cysteinyl-glutamic acid (Cys-Glu), 4-cysteinyl-tryptophylquinone (Cys-Trp), 4-hydroxyarginine, 4-hydroxyglutamate, 4-hydroxylysine, 4-hydroxyproline, 5-(methoxymethyl)thiazole-4-carboxylic acid (Val-Cys), 5-amino-pipereine-2,5-dicarboxylic acid (Ser-Cys)(with S- . . . ), 5-amino-pipereine-2,5-dicarboxylic acid (Ser-Ser) (with C- . . . ), 5-chlorotryptophan, 5-glutamyl 2-aminoadipic acid, 5-glutamyl dopamine, 5-glutamyl glutamate, 5-glutamyl glycerylphosphorylethanolamine, 5-glutamyl glycine, 5-glutamyl histamine, 5-glutamyl N2-lysine, 5-glutamyl N2-ornithine, 5-glutamyl noradrenaline, 5-glutamyl polyglutamate, 5-glutamyl polyglycine, 5-glutamyl serotonin, 5-hydroxy-3-methylproline (Ile), 5-hydroxylysine, 5-imazolinone (Ala-Gly), 5-imazolinone (Asn-Gly), 5-imazolinone (Cys-Gly), 5-imazolinone (Lys-Gly), 5-imazolinone (Ser-Gly), 5-methylarginine, 5-methyloxazole-4-carboxylic acid (Cys-Thr), 5-methyloxazole-4-carboxylic acid (Ser-Thr), 5-methyloxazole-4-carboxylic acid (Thr-Thr), 5-methyloxazoline-4-carboxylic acid (Ser-Thr), 5-methylthiazole-4-carboxylic acid (Asn-Cys), 5-tyrosyl-5-aminotyrosine (Tyr-Tyr) (interchain with Y- . . . ), 6-(S-cysteinyl)-8alpha-(pros-histyl)-FAD (His-Cys), 6-bromotryptophan, 6-chlorotryptophan, 7-hydroxytryptophan, ADP-alpha-D-ribosylarginine, ADP-ribosyl aspartic acid, ADP-ribosyl glutamic acid, ADP-ribosylarginine, ADP-ribosylasparagine, ADP-ribosylcysteine, ADP-ribosyldiphthamide, ADP-ribosylglycine, ADP-ribosylhistine, ADP-ribosylserine, ADP-ribosyltyrosine, ADP-riboxanated arginine, Alanine amide, Alanine derivative, Alanine isoaspartyl cyclopeptide (Ala-Asn), Allysine, Aminomalonic acid (Ser), Arginine amide, Arginine derivative, Asparagine amide, Aspartate 1-(chondroitin 4-sulfate)-ester, Aspartic acid 1-[(3-aminopropyl)(5-adenosyl)phosphono]amide, Aspartic acid 1-amide, Aspartyl aldehyde, Asymmetric dimethylarginine, Beta-decarboxylated aspartate, Beta-methyllanthionine (Cys-Thr), Beta-methyllanthionine (Thr-Cys), Beta-methyllanthionine sulfoxe (Thr-Cys), Blocked amino end (Ala), Blocked amino end (Arg), Blocked amino end (Asn), Blocked amino end (Asp), Blocked amino end (Asx), Blocked amino end (Cys), Blocked amino end (Gln), Blocked amino end (Glu), Blocked amino end (Gly), Blocked amino end (Ile), Blocked amino end (Leu), Blocked amino end (Met), Blocked amino end (Pro), Blocked amino end (Ser), Blocked amino end (Thr), Blocked amino end (Val), Blocked amino end (Xaa), Blocked carboxyl end (Arg), Blocked carboxyl end (His), Bromohistine, Cholesterol glycine ester, Cis-14-hydroxy-10,13-dioxo-7-heptadecenoic acid aspartate ester, Citrulline, C-linked (Man) hydroxytryptophan, C-linked (Man) tryptophan, Cyclo[(prolylserin)-O-yl]cysteinate, Cyclopeptide (Ala-Arg), Cyclopeptide (Ala-Ile), Cyclopeptide (Ala-Pro), Cyclopeptide (Arg-Cys) (interchain with C- . . . ), Cyclopeptide (Asn-Gly), Cyclopeptide (Asp-Asn), Cyclopeptide (Cys-Arg) (interchain with R- . . . ), Cyclopeptide (Cys-Ile), Cyclopeptide (Cys-Pro), Cyclopeptide (Glu-Asn), Cyclopeptide (Gly-Arg), Cyclopeptide (Gly-Asn), Cyclopeptide (Gly-Asp), Cyclopeptide (Gly-Pro), Cyclopeptide (His-Asn), Cyclopeptide (His-Asp), Cyclopeptide (His-Pro), Cyclopeptide (Ile-Lys), Cyclopeptide (Ile-Pro), Cyclopeptide (Leu-Leu), Cyclopeptide (Leu-Pro), Cyclopeptide (Leu-Trp), Cyclopeptide (Lys-Asp), Cyclopeptide (Met-Pro), Cyclopeptide (Phe-Pro), Cyclopeptide (Pro-Met), Cyclopeptide (Pro-Tyr), Cyclopeptide (Ser-Asn), Cyclopeptide (Ser-Gly), Cyclopeptide (Ser-Lys), Cyclopeptide (Ser-Pro), Cyclopeptide (Trp-Pro), Cyclopeptide (Tyr-Pro), Cyclopeptide (Val-Pro), CysO-cysteine adduct, Cysteine amide, Cysteine derivative, Cysteine methyl disulfe, Cysteine methyl ester, Cysteine persulfe, Cysteine sulfenic acid (—SOH), Cysteine sulfinic acid (—SO2H), Cysteine sulfonic acid (—SO3H), Cysteinyl-selenocysteine (Cys-Sec), Cysteinyl-selenocysteine (Sec-Cys), D-4-hydroxyvaline, D-alanine (Ala), D-alanine (Ser), D-allo-isoleucine, D-asparagine, Deamated asparagine, Deamated glutamine, Decarboxylated threonine, Deoxyhypusine, Diiodotyrosine, Dimethylated arginine, Diphosphoserine, Diphosphothreonine, Diphthamide, Dityrosine (Tyr-Tyr) (interchain with Y— . . . ), D-lacidtate, D-leucine, D-methionine, D-phenylalanine, D-serine (Cys), D-serine (Ser), D-threonine, D-tryptophan, D-valine, FMN phosphoryl serine, FMN phosphoryl threonine, Glutamate methyl ester (Gln), Glutamate methyl ester (Glu), Glutamic acid 1-amide, Glutamine amide, Glutamine derivative, Glycine amide, Glycine radical, Glycyl adenylate, Glycyl cysteine dithioester (Cys-Gly) (interchain with G- . . . ), Glycyl cysteine dithioester (Gly-Cys) (interchain with C- . . . ), Glycyl cysteine thioester (Cys-Gly) (interchain with G- . . . ), Glycyl cysteine thioester (Gly-Cys) (interchain with C- . . . ), Glycyl lysine isopeptide (Gly-Lys) (interchain with K- . . . ), Glycyl lysine isopeptide (Lys-Gly) (interchain with G- . . . ), Glycyl serine ester (Gly-Ser) (interchain with S- . . . ), Glycyl serine ester (Ser-Gly) (interchain with G- . . . ), Glycyl threonine ester (Gly-Thr) (interchain with T- . . . ), Glycyl threonine ester (Thr-Gly) (interchain with G- . . . ), GPI-anchor amated alanine, GPI-anchor amated asparagine, GPI-anchor amated aspartate, GPI-anchor amated carboxyl end, GPI-anchor amated cysteine, GPI-anchor amated glycine, GPI-anchor amated serine, GPI-anchor amated threonine, GPI-like-anchor amated alanine, GPI-like-anchor amated asparagine, GPI-like-anchor amated aspartate, GPI-like-anchor amated glycine, GPI-like-anchor amated serine, Histine amide, Hydroxyarginine, Hydroxyproline, Hypusine, Iodotyrosine, Isoaspartyl cysteine isopeptide (Cys-Asn), Isoaspartyl glycine isopeptide (Asn-Gly), Isoaspartyl glycine isopeptide (Asp-Gly), Isoaspartyl glycine isopeptide (Gly-Asn), Isoaspartyl glycine isopeptide (Gly-Asp), Isoaspartyl lysine isopeptide (Asn-Lys) (interchain with K- . . . ), Isoaspartyl lysine isopeptide (Lys-Asn), Isoaspartyl lysine isopeptide (Lys-Asn) (interchain with N- . . . ), Isoaspartyl lysine isopeptide (Lys-Asp), Isodityrosine (Tyr-Tyr), Isoglutamyl cysteine thioester (Cys-Gln), Isoglutamyl glycine isopeptide (Gly-Glu), Isoglutamyl lysine isopeptide (Gln-Lys) (interchain with K- . . . ), Isoglutamyl lysine isopeptide (Glu-Lys) (interchain with K- . . . ), Isoglutamyl lysine isopeptide (Lys-Gln), Isoglutamyl lysine isopeptide (Lys-Gln) (interchain with Q- . . . ), Isoglutamyl lysine isopeptide (Lys-Glu) (interchain with E- . . . ), Isoleucine amide, Isoleucine derivative, L-allo-isoleucine, Lanthionine (Cys-Ser), Lanthionine (Ser-Cys), Leucine amide, Leucine methyl ester, Lysine amide, Lysine derivative, Lysine methyl ester, Lysine tyrosylquinone (Lys-Tyr), Lysine tyrosylquinone (Tyr-Lys), Lysinoalanine (Ser-Lys), Lysino-D-alanine (Lys), Methionine (R)-sulfoxe, Methionine (S)-sulfoxe, Methionine amide, Methionine derivative, Methionine sulfone, Methionine sulfoxe, Methylhistine, Murein peptoglycan amated serine, N-(12-oxomyristoyl)cysteine, N,N-(cysteine-1,S-diyl)phenylalanine (Cys-Phe), N,N-(cysteine-1,S-diyl)serine (Cys-Ser), N,N,N-trimethylalanine, N,N,N-trimethylglycine, N,N,N-trimethylmethionine, N,N,N-trimethylserine, N,N-dimethylalanine, N,N-dimethylglycine, N,N-dimethylleucine, N,N-dimethylproline, N,N-dimethylserine, N-[(12R)-12-hydroxymyristoyl]cysteine, N2,N2-dimethylarginine, N2-acidetylarginine, N2-succinyltryptophan, N4,N4-dimethylasparagine, N4-methylasparagine, N4-methyl-D-asparagine, N5-[4-(S-L-cysteinyl)-5-methyl-1H-imazol-2-yl]-L-ornithine (Arg-Cys) (interchain with C- . . . ), N5-[4-(S-L-cysteinyl)-5-methyl-1H-imazol-2-yl]-L-ornithine (Cys-Arg) (interchain with R- . . . ), N5-methylarginine, N5-methylglutamine, N6-(2-hydroxyisobutyryl)lysine, N6-(3,6-diaminohexanoyl)-5-hydroxylysine, N6-(ADP-ribosyl)lysine, N6-(beta-hydroxybutyryl)lysine, N6-(pyroxal phosphate)lysine, N6-(retinylene)lysine, N6,N6,N6-trimethyl-5-hydroxylysine, N6,N6,N6-trimethyllysine, N6,N6-dimethyllysine, N6-1-carboxyethyl lysine, N6-acidetyllysine, N6-biotinyllysine, N6-butyryllysine, N6-carbamoyllysine, N6-carboxylysine, N6-crotonyllysine, N6-formyllysine, N6-glutaryllysine, N6-lacidtoyllysine, N6-lipoyllysine, N6-malonyllysine, N6-methylated lysine, N6-methyllysine, N6-murein peptoglycan lysine, N6-myristoyl lysine, N6-palmitoyl lysine, N6-poly(beta-hydroxybutyryl)lysine, N6-poly(methylaminopropyl)lysine, N6-propionyllysine, N6-stearoyl lysine, N6-succinyllysine, N-acidetylalanine, N-acidetylaspartate, N-acidetylcysteine, N-acidetylglutamate, N-acidetylglycine, N-acidetylisoleucine, N-acidetylmethionine, N-acidetylproline, N-acidetylserine, N-acidetylthreonine, N-acidetyltyrosine, N-acidetylvaline, N-alpha-linked (Rha) arginine, N-beta-linked (GlcNacid) arginine, N-carbamoylalanine, N-D-glucuronoyl glycine, N-formylglycine, N-formylmethionine, Nitrated tyrosine, N-linked (DATDGlc) asparagine, N-linked (GalNacid) asparagine, N-linked (GalNacid . . . ) (glycosaminoglycan) asparagine, N-linked (GalNacid . . . ) asparagine, N-linked (Glc) (glycation) arginine, N-linked (Glc) (glycation) histine, N-linked (Glc) (glycation) isoleucine, N-linked (Glc) (glycation) lysine, N-linked (Glc) (glycation) valine, N-linked (Glc) arginine, N-linked (Glc) asparagine, N-linked (Glc . . . ) arginine, N-linked (Glc . . . ) asparagine, N-linked (GlcNacid) asparagine, N-linked (GlcNacid . . . ) (complex) arginine, N-linked (GlcNacid . . . ) (complex) asparagine, N-linked (GlcNacid . . . ) (high mannose) arginine, N-linked (GlcNacid . . . ) (high mannose) asparagine, N-linked (GlcNacid . . . ) (hybr) arginine, N-linked (GlcNacid . . . ) (hybr) asparagine, N-linked (GlcNacid . . . ) (keratan sulfate) arginine, N-linked (GlcNacid . . . ) (keratan sulfate) asparagine, N-linked (GlcNacid . . . ) (paucimannose) arginine, N-linked (GlcNacid . . . ) (paucimannose) asparagine, N-linked (GlcNacid . . . ) (polylacidtosaminoglycan) arginine, N-linked (GlcNacid . . . ) (polylacidtosaminoglycan) asparagine, N-linked (GlcNacid . . . ) arginine, N-linked (GlcNacid . . . ) asparagine, N-linked (Hex) arginine, N-linked (Hex) asparagine, N-linked (Hex) tryptophan, N-linked (Hex . . . ) arginine, N-linked (Hex . . . ) asparagine, N-linked (HexNacid) arginine, N-linked (HexNacid) asparagine, N-linked (HexNacid . . . ) arginine, N-linked (HexNacid . . . ) asparagine, N-linked (Lacid) (glycation) lysine, N-linked (Man) tryptophan, N-methylalanine, N-methylglycine, N-methylisoleucine, N-methylleucine, N-methylmethionine, N-methylphenylalanine, N-methylproline, N-methylserine, N-methyltyrosine, N-myristoyl glycine, N-palmitoyl cysteine, N-palmitoyl glycine, N-pyruvate 2-iminyl-cysteine, N-pyruvate 2-iminyl-valine, O-(2-aminoethylphosphoryl)serine, O-(2-cholinephosphoryl)serine, O-(5′-phospho-DNA)-serine, O-(5′-phospho-DNA)-tyrosine, O-(5′-phospho-RNA)-serine, O-(5′-phospho-RNA)-tyrosine, O-(pantetheine 4′-phosphoryl)serine, O-(phosphoribosyl dephospho-coenzyme A)serine, O-(sn-1-glycerophosphoryl)serine, 03-poly(beta-hydroxybutyryl)serine, 0-8alpha-FAD tyrosine, O-acidetylserine, 0-acidetylthreonine, 0-alpha-linked (GlcNacid) threonine, 0-AMP-serine, O-AMP-threonine, 0-AMP-tyrosine, 0-decanoyl serine, 0-decanoyl threonine, 0-di-AMP-tyrosine, 0-hexanoyl serine, O-linked (Ara) hydroxyproline, O-linked (Ara . . . ) hydroxyproline, O-linked (DADDGlc) serine, O-linked (DATDGlc) serine, O-linked (Fuc) serine, O-linked (Fuc) threonine, O-linked (Fuc . . . ) serine, O-linked (Fuc . . . ) threonine, O-linked (FucNacid) serine, O-linked (FucNacid . . . ) serine, O-linked (Gal) hydroxylysine, O-linked (Gal) hydroxyproline, O-linked (Gal) serine, O-linked (Gal) threonine, O-linked (Gal . . . ) hydroxylysine, O-linked (Gal . . . ) hydroxyproline, O-linked (Gal . . . ) serine, O-linked (Gal . . . ) threonine, O-linked (GalNacid) serine, O-linked (GalNacid) threonine, O-linked (GalNacid) tyrosine, O-linked (GalNacid . . . ) (keratan sulfate) serine, O-linked (GalNacid . . . ) (keratan sulfate) threonine, O-linked (GalNacid . . . ) serine, O-linked (GalNacid . . . ) threonine, O-linked (GalNacid . . . ) tyrosine, O-linked (GATDGlc) serine, O-linked (Glc) hydroxylysine, O-linked (Glc) serine, O-linked (Glc) threonine, O-linked (Glc) tyrosine, O-linked (Glc . . . ) serine, O-linked (Glc . . . ) tyrosine, O-linked (GlcA) serine, O-linked (GlcNacid) hydroxyproline, O-linked (GlcNacid) serine, O-linked (GlcNacid) threonine, O-linked (GlcNacid) tyrosine, O-linked (GlcNacid . . . ) hydroxyproline, O-linked (GlcNacid . . . ) serine, O-linked (GlcNacid . . . ) threonine, O-linked (GlcNacid . . . ) tyrosine, O-linked (GlcNacid1P) serine, O-linked (GlcNacid6P) serine, O-linked (Hex) hydroxylysine, O-linked (Hex) hydroxyproline, O-linked (Hex) serine, O-linked (Hex) threonine, O-linked (Hex) tyrosine, O-linked (Hex . . . ) hydroxylysine, O-linked (Hex . . . ) hydroxyproline, O-linked (Hex . . . ) serine, O-linked (Hex . . . ) threonine, O-linked (Hex . . . ) tyrosine, O-linked (HexNacid) hydroxyproline, O-linked (HexNacid) serine, O-linked (HexNacid) threonine, O-linked (HexNacid) tyrosine, O-linked (HexNacid . . . ) hydroxyproline, O-linked (HexNacid . . . ) serine, O-linked (HexNacid . . . ) threonine, O-linked (HexNacid . . . ) tyrosine, O-linked (Man) serine, O-linked (Man) threonine, O-linked (Man . . . ) (keratan sulfate) serine, O-linked (Man . . . ) (keratan sulfate) threonine, O-linked (Man . . . ) serine, O-linked (Man . . . ) threonine, O-linked (Man1P) serine, O-linked (Man1P . . . ) serine, O-linked (Man6P) threonine, O-linked (Man6P . . . ) threonine, O-linked (Xyl) serine, O-linked (Xyl . . . ) (chondroitin sulfate) serine, O-linked (Xyl . . . ) (dermatan sulfate) serine, O-linked (Xyl . . . ) (glycosaminoglycan) serine, O-linked (Xyl . . . ) (glycosaminoglycan) threonine, O-linked (Xyl . . . ) (heparan sulfate) serine, O-linked (Xyl . . . ) (keratan sulfate) threonine, O-linked (Xyl . . . ) serine, Omega-hydroxyceramide glutamate ester, Omega-N-methylarginine, Omega-N-methylated arginine, O-methylthreonine, O-octanoyl serine, O-octanoyl threonine, O-palmitoleoyl serine, O-palmitoyl serine, O-palmitoyl threonine, O-tri-AMP-tyrosine, O-UMP-histine, O-UMP-serine, O-UMP-threonine, O-UMP-tyrosine, Oxazole-4-carboxylic acid (Cys-Ser), Oxazole-4-carboxylic acid (Gly-Ser), Oxazole-4-carboxylic acid (Ile-Ser), Oxazole-4-carboxylic acid (Ser-Ser), Oxazoline-4-carboxylic acid (Cys-Ser), Pentaglycyl murein peptoglycan amated alanine, Pentaglycyl murein peptoglycan amated threonine, peptide (Met-Gly) (interchain with G- . . . ), Phenylalanine amide, Phosphatylethanolamine amated glycine, Phosphatylserine amated glycine, Phosphoarginine, Phosphocysteine, Phosphohistine, Phosphoserine, Phosphothreonine, Phosphotyrosine, PolyADP-ribosyl aspartic acid, PolyADP-ribosyl glutamic acid, Proline 5-hydroxy-oxazole-4-carbothionic acid (Pro-Cys), Proline amide, Pros-8alpha-FAD histine, Pros-methylhistine, Pros-phosphohistine, Pyrine-2,5-dicarboxylic acid (Ser-Cys) (with S- . . . ), Pyrine-2,5-dicarboxylic acid (Ser-Ser) (with C- . . . ), Pyrrolone carboxylic acid, Pyrrolone carboxylic acid (Glu), Pyrroloquinoline quinone (Glu-Tyr), Pyruvic acid (Cys), Pyruvic acid (Ser), Pyruvic acid (Tyr), S-(15-deoxy-Delta12,14-prostaglandin J2-9-yl)cysteine, S-(2,3-dicarboxypropyl)cysteine, S-(2-aminovinyl)-3-methyl-D-cysteine (Thr-Cys), S-(2-aminovinyl)-D-cysteine (Cys-Cys), S-(2-aminovinyl)-D-cysteine (Ser-Cys), S-(2-aminovinyl)-L-cysteine (Cys-Cys), S-(2-succinyl)cysteine, S-(4-hydroxycinnamyl)cysteine, S-(coelenterazin-3a-yl)cysteine, S-(dipyrrolylmethanemethyl)cysteine, S-12-hydroxyfarnesyl cysteine, S-4a-FMN cysteine, S-6-FMN cysteine, S-8alpha-FAD cysteine, S-archaeol cysteine, S-bacidillithiol cysteine disulfe, S-carbamoylcysteine, S-cGMP-cysteine, S-cyanocysteine, S-cysteinyl 3-(oxosulfanyl)alanine (Cys-Cys), S-cysteinyl cysteine, S-diacidylglycerol cysteine, Serine amide, Serine microcin E492 serophore ester, S-farnesyl cysteine, S-geranylgeranyl cysteine, S-glutathionyl cysteine, S-linked (Gal) cysteine, S-linked (Gal . . . ) cysteine, S-linked (Glc) cysteine, S-linked (Glc . . . ) cysteine, S-linked (GlcNacid) cysteine, S-linked (GlcNacid . . . ) cysteine, S-linked (Hex) cysteine, S-linked (Hex . . . ) cysteine, S-linked (HexNacid) cysteine, S-linked (HexNacid . . . ) cysteine, S-Lysyl-methionine sulfilimine (Lys-Met) (interchain with M- . . . ), S-Lysyl-methionine sulfilimine (Met-Lys) (interchain with K- . . . ), S-methylcysteine, S-methylmethionine, S-nitrosocysteine, S-palmitoleoyl cysteine, S-palmitoyl cysteine, S-poly(beta-hydroxybutyryl)cysteine, S-selanylcysteine, S-stearoyl cysteine, Sulfoserine, Sulfothreonine, Sulfotyrosine, Symmetric dimethylarginine, Tele-(1,2,3-trihydroxypropan-2-yl)histine, Tele-8alpha-FAD histine, Tele-8alpha-FMN histine, Tele-methylhistine, Tele-phosphohistine, Thiazole-4-carboxylic acid (Arg-Cys), Thiazole-4-carboxylic acid (Asn-Cys), Thiazole-4-carboxylic acid (Cys-Cys), Thiazole-4-carboxylic acid (Glu-Cys), Thiazole-4-carboxylic acid (Gly-Cys), Thiazole-4-carboxylic acid (Ile-Cys), Thiazole-4-carboxylic acid (Phe-Cys), Thiazole-4-carboxylic acid (Pro-Cys), Thiazole-4-carboxylic acid (Ser-Cys), Thiazole-4-carboxylic acid (Thr-Cys), Thiazole-4-carboxylic acid (Val-Cys), Thiazoline linkage to a ring-opened DNA abasic site, Thiazoline-4-carboxylic acid (Phe-Cys), Threonine 5-hydroxy-oxazole-4-carbonthionic acid (Thr-Cys), Threonine amide, Threonine methyl ester, Threonyl lysine isopeptide (Lys-Thr) (interchain with T- . . . ), Threonyl lysine isopeptide (Thr-Lys) (interchain with K- . . . ), Thyroxine, Triiodothyronine, Trithiocysteine (Cys-Cys), Tryptophan amide, Tryptophan derivative, Tryptophan tryptophylquinone (Trp-Trp), Tryptophylquinone, Tryptophyl-tyrosyl-methioninium (Trp-Tyr) (with M- . . . ), Tryptophyl-tyrosyl-methioninium (Tyr-Met) (with W- . . . ), Tyrosine amide, Valine amide.

A posttranslational modification (PTM) is the covalent and commonly enzymatic modification of proteins following protein biosynthesis.

According to certain embodiments, the one or more artificial derivatisations can be selected from the group consisting of a polymer, a chemical chelator, a polyaminocarboxylate, deferroxamine, deferasirox, deferipone, and the like.

The polymer is not particularly restricted and e.g. can be selected from the group consisting of polyethylen glycols, dextrane, and the like.

According to certain embodiments, the chemical chelator, which is not particularly restricted, can include a cyclic polyamine. The cyclic polyamine can e.g. be selected from the group consisting of cyclen, cyclam, and the like.

According to certain embodiments, the polyaminocarboxylate can be selected from the group consisting of (2,2′,2″-(1,4,7-triazacyclononane-1,4,7-triyl)-triacetic acid (NOTA), 2,2′,2″,2″′-(1,4,7,10-tetraazacyclododecane-1,4,7,10-tetrayl)-tetraacetic acid (DOTA), 1,4,8,11-Tetraazacyclotetradecane-1,4,8,11-tetraacetic acid (TETA), and the like.

By using one of these D-amino acid derivatives, the properties of the substrate having the D-amino acid derivative protected functional group can be adapted as described-above in detail. By using a combination of one or more of the one or more functionality, posttranslational modification and artificial derivatisation, the properties of the substrate having the D-amino acid derivative protected functional group can be adapted effectively, selectively and precisely.

In the method, the at least one substrate is not particularly restricted, as long as it has at least one D-amino acid and/or D-amino acid derivative protected functional group (the functional group not being restricted), and can be one substrate or more than one substrates, e.g. two or more, three or more, etc., that can be mixed. According to certain embodiments, one substrate is used in the method. Generally, a suitable substrate can be any organic compound, e.g. used for synthesizing low-molecular active ingredients.

According to certain embodiments, the substrate can be selected from the group consisting of a carbohydrate, a carbohydrate derivative, a nucleotide, an oligonucleotide, a polynucleotide, a nucleotide derivative, an oligonucleotide derivative, a polynucleotide derivative, an amino acid; a peptide; an oligopeptide; a polypeptide; a protein; a peptide intermediate; a peptide derivative; an oligopeptide derivative; a polypeptide derivative; and mixtures thereof; particularly wherein the substrate has at least two functional groups. According to certain embodiments, the substrate can be selected from the group consisting of a carbohydrate, a carbohydrate derivative, and mixtures thereof; particularly wherein the substrate has at least two functional groups. In a specific embodiment, the substrate can be selected from the group consisting of an amino acid; a peptide; an oligopeptide; a polypeptide; a protein; a peptide intermediate; a peptide derivative; an oligopeptide derivative, a polypeptide derivative; and mixtures thereof; particularly wherein the substrate has at least two functional groups. According to certain embodiments, the amino acid, the peptide, the oligopeptide, the polypeptide, and the peptide intermediate can be an L-amino acid, an L-peptide, an L-oligopeptide, an L-polypeptide, and an L-peptide intermediate.

According to certain embodiments, the amino acid, the peptide, the oligopeptide, the polypeptide, the protein, and the peptide intermediate can be an enzyme precursor.

In the invention, the carbohydrate is not particularly limited. The carbohydrates can include monosaccharides, disaccharides, oligosaccharides, and polysaccharides. The monosaccharide is not particularly limited and can e.g. be selected from the group consisting of a diose, a triose, a tetrose, a pentose, and a hexose. The diose therein can include glycolaldehyde, the triose can e.g. be selected from the group consisting of glyceraldehyde, and dihydroxyacetone, the tetrose can e.g. be selected from the group consisting of erythrose, threose and erythrulose, the pentose can e.g. be selected from the group consisting of ribose, arabinose, xylose, lyxose, deoxyribose, ribulose, and xylulose, and the hexose can be selected from the group consisting of allose, altrose, glucose, mannose, gulose, idose, galactose, talose, and fructose. A disaccharide is a saccharide formed when two monosaccharides are linked by a glycosidic bond. For example, the disaccharide can be selected from the group consisting of cellobiose, gentiobiose, sucrose, lactose, trehalose, and maltose. An oligosaccharide is a saccharide formed when a small number, typically three to ten, of monosaccharides are linked by glycosidic bonds. The oligosaccharides can have linear or branched structures. For example, the oligosaccharide can be selected from the group consisting of fructooligosaccharide, raffinose, and galactooligosaccharide. A polysaccharide is a saccharide containing a large number, at least 11, of monosaccharides that are linked by glycosidic bonds. The polysaccharides can have linear or branched structures. For example, the polysaccharide can be selected from the group consisting of glycogen, starch such as amylose and amylopectin, pectins, chitin, callose, and cellulose.

The carbohydrate derivative can be based on a carbohydrate, as above, except that one or more chemical group can be introduced to the carbohydrate and/or that one or more functional group of the carbohydrate can be substituted by at least one chemical group and/or that the carbohydrate is in the form of a salt. According to certain embodiments, one or more hydroxyl group of the carbohydrate can be substituted by at least one other chemical group that is not particularly restricted, and if more than one chemical group is present, these can be the same or different. The one or more chemical group can be e.g. selected from the group consisting of a hydrogen, a halogen, an alkyl group, an alkenyl group, e.g. a vinyl group, an allyl group, etc., an alkinyl group, an aromatic group, an ester group, a carboxylic group, an acetyl group, an ether group, a keto group, an aldehyde group, an amine group, an amide group, an imine group, an imide group, an acetylamino group, an acetal group, a hemiacetal group, a nitrile group, a thiol group, a sulfide group, and a posphine group.

According to certain embodiments, the one or chemical group can be selected from the group consisting of a hydrogen, a halogen, an alkyl group, an alkenyl group, an alkinyl group, an ester group, a carboxylic group, an acetyl group, an ether group, a keto group, an aldehyde group, an amine group, and an acetylamino group. According to certain embodiments, the alkyl group can have 1 to 20 carbon atoms. According to certain embodiments, the alkenyl group can have 1 to 20 carbon atoms. According to certain embodiments, the alkinyl group can have 1 to 20 carbon atoms.

In the invention, the nucleotide, oligonucleotide and polynucleotide are not particularly restricted. A nucleotide derivative includes one or more modifications. The nucleotide derivative can be in form of a salt. The nucleotide derivative can include a modified nucleobase. The modified nucleobase can be based on the nucleobase described above except that one or more chemical group can be introduced to the nucleobase described above and/or that one or more functional group of the nucleobase described above can be substituted by at least one chemical group, e.g. selected from the group consisting of a hydrogen, a halogen, an alkyl group, an alkenyl group, an alkinyl group, an aromatic group, amine group, an amide group, an imine group, an imide group, an acetylamino group, a thiol group. The same derivatisations can apply to oligonucleotides and polynucleotides

The amino acid that can be used as a substrate is not particularly limited and is preferably an L-amino acid. According to certain embodiments, the amino acid can be selected from the group consisting of a proteinogenic amino acid and a non-proteinogenic amino acid. The proteinogenic amino acid can be selected from the group consisting of alanine (ala, A), arginine (arg, R), asparagine (asn, N), aspartate (asp, D), cysteine (cys, C), glutamine (gln, Q), glutamate (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), valine (val, V), selenocysteine (sec, U), and pyrrolysine (pyl, O). The non-proteinogenic amino acid is not restricted and can e.g. be selected from the group consisting of carnitine, GABA (γ-aminobutyric acid), levothyroxine, hydroxyproline, 2-aminoisobutyric acid, gamma-aminobutyiric acid, ornithine, citrulline and selenomethionine.

A peptide within the invention can be, according to certain embodiments an L-peptide. The amino acids forming the peptide are not particularly limited. According to certain embodiments, the amino acid forming the peptide are selected from the group consisting of alanine (ala), lysine (lys), and tyrosine (tyr). An oligopeptide can likewise be an L-oligopeptide. The amino acids forming the oligopeptide are not particularly limited. A polypeptide can be an L-polypeptide.

The amino acids forming the polypeptide are not particularly limited. Likewise, the amino acids forming the protein are not particularly limited.

A peptide intermediate is a compound that is present during the synthesis of a peptide, an oligopeptide, a polypeptide, a protein or an enzyme. According to certain embodiments, the peptide intermediate can include a peptide that can be modified by one or more chemical groups, in particular a protecting group. According to certain embodiments, functional groups of the amino acids can be protected by a protecting group to prevent an undesired reaction of these functional groups. According to certain embodiments, the N-terminus of the peptide can be modified by one or more chemical group in particular a protecting group. The protecting group is not particularly limited. According to certain embodiments, the protecting group can be selected from the group consisting of a benzoyl group (Bz), a fluorenylmethyloxycarbonyl group (Fmoc), a tert-butyloxycarbonyl group (Boc), an acetale group (Ac), a succinic group (Suc), benzyloxycarbonyl group (Z), a 2-azidomethylbenzyl group (Ambz), a 2-aminobenzoic acid group (Abz), a biotin group, and mixtures thereof. The peptide intermediate can further include a linker, which is e.g. the linkage between the peptide intermediate and a resin. The linker is not particularly limited and can e.g. be selected from the group consisting of a Wang linker, a Rink linker, Merrifield linker, a 4-methylbenzhydrylamine linker (MBHA), a phenylacetamidomethyl (PAM) linker, a nitrobenzyl linker, and an allyl linker. The Wang linker can include p-alkoxybenzylester linker. The Rink linker can include a 4-(2′,4′-dimethoxyphenyl-4-hydroxymethyl-phenoxy) linker, and a 4-(2′,4′-Dimethoxyphenyl-4-methylamide-phenoxy) linker. The Merrifield linker can include a benzyl linker. The resin is not particularly limited and can e.g. be selected from the group consisting of polystyrenes.

In a peptide derivative and/or an oligopeptide derivative and/or polypeptide derivative, one or more chemical groups can be introduced to the peptide and/or one or more functional groups of the peptide can be substituted by at least one chemical group, as e.g. given above for the carbohydrate derivative. In addition, the peptide derivative can be a peptide in form of a salt.

According to certain embodiments, the method can further comprises adding a buffer to the reaction mixture, wherein the pH value of the reaction mixture can be in the range from 3 to 13. The buffer can be provided in addition to at least one substrate having at least one D-amino acid and/or D-amino acid derivative-protected functional group, the D-stereospecific hydrolytic enzyme, and at least one solvent. It can be provided and mixed with the at least one substrate having at least one D-amino acid and/or D-amino acid derivative-protected functional group, the D-stereospecific hydrolytic enzyme and the at least one solvent.

The buffer is not particularly restricted and can e.g. be selected from the group consisting of acetate buffer, 2-(N-morpholino)ethanesulfonic acid (MES) buffer, sodium phosphate (NaPi) buffer, potassium phosphate (KPi) buffer, 2-(4-(2-hydroxyethyl) piperazin-1-yl)-ethane-1-sulfonic acid (HEPES) buffer, tris-(hydroxymethyl)-aminomethane (Tris) buffer, piperazine-N,N′-bis(2-ethanesulfonic acid) (PIPES) buffer, bis-(2-hydroxyethyl)-imino-tris-(hydroxymethyl)-methane (Bis-Tris) buffer, 1,3-bis-(tris-(hydroxymethyl)-methylamino)-propane (Bis-Tris propane) buffer, N-(2-acetamido)iminodiacetic acid (ADA) buffer, N-(2-acetamido)-2-aminoethanesulfonic acid (ACES) buffer, acetamido glycine buffer, 2-hydroxy-3-morpholinopropanesulfonic acid (MOPSO) buffer, N,N-bis-(2-hydroxyethyl)-2-amino-ethansulfonic acid (BES) buffer, 3-(N-morpholino)-propanesulfonic acid (MOPS) buffer, 2-((1,3-dihydroxy-2-(hydroxymethyl)-propan-2-yl)-amino)ethane-1-sulfonic acid (TES) buffer, cholamine chloride hydrochloride, 4-(N-morpholino)-butanesulfonic acid (MOBS) buffer, 3-N-bis-(hydroxyethyl)-amino-2-hydroxy-propansulfonic acid (DIPSO) buffer, 3-((1,3-dihydroxy-2-(hydroxymethyl)-propan-2-yl)-amino)-2-hydroxypropane-1-sulfonic acid (TAPSO) buffer, triethanolamine buffer, 4-(2-hydroxyethyl)-piperazin-1-(2-hydroxy-propansulfonic acid) (HEPPSO) buffer, piperazin-N,N′-bis-(2-hydroxypropansulfonic acid) (POPSO) buffer, N-(tri(hydroxymethyl)-methyl)-glycine (Tricine) buffer, glycylglycine buffer, gylcinamide buffer, (bis(2-hydroxyethyl)-amino)acetic acid (BICINE) buffer, N-tris(hydroxymethyl)-methyl-3-aminopropanesulfonic acid (TAPS) buffer, 2-amino-2-methyl-1,3-propanediol (AMPD) buffer, N-tris(hydroxymethyl)-methyl-4-aminobutanesulfonic acid (TABS) buffer, N-(1,1-dimethyl-2-hydroxyethyl)-3-amino-2-hydroxypropansulfonic acid (AMPSO) buffer, N-cyclohexyl-2-aminoethanesulfonic acid (CHES) buffer, 3-(cyclohexylamino)-2-hydroxy-1-propansulfonic acid (CAPSO) buffer, (2-Amino-2-methylpropan-1-ol) AMP buffer and 3-(cyclohexylamino)propane-1-sulfonic acid (CAPS) buffer. According to certain embodiments, the buffer is selected from the group consisting of acetate buffer, 2-(N-morpholino)ethanesulfonic acid (MES) buffer, sodium phosphate (NaPi), N-tris(hydroxymethyl)-methyl-3-aminopropanesulfonic acid (TAPS) buffer, and 3-(cyclohexylamino)propane-1-sulfonic acid (CAPS) buffer. By using these buffers, the pH value can be kept constant. A constant pH value can improve the effectivity of the hydrolyzing step and the activity of the D-stereospecific hydrolytic enzyme in the hydrolyzing step can be enhanced. According to certain embodiments, the pH value of the reaction mixture can be in the range from 3 to 13, preferably in the range from 5 to 11, more preferably from 7 to 10, more preferably from 7 to 9. By carrying out the deprotecting at such pH value within the above range, the effectivity of the hydrolyzing step can be improved and the activity of the D-stereospecific hydrolytic enzyme in the hydrolyzing step can be enhanced. The concentration of the buffer in the reaction mixture is not limited and can be e.g. in the range from 1 mmol to 1 mol, more preferably from 10 mmol to 500 mmol, more preferably from 15 mmol to 300 mmol, more preferably from 20 mmol to 200 mmol, based on the reaction mixture. By carrying out the deprotecting with the buffer concentration within the above range, the pH value can be controlled precisely. In addition, the effectivity of the hydrolyzing step can be improved and the activity of the D-stereospecific hydrolytic enzyme in the hydrolyzing step can be enhanced.

According to certain embodiments, the functional group of the substrate that is protected can be selected from the group consisting of an amino group, a carboxyl group, a hydroxyl group, a sulfhydryl group, and a selenohydril group, e.g. an amino group, a hydroxyl group, a sulfhydryl group and/or a selenohydril group, and particularly the D-amino acid and/or D-amino acid derivative can bind via the carbonyl group to the substrate. During the hydrolyzing step, a water molecule e.g. provided from the solvent can react with the above-mentioned at least one functional group of the substrate that is protected, forming a D-amino acid and/or a D-amino acid derivative and an at least partially deprotected substrate with at least one deprotected functional group deprotected from the D-amino acid and/or D-amino acid derivative.

The concentration of the substrate in the reaction mixture is not particularly limited and can e.g. be in the range from 100 nM to 1 M, preferably from 1 μM to 100 mM, more preferably 5 μM to 50 mM, more preferably 10 μm to 10 mM, even more preferably 100 μM to 1 mM, based on the reaction mixture. By carrying out the deprotecting with the concentration of the substrate within the above range, the hydrolysis reaction proceeds more effectively.

The concentration of the D-stereospecific hydrolytic enzyme in the reaction mixture is not limited and can be e.g in the range from 1 nM to 200 μM, preferably 100 nM to 100 μM, more preferably 500 nm to 50 μM, more preferably 750 nm to 10 μM, based on the reaction mixture. By carrying out the deprotecting with the concentration of the D-stereospecific hydrolytic enzyme within the above range, the hydrolysis reaction proceeds more effectively.

The at least one solvent is not particularly limited but preferably includes at least water. The solvent can be a mixture of at least two solvents. According to certain embodiments, the at least one solvent can be selected from the group consisting of water, an alcohol, e.g. methanol, ethanol, propanol, isopropanol, butanol, isobutanol and mixtures thereof, an aldehyde, e.g. acetaldehyde, propionaldehyde, butyraldehyde, and mixtures thereof, a ketone, e.g., acetone, butanone, 2-pentanone, 3-pentanone, 3-methylbutanone, and mixtures thereof, an ester, e.g. methyl acetate, ethyl acetate, and mixtures thereof, an amide, e.g. DMF (dimethylformamide), formamide, and mixtures thereof, a nitrile, e.g. acetonitrile, propionitrile, isobutyronitrile, and mixtures thereof, a sulfoxide, e.g. DMSO (dimethylsulfoxide), and mixtures thereof, e.g. selected from the group consisting of water, an alcohol, a nitrile, a sulfoxide, and mixtures thereof, e.g. selected from the group consisting of water, DMSO (dimethylsulfoxide), DMF (dimethylformamide), acetonitrile, methanol and mixtures thereof. According to certain embodiments, the at least one solvent can be water. Using these solvents, the effectivity of the hydrolyzing step and the activity of the D-stereospecific hydrolytic enzyme in the hydrolyzing step can be enhanced.

The concentration of the at least solvent can be in the range from 0.1 to 90%, preferably 0.5 to 70%, more preferably from 1 to 50%, and even preferably from 2% to 35%, even more preferably from 2.5% to 10% based on the volume of the entire solvent.

According to certain embodiments, the step of hydrolyzing can be carried out at a reaction temperature in the range from 0° C. to 50° C., preferably from 5° C. to 40° C., more preferably from 15° C. to 35° C., more preferably from 22° C. to 32° C., even more preferably about 30° C. By carrying out the deprotecting at a temperature within the above range, the hydrolysis reaction proceeds more effectively. In addition, side reactions can be prevented.

The method of the first aspect can further comprise adding at least one additive to the reaction mixture. The additive can be provided in addition to at least one substrate having at least one D-amino acid and/or D-amino acid derivative-protected functional group, the D-stereospecific hydrolytic enzyme, and at least one solvent. The additive is not particularly limited as long as it is compatible with the compounds present in the reaction mixture. In particular, the enzymes applied are compatible with the additive. According to certain embodiments, the at least one additive can be selected from the group consisting of a salt, and an organic compound. The salt can be added to enhance the solubility of the substrate and can be selected from the group consisting of NaCl, KCl, LiCl, CaCl₂, MgCl₂, NaBr, AgCl, MnCl₂, MgSO₄, MnSO₄, ZnSO₄, (NH₄)₂SO₄, and mixtures thereof. NaCl can be preferable according to certain embodiments.

Likewise, the organic compound is not limited and can e.g. be selected from the group consisting of an alkane, an alkene, an alkyne, alcohol, an aldehyde, a ketone, an ester, an ether, an amine, an amide, an imide, a nitrile, a thiol, salts thereof, and mixtures thereof, e.g. from the group consisting of an alcohol, an amine, an amide, a thiol, salts thereof, and mixtures thereof. According to certain embodiments, the organic compound can be selected from the group consisting of DTT (dithiothreitol), DTT derivatives, 2-mercaptoethanol, 2-mercaptoethanol derivatives, urea, urea derivatives, guanidinium chloride, guanidinium chloride derivatives, and mixtures thereof. According to certain embodiments, the additive can be selected from the group consisting of NaCl, KCl, LiCl, CaCl₂, MgCl₂, and mixtures thereof; DTT, DTT derivatives, 2-mercaptoethanol, 2-mercaptoethanol derivatives, urea, urea derivatives, guanidinium chloride, guanidinium chloride derivatives, and mixtures thereof, e.g. from the group consisting of NaCl, DTT, 2-mercaptoethanol, urea, guanidinium chloride, and mixtures thereof. By adding at least one additive, the solubility of one or more of the compounds in the reaction mixture can be enhanced. The concentration of the at least one organic compound is not limited and can be in the range from 1 mM to 5 M, e.g. from 1 mM to 2.5 M, more preferably from 1 mM to 1 M, more preferably from 1 mM to 0.5 M, more preferably from 1 mM to 0.25 M, and even more preferably from 1 mM to 0.1 M. According to certain embodiments, the concentration of a salt, e.g. NaCl, can be in the range from 1 mM to 5 M, more preferably from 1 mM to 2.5 M. By carrying out the deprotecting with the concentration of the additive within the above range, the solubility of one or more of the compounds in the reaction mixture can be further enhanced.

In the method of the first aspect, the D-stereospecific hydrolytic enzyme can, in certain embodiments, be characterized by the following features:
at least one first 3₁₀-helix-2 structure comprising an amino acid sequence SXXK; at least one first α-helix structure comprising an amino acids sequence YSN; and at least one first β-sheet structure comprising an amino acid sequence HXG; wherein X is an amino acid selected from the group consisting of proteinogenic amino acids; wherein the first 3₁₀-helix-2 structure, the first α-helix structure, and the first β-sheet structure are arranged in close proximity.

According to certain embodiments, the proteinogenic amino acids can be selected from the group selected of alanine (ala, A), arginine (arg, R), asparagine (asn, N), aspartate (asp, D), cysteine (cys, C), glutamine (gln, Q), glutamate (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y) and valine (val, V).

According to certain embodiments, the 3₁₀-helix-2 structure is an N-terminal extension of an α-helix-2.

According to certain embodiments, the at least one first 3₁₀-helix-2 structure can comprise an amino acid sequence SXXK at the end of the 3₁₀-helix-2 structure.

According to certain embodiments, the amino acids S and K of the amino acid sequence SXXK in the at least one first 3₁₀-helix-2 structure can be directly involved in the hydrolytic cleavage of at least one D-amino acid and/or D-amino acid derivative-protected functional group of a substrate to obtain a D-amino acid and/or a D-amino acid derivative and a D-amino acid and/or D-amino acid derivative at least partially deprotected substrate. According to certain embodiments, S of the amino acid sequence SXXK can be involved in binding the substrate having at least one D-amino acid and/or D-amino acid derivative-protected functional group during the hydrolysis. According to certain embodiments, S of the amino acid sequence SXXK that can be involved in binding the substrate having at least one D-amino acid and/or D-amino acid derivative-protected functional group during the hydrolysis can be an activated S. In the structure, the serine has an —OH group that can act as a nucleophile, attacking a substrate's carbonyl carbon of the scissile peptide bond. In addition, a pair of electrons on the lysine nitrogen can accept the hydrogen from the serine —OH group, thus coordinating the attack of the peptide bond. According to certain embodiments, X in the acid sequence SXXK can be selected from the group consisting of I, K, L, V, and combinations thereof.

According to certain embodiments, the YSN motif of the first α-helix structure can be located at the N-terminal end of an α-helix-6. In this structure, the tyrosine has an —OH group that can activate a water molecule, supposedly attacking a Ser-substrate ester intermediate bond. The asparagine can support this activation via an electrostatic network generated by E, D, or Q.

According to certain embodiments, the HXG motif of the first β-sheet structure can be involved in the formation of an oxyanion hole. An oxyanion hole is a pocket in the active site of an enzyme that stabilizes transition state negative charge on a deprotonated oxygen. The pocket can consist of backbone amides or positively charged residues. According to certain embodiments, X in the sequence HXG can be selected from the group consisting of G, N, R, and S. According to certain embodiments, the HXG motif can be involved in stabilizing the substrate having at least one D-amino acid and/or D-amino acid derivative protected functional group. The histidine therein is a central part of an electrostatic network allowing the coordination and the sequence of the reaction. The glycin can be involved in forming the oxyanionic hole, which allows the nucleophilic attack of the serine above.

According to certain embodiments, the features of the D-stereospecific hydrolytic enzyme mentioned-above can provide a binding pocket for a substrate having at least one D-amino acid and/or D-amino acid derivative protected functional group.

According to certain embodiments, further at least one second 3₁₀-helix structure comprising an amino acid selected from E, D and Q can be involved in the region for the substrate recognition.

Such structure can enable an interaction of the D-stereospecific hydrolytic enzyme with the substrate. Alternatively or in addition, it can enable an interaction of the D-stereospecific hydrolytic enzyme with a correct coordination of the substrate in the binding pocket.

According to certain embodiments, the first 3₁₀-helix-2 structure, the first α-helix structure, and the first β-sheet structure are arranged in close proximity so that all fragments are close enough to interact with the substrate having at least one D-amino acid and/or D-amino acid derivative protected functional group.

According to certain embodiments, the D-stereospecific hydrolytic enzyme can have an amino acid sequence chosen from SEQ ID No. 1, No. 2, No. 3, No. 4, No. 5, No. 6, No. 7, No. 8 and No. 9. According to certain embodiments, the D-stereospecific hydrolytic enzyme can have an amino acid sequence chosen from SEQ ID No. 1, No. 2 No. 3, No. 4, No. 5, No. 7, No. 8 and No. 9.

SEQ ID No. 1:
ASLQLSQTKELLTIRIQTDAVTKYGKEDAAIDAYLKGEGFSGMALVAKDG
GVLLRKGYGFAGTNKLNRPDSKTRIASITKSFTAASIMQLVEQGKLSLTD
PVSKFVTGIPRGDDITIHMLLSHTSGLQSEFTRSGDVTIEQTIEELRTKQ
LKYEPGTTYLYSNNGYVLLAYVLEQLSGISYSDYVSEHFLTPLGMKNSGT
ATPATPTIQGYILQKNNEWAAAPYYVSQSGTGTLYSTVDDLLKWDTALRA
GKVVSEQSLEAMYTPHSDKNYGYGWIALGLNGEKGVFHNGSGSGYATGML
RNLDSGMTVILLGNHAGMDMTKLLQQVHKLAAEQ;
SEQ ID No. 2:
TIRIQTDAVTKYGKEDAAIDAYLKGEGFSGMALVAKDGEVLLRKGYGFSG
TNKLNHPDAKSRIASLTKSFTAASIMQLVEQGKLSLTDPVSKFVTGIPRG
DDITIHMLLSHTSGLPSEFTRSGNVTIEQTIAELRTKQLKYEPGTTYLYS
NNGYVLLAYVLEQLSGESYADYVSEHFLTPLGMKNSGTATPATATIQGYI
LQKNNEWAAAPYYVSQSGTGTLYSTVDDLLKWDQALRAGKVVSKQSLEAM
YTPHSDKNYGYGWIAINLNGEKGVFHNGSGSGYATGMLRNLDSGMTVILL
GNHAGMDMTKLLQQVHKLAAEQ;
SEQ ID No. 3:
ASLQLSQTKEFLTIRIQTDAVTKYGKEDAAIDAYLKGEGESGMALVAKDG
EVLLRKGYGFSGTNKLNRPDAKSRIASITKSFTAASIMQLVEQGKLSLTD
PVSKFVTGIPRGDDITIHMLLSHTSGLPSEFTRSGDVTIEQTIAELRTKQ
LKYEPGTTYLYSNNGYVLLAYVLEQLAGESYADYVNEHELTPLGMKNSGT
ATPESPTIQGYILQKNNEWAAAPYYVSQSGTGTLYSTVDDLLKWDQALRA
GKVVSEQSLEAMYTPHSDKNYGYGWIAINLNGEKGVFHNGSGSGYATGML
RNLDSGMTVILLGNHAGMDMTKLLQQVHKLAAEQ;
SEQ ID No. 4:
SSVQTSTORDRNSVKQAVRDTLQLGFPGILAKTSEGGKTWSYAAGVANLS
SKKPMKTDFRFRIGSVTKTFTATVVLQLAEENRLNLDDSIEKWLPGVIQG
NGYDDKQITIRQLLNHTSGIAEYTRSKSFDLMDTKKSYRAEELVKMGISM
PPDFAPGKSWSYSNTGYVLLGILIETVTGNSYAEEIENRIIEPLELSNTF
LPGNSSVIPGTKHARGYIQLDGASEPKDVTYYNPSMGSSAGDMISTADDL
NKFFSYLLGGKLLKEQQLKQMLTTVPTGEAALGRYGLGIYETKLPNGVSI
WGHGGSIPGFVTFAGGTLGGKHTLAVNLNSLNAESPDPFKNILLAEFSK;
SEQ ID No. 5:
SSLQTSTQSDRTSVKKAMRDELQLGYPGILAKISKGGKTWSYTAGVADLK
TKKPMKADFRFRIGSVTKTFIATVLLQLSGENRLNLDDSIEKWLPGVIQG
NGYDGNQITIRQILNHTSGIADYVNSKDFDIMDTKKSYTAEEFVKMGISL
PPDFAPGKGWSYSNTGYVLLGILIEKVTGNSYAEEVENRIIEPLDLSNTF
LPGNSSVIPGTKHARGYLQINGASELKDVTYINPGSSDGDMISTADDLNK
FFSCLLGGKLLKEQQLKQMLTTVPTNREGTGYGLGILEIKLPNGVSVWGH
RGAVPGFSTFAGGTLGGKHTLAINSNSLNLNNPEVFKNILLAEFRK;
SEQ ID No. 6:
SSLQTSTQSDRTSVKKAIRDELQLGYPGILAQISKGGKTWSYTAGIADLR
TKKPMKADFRFRIGSVTKTFIATVLLQLSGENRLNLDDSIEKWLPGVIQG
NGYDGNQITIRQILNHTSGIADYINSKDFDIMDTKKSYTAEEFVKMGISL
PPDFAPGKGWSYSNTGYVLLGILIEKVTGNSYAEEVENRIIEPLDLSNTF
LPGNSSVIPGTKHARGYLQLDGASELKDVTYINPGSSDGDMISTADDLNK
FFSYLLGGKLLKEQQLKQMLTTVPTNREGTGYGLGILEIKLPNGVSVWGH
RGGVLGFSTFAGGTLGGKHTLAINSNSFNINNPESFKNVLIAEFSK;
SEQ ID No. 7:
NSLQTSTQRDRNSVKEAMRDTLKLGYPGILAKTSEGGKTWSYAAGVADLS
NKKAMKTDFRFRIGSVTKTFTATVVLQLAGENRLNLDDSIEKWLPGVIQG
NGYDDKQITIRQLLNHTSGIANYTRSKDFNMMDTKKSYTAEGLVKMGISM
PPDFAPGKSWSYSNTGYVLLGILIEKVTGNSYAEEIENRIIEPLELANTF
LPGNSSVIPGTKHARGYIQLDGASETKDVTYYNPSMGSSAGDMISTADDL
NKFFSYLLGGKLLKEQQLKQMLTTVPTGVGELGDSGLGIFKMKLSNGESI
WGHGGTIPGFLTFAGGTLGGKHTLAVNLNSLKADTPDPFKNILLAEFSK;
SEQ ID No. 8:
SSLQTNTQRDRTSVKQAMRDTLQLGYPGILAKTSEDGKTWGYAAGIADLR
TKKPMKTDFRFRIGSVTKTFTATVVLQLVGENRLKLDDYIEKWLPGVIQG
NGYDGNKITIREILNHTSGIAEYSRSKDVDFTDTKKSYTAEELVKMGISL
PPDFAPGKGWSYSNTGYVLLGILIEKVTGNSYAEEVENRIIEPLELSNTF
LPGNSSVIPGTNHARGYVQPDGASELKDVTYYNPSAGSSAGDMISTADDL
NKFFSYLLGGKLLKEQQLKQMLTTVPTGKEGIDGYGLGIYETKLPSGVSI
WGHSGGILGFTTLVGGKLGGKHTLVVNWNSLGRTGSPNPFKNILLAEFS
K;
SEQ ID No. 9:
NSSQEPNQKNRNGWKQVMQETIQIGAPGVLAKTSNKGKVNSYTAGVADLI
TKKPVKSDFRFRIGSVTKTFTATTVLQLVGENRVQLDDPIEKWLPGLVQG
NGYDGNQITIRQLLNHTSGIAEYLKSKDADVMNSKKTYTAEEIVKIGLSL
PPDFSPGKDWLYSNTGYVILGMLIEKITGNNYAEEIEKRIIEPLDLPNTF
LPGNSPVIPGKNHARGYVKMEETGELKDITYYNPSLANAAGDMISNADDL
NKFFSSLLGGKLLKERELKEMLTTVPVEGKGVGDGYGLGIYETKLPNGVS
VWGHGGSIPGFMTFAGGVIGGKHTFAVNVNSLGPVDILTQFDKMMQVEFN
K.
Disclosed are also the amino acid sequences from
which the above amino acid sequences with SEQ ID
NOs. 1-9 are derived, the respective sequences
with the starter amino acid M that can be removed
during post-translational modification (SEQ ID
NOs. 1A-9A), and the native amino acid sequences
from which the amino acid sequences with SEQ ID
Nos. 1-9 are derived, i.e., the amino acid
sequences with SEQ ID Nos. 1B-9B. The respective
sequences can also be used in the present
invention and are also encompassed according to
certain embodiments, respectively. For conciseness
sake, they are not repeated when discussing other
aspects of the invention, but are encompassed as
well as embodiments of the respective aspects
using or directed to the amino acid sequences
with SEQ ID NOs. 1-9.
SEQ ID No. 1A:
MASLQLSQTKELLTIRIQTDAVTKYGKEDAAIDAYLKGEGFSGMALVAKD
GGVLLRKGYGFAGTNKLNRPDSKTRIASITKSFTAASIMQLVEQGKLSLT
DPVSKFVTGIPRGDDITIHMLLSHTSGLQSEFTRSGDVTIEQTIEELRTK
QLKYEPGTTYLYSNNGYVLLAYVLEQLSGISYSDYVSEHFLTPLGMKNSG
TATPATPTIQGYILQKNNEWAAAPYYVSQSGTGTLYSTVDDLLKWDTALR
AGKVVSEQSLEAMYTPHSDKNYGYGWIALGLNGEKGVFHNGSGSGYATGM
LRNLDSGMTVILLGNHAGMDMTKLLQQVHKLAAEQ
SEQ ID No. 1B:
MTGIRGKKIAGWFNRHTVARGALIGIAAVSAFAGAGGAVAAAQPSSKVEA
AQPASSKVIAVEVNGKAVEWNVQPLIKDGTTFVPLREAGKAAAGNIEWDG
KTQTATIKVNGDVIVHRAGTSVVTVDGLAMNMSAPSLNIKGTLMLPLRSV
TDALKASLQLSQTKELLTIRIQTDAVTKYGKEDAAIDAYLKGEGFSGMAL
VAKDGGVLLRKGYGFAGTNKLNRPDSKTRIASITKSFTAASIMQLVEQGK
LSLTDPVSKFVTGIPRGDDITIHMLLSHTSGLQSEFTRSGDVTIEQTIEE
LRTKQLKYEPGTTYLYSNNGYVLLAYVLEQLSGISYSDYVSEHFLTPLGM
KNSGTATPATPTIQGYILQKNNEWAAAPYYVSQSGTGTLYSTVDDLLKWD
TALRAGKVVSEQSLEAMYTPHSDKNYGYGWIALGLNGEKGVFHNGSGSGY
ATGMLRNLDSGMTVILLGNHAGMDMTKLLQQVHKLAAEQ
SEQ ID No. 2A:
MTIRIQTDAVTKYGKEDAAIDAYLKGEGFSGMALVAKDGEVLLRKGYGFS
GTNKLNHPDAKSRIASLTKSFTAASIMQLVEQGKLSLTDPVSKFVTGIPR
GDDITIHMLLSHTSGLPSEFTRSGNVTIEQTIAELRTKQLKYEPGTTYLY
SNNGYVLLAYVLEQLSGESYADYVSEHFLTPLGMKNSGTATPATATIQGY
ILQKNNEWAAAPYYVSQSGTGTLYSTVDDLLKWDQALRAGKVVSKQSLEA
MYTPHSDKNYGYGWIAINLNGEKGVFHNGSGSGYATGMLRNLDSGMTVIL
LGNHAGMDMTKLLQQVHKLAAEQ
SEQ ID No. 2B:
MLNGFQVWQSCRFSFKRRRMRKRSWHGMTGIKGKKIAGWLNRHTVARSAL
IGIASVTAFAGVGGASVAAQTGSKGSKVEAVHPASSKAIAVEVNGKAVAW
NVQPLIKDDTTFVPLREAGKAAAGNIEWDGKTKTATIKVNGDVIVHQAGT
SVVTVDGLAMSMSAPSLNIKGTLMLPLRSVTDALKASLQLSQTKELLTIR
IQTDAVTKYGKEDAAIDAYLKGEGESGMALVAKDGEVLLRKGYGFSGTNK
LNHPDAKSRIASLTKSFTAASIMQLVEQGKLSLTDPVSKFVTGIPRGDDI
TIHMLLSHTSGLPSEFTRSGNVTIEQTIAELRTKQLKYEPGTTYLYSNNG
YVLLAYVLEQLSGESYADYVSEHFLTPLGMKNSGTATPATATIQGYILQK
NNEWAAAPYYVSQSGTGTLYSTVDDLLKWDQALRAGKVVSKQSLEAMYTP
HSDKNYGYGWIAINLNGEKGVFHNGSGSGYATGMLRNLDSGMTVILLGNH
AGMDMTKLLQQVHKLAAEQ
SEQ ID No. 3A:
MASLQLSQTKEFLTIRIQTDAVTKYGKEDAAIDAYLKGEGFSGMALVAKD
GEVLLRKGYGFSGTNKLNRPDAKSRIASITKSFTAASIMQLVEQGKLSLT
DPVSKFVTGIPRGDDITIHMLLSHTSGLPSEFTRSGDVTIEQTIAELRTK
QLKYEPGTTYLYSNNGYVLLAYVLEQLAGESYADYVNEHELTPLGMKNSG
TATPESPTIQGYILQKNNEWAAAPYYVSQSGTGTLYSTVDDLLKWDQALR
AGKVVSEQSLEAMYTPHSDKNYGYGWIAINLNGEKGVFHNGSGSGYATGM
LRNLDSGMTVILLGNHAGMDMTKLLQQVHKLAAEQ
SEQ ID No. 3B:
MTGIRGKKIAGWFNGHTVARSALIGMAAVTAFAGAGSAAAAAQPSSQVEA
VQPASSKAIAVEVNGKAVVWNVQPLIKDGTTFVPLREAGKAAAGNIEWDG
KTQTATIKVNGDVIVHPAGTSVITVDGLAMNMSAPSLNIKGTLMLPLRSV
TDALKASLQLSQTKEFLTIRIQTDAVTKYGKEDAAIDAYLKGEGFSGMAL
VAKDGEVLLRKGYGFSGTNKLNRPDAKSRIASITKSFTAASIMQLVEQGK
LSLTDPVSKFVTGIPRGDDITIHMLLSHTSGLPSEFTRSGDVTIEQTIAE
LRTKQLKYEPGTTYLYSNNGYVLLAYVLEQLAGESYADYVNEHELTPLGM
KNSGTATPESPTIQGYILQKNNEWAAAPYYVSQSGTGTLYSTVDDLLKWD
QALRAGKVVSEQSLEAMYTPHSDKNYGYGWIAINLNGEKGVFHNGSGSGY
ATGMLRNLDSGMTVILLGNHAGMDMTKLLQQVHKLAAEQ
SEQ ID No. 4A:
MSSVQTSTQRDRNSVKQAVRDTLQLGFPGILAKTSEGGKTWSYAAGVANL
SSKKPMKTDFRFRIGSVTKTFTATVVLQLAEENRLNLDDSIEKWLPGVIQ
GNGYDDKQITIRQLLNHTSGIAEYTRSKSFDLMDTKKSYRAEELVKMGIS
MPPDFAPGKSWSYSNTGYVLLGILIETVTGNSYAEEIENRIIEPLELSNT
FLPGNSSVIPGTKHARGYIQLDGASEPKDVTYYNPSMGSSAGDMISTADD
LNKFFSYLLGGKLLKEQQLKQMLTTVPTGEAALGRYGLGIYETKLPNGVS
IWGHGGSIPGFVTFAGGTLGGKHTLAVNLNSLNAESPDPFKNILLAEFSK
SEQ ID No. 4B:
MKTRSQITCASLALLIAGSSLLYTTQTLIVKAEPTQSVSSSVQTSTQRDR
NSVKQAVRDTLQLGFPGILAKTSEGGKTWSYAAGVANLSSKKPMKTDFRF
RIGSVTKTFTATVVLQLAEENRLNLDDSIEKWLPGVIQGNGYDDKQITIR
QLLNHTSGIAEYTRSKSFDLMDTKKSYRAEELVKMGISMPPDFAPGKSWS
YSNTGYVLLGILIETVTGNSYAEEIENRIIEPLELSNTFLPGNSSVIPGT
KHARGYIQLDGASEPKDVTYYNPSMGSSAGDMISTADDLNKFFSYLLGGK
LLKEQQLKQMLTTVPTGEAALGRYGLGIYETKLPNGVSIWGHGGSIPGFV
TFAGGTLGGKHTLAVNLNSLNAESPDPFKNILLAEFSK
SEQ ID No. 5A:
MSSLQTSTQSDRTSVKKAMRDELQLGYPGILAKISKGGKTWSYTAGVADL
KTKKPMKADFRFRIGSVTKTFIATVLLQLSGENRLNLDDSIEKWLPGVIQ
GNGYDGNQITIRQILNHTSGIADYVNSKDFDIMDTKKSYTAEEFVKMGIS
LPPDFAPGKGWSYSNTGYVLLGILIEKVTGNSYAEEVENRIIEPLDLSNT
FLPGNSSVIPGTKHARGYLQINGASELKDVTYINPGSSDGDMISTADDLN
KFFSCLLGGKLLKEQQLKQMLTTVPTNREGTGYGLGILEIKLPNGVSVWG
HRGAVPGFSTFAGGTLGGKHTLAINSNSLNLNNPEVFKNILLAEFRK
SEQ ID No. 5B:
KTRSKITCASLALLIGGSSLLYITPTSVVKAEPTQNVSSSLQTSTQSDRT
SVKKAMRDELQLGYPGILAKISKGGKTWSYTAGVADLKTKKPMKADFRFR
IGSVTKTFIATVLLQLSGENRLNLDDSIEKWLPGVIQGNGYDGNQITIRQ
ILNHTSGIADYVNSKDFDIMDTKKSYTAEEFVKMGISLPPDFAPGKGWSY
SNTGYVLLGILIEKVTGNSYAEEVENRIIEPLDLSNTFLPGNSSVIPGTK
HARGYLQINGASELKDVTYINPGSSDGDMISTADDLNKFFSCLLGGKLLK
EQQLKQMLTTVPTNREGTGYGLGILEIKLPNGVSVWGHRGAVPGFSTFAG
GTLGGKHTLAINSNSLNLNNPEVFKNILLAEFRK
SEQ ID No. 6A:
MSSLQTSTQSDRTSVKKAIRDELQLGYPGILAQISKGGKTWSYTAGIADL
RTKKPMKADFRFRIGSVTKTFIATVLLQLSGENRLNLDDSIEKWLPGVIQ
GNGYDGNQITIRQILNHTSGIADYINSKDFDIMDTKKSYTAEEFVKMGIS
LPPDFAPGKGWSYSNTGYVLLGILIEKVTGNSYAEEVENRIIEPLDLSNT
FLPGNSSVIPGTKHARGYLQLDGASELKDVTYINPGSSDGDMISTADDLN
KFFSYLLGGKLLKEQQLKQMLTTVPTNREGTGYGLGILEIKLPNGVSVWG
HRGGVLGFSTFAGGTLGGKHTLAINSNSFNINNPESFKNVLIAEFSK
SEQ ID No. 6B:
MKTRSKITCASLALLIGGSSLLYITPTSVVKAEPTQNVSSSLQTSTQSDR
TSVKKAIRDELQLGYPGILAQISKGGKTWSYTAGIADLRTKKPMKADFRF
RIGSVTKTFIATVLLQLSGENRLNLDDSIEKWLPGVIQGNGYDGNQITIR
QILNHTSGIADYINSKDFDIMDTKKSYTAEEFVKMGISLPPDFAPGKGWS
YSNTGYVLLGILIEKVTGNSYAEEVENRIIEPLDLSNTFLPGNSSVIPGT
KHARGYLQLDGASELKDVTYINPGSSDGDMISTADDLNKFFSYLLGGKLL
KEQQLKQMLTTVPTNREGTGYGLGILEIKLPNGVSVWGHRGGVLGFSTFA
GGTLGGKHTLAINSNSFNINNPESFKNVLIAEFSK
SEQ ID No. 7A:
MNSLQTSTQRDRNSVKEAMRDTLKLGYPGILAKTSEGGKTWSYAAGVADL
SNKKAMKTDFRFRIGSVTKTFTATVVLQLAGENRLNLDDSIEKWLPGVIQ
GNGYDDKQITIRQLLNHTSGIANYTRSKDFNMMDTKKSYTAEGLVKMGIS
MPPDFAPGKSWSYSNTGYVLLGILIEKVTGNSYAEEIENRIIEPLELANT
FLPGNSSVIPGTKHARGYIQLDGASETKDVTYYNPSMGSSAGDMISTADD
LNKFFSYLLGGKLLKEQQLKQMLTTVPTGVGELGDSGLGIFKMKLSNGES
IWGHGGTIPGFLTFAGGTLGGKHTLAVNLNSLKADTPDPFKNILLAEFSK
SEQ ID No. 7B:
MKKHSQITCTGLALLIIGGSLFYTTPTSSVKAESSIQNVSNSLQTSTQRD
RNSVKEAMRDTLKLGYPGILAKTSEGGKTWSYAAGVADLSNKKAMKTDFR
FRIGSVTKTFTATVVLQLAGENRLNLDDSIEKWLPGVIQGNGYDDKQITI
RQLLNHTSGIANYTRSKDENMMDTKKSYTAEGLVKMGISMPPDFAPGKSW
SYSNTGYVLLGILIEKVTGNSYAEEIENRIIEPLELANTFLPGNSSVIPG
TKHARGYIQLDGASETKDVTYYNPSMGSSAGDMISTADDLNKFFSYLLGG
KLLKEQQLKQMLTTVPTGVGELGDSGLGIFKMKLSNGESIWGHGGTIPGF
LTFAGGTLGGKHTLAVNLNSLKADTPDPFKNILLAEFSK
SEQ ID No. 8A:
MSSLQTNTQRDRTSVKQAMRDTLQLGYPGILAKTSEDGKTWGYAAGIADL
RTKKPMKTDFRFRIGSVTKTFTATVVLQLVGENRLKLDDYIEKWLPGVIQ
GNGYDGNKITIREILNHTSGIAEYSRSKDVDFTDTKKSYTAEELVKMGIS
LPPDFAPGKGWSYSNTGYVLLGILIEKVTGNSYAEEVENRIIEPLELSNT
FLPGNSSVIPGTNHARGYVQPDGASELKDVTYYNPSAGSSAGDMISTADD
LNKFFSYLLGGKLLKEQQLKQMLTTVPTGKEGIDGYGLGIYETKLPSGVS
IWGHSGGILGFTTLVGGKLGGKHTLVVNWNSLGRTGSPNPFKNILLAEFS
K
SEQ ID No. 8B:
MAQIVGIDRYLCTGALIYMREGFSLSLVRKSGTVSKHLYNMNRGEECLMK
IRSQITCASLALLIAGSSLLYTTPTSIVKAEPTQNVSSSLQTNTQRDRTS
VKQAMRDTLQLGYPGILAKTSEDGKTWGYAAGIADLRTKKPMKTDFRFRI
GSVTKTFTATVVLQLVGENRLKLDDYIEKWLPGVIQGNGYDGNKITIREI
LNHTSGIAEYSRSKDVDFTDTKKSYTAEELVKMGISLPPDFAPGKGWSYS
NTGYVLLGILIEKVTGNSYAEEVENRIIEPLELSNTFLPGNSSVIPGTNH
ARGYVQPDGASELKDVTYYNPSAGSSAGDMISTADDLNKFFSYLLGGKLL
KEQQLKQMLTTVPTGKEGIDGYGLGIYETKLPSGVSIWGHSGGILGFTTL
VGGKLGGKHTLVVNWNSLGRTGSPNPFKNILLAEFSK
SEQ ID No. 9A:
MNSSQEPNQKNRNGWKQVMQETIQIGAPGVLAKTSNKGKVNSYTAGVADL
ITKKPVKSDFRFRIGSVTKTFTATTVLQLVGENRVQLDDPIEKWLPGLVQ
GNGYDGNQITIRQLLNHTSGIAEYLKSKDADVMNSKKTYTAEEIVKIGLS
LPPDFSPGKDWLYSNTGYVILGMLIEKITGNNYAEEIEKRIIEPLDLPNT
FLPGNSPVIPGKNHARGYVKMEETGELKDITYYNPSLANAAGDMISNADD
LNKFFSSLLGGKLLKERELKEMLTTVPVEGKGVGDGYGLGIYETKLPNGV
SVWGHGGSIPGFMTFAGGVIGGKHTFAVNVNSLGPVDILTQFDKMMQVEF
NK
SEQ ID No. 9B:
MKTRNQIKLASLAVLLAGTTLSTPGFTVKAESTQNISNSSQEPNQKNRNG
WKQVMQETIQIGAPGVLAKTSNKGKVNSYTAGVADLITKKPVKSDFRFRI
GSVTKTFTATTVLQLVGENRVQLDDPIEKWLPGLVQGNGYDGNQITIRQL
LNHTSGIAEYLKSKDADVMNSKKTYTAEEIVKIGLSLPPDFSPGKDWLYS
NTGYVILGMLIEKITGNNYAEEIEKRIIEPLDLPNTFLPGNSPVIPGKNH
ARGYVKMEETGELKDITYYNPSLANAAGDMISNADDLNKFFSSLLGGKLL
KERELKEMLTTVPVEGKGVGDGYGLGIYETKLPNGVSVWGHGGSIPGFMT
FAGGVIGGKHTFAVNVNSLGPVDILTQFDKMMQVEFNK

According to certain embodiments, the D-stereospecific hydrolytic enzymes can catalyze the hydrolytic cleavage of at least one D-amino acid and/or D-amino acid derivative-protected functional group of a substrate to obtain a D-amino acid and/or a D-amino acid derivative and a D-amino acid and/or D-amino acid derivative at least partially deprotected substrate.

The method of the first aspect allows a selective deprotecting of an at least one D-amino acid and/or D-amino acid derivative-protected functional group of a substrate. The method of the first aspect further allows a rapidly and quantitatively cleavage of the D-amino acid and/or D-amino acid derivative from the D-amino acid and/or D-amino acid derivative-protected functional group of the substrate. The method allows a deprotecting without side reactions, under mild reaction conditions, and the method can be orthogonal to all previously established deprotecting and synthesis methods. Furthermore, this method can be fully applied even in the case of chemically labile substrate and target structures.

In a second aspect of the invention, a D-stereospecific hydrolytic enzyme is provided. The D-stereospecific hydrolytic enzyme is as characterized with regard to the method of the first aspect, to which reference is made here. The D-stereospecific hydrolytic enzyme of the second aspect is characterized by the following features:
at least one first 3₁₀-helix-2 structure comprising an amino acid sequence SXXK; at least one first α-helix structure comprising an amino acids sequence YSN; and at least one first β-sheet structure comprising an amino acid sequence HXG; wherein X is an amino acid selected from the group consisting of proteinogenic amino acids; wherein the first 3₁₀-helix-2 structure, the first α-helix structure, and the first β-sheet structure are arranged in close proximity. According to certain embodiments, the D-stereospecific hydrolytic enzyme has an amino acid sequence chosen from SEQ ID No. 1, No. 2 No. 3, No. 4, No. 5, No. 6, No. 7, No. 8 and No. 9, or has an amino acid sequence chosen from SEQ ID No. 1, No. 2 No. 3, No. 4, No. 5, No. 7, No. 8 and No. 9, as indicated above.

In a third aspect of the present invention, a use of the D-stereospecific hydrolytic enzyme for deprotecting of at least one D-amino acid and/or D-amino acid derivative-protected functional group of a substrate is provided. According to certain embodiments, the D-stereospecific hydrolytic enzyme is the D-stereospecific hydrolytic enzyme of the second aspect.

According to certain embodiments, the deprotecting can comprise the same steps and features of the deprotecting of at least one D-amino acid and/or D-amino acid derivative-protected functional group of a substrate described in the first aspect of the invention.

According to certain embodiments, the D-stereospecific hydrolytic enzyme can have an amino acid sequence chosen from SEQ ID No. 1, No. 2 No. 3, No. 4, No. 5, No. 6, No. 7, No. 8 and No. 9. According to certain embodiments, the D-stereospecific hydrolytic enzyme can have an amino acid sequence chosen from SEQ ID No. 1, No. 2 No. 3, No. 4, No. 5, No. 7, No. 8 and No. 9.

D-stereospecific hydrolytic enzymes, particularly the present D-stereospecific hydrolytic enzymes, are stringently regio-, chemo- and enantioselective for the respective recognized D-amino acid and/or D-amino derivative. Therefore, the use of the D-stereospecific hydrolytic enzymes allows a selective cleavage of at least one D-amino acid and/or D-amino acid derivative from a D-amino acid and/or D-amino acid derivative protected functional group of a substrate.

In a fourth aspect of the invention, a method of manufacturing a carbohydrate; a carbohydrate derivative; a nucleotide; an oligonucleotide; a polynucleotide; a nucleotide derivative; an oligonucleotide derivative; a polynucleotide derivative; an amino acid; a peptide; an oligopeptide; a polypeptide; a protein; a peptide intermediate; a peptide derivative; an oligopeptide derivative; a polypeptide derivative; or mixtures thereof, is provided, the method comprising:
Providing a precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, at least one D-amino acid and/or D-amino acid derivative, at least one first solvent, optionally at least one second solvent, optionally at least one third solvent, a D-stereospecific hydrolytic enzyme, and at least one first reagent;
Mixing the precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, the at least one first solvent and the at least one D-amino acid and/or D-amino acid derivative to obtain a first reaction mixture;
Reacting the precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof and the at least one D-amino acid and/or D-amino acid derivative to obtain a protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof;
Optionally removing the at least one first solvent from the first reaction mixture; Optionally reacting the protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, in one or more further reaction steps with one or more further reagents to obtain a reacted protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof;
Mixing the protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, or the reacted protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, the at least one first reagent and optionally the at least one second solvent to obtain a second reaction mixture;
Reacting the protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, or the reacted protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof and the at least one first reagent to obtain a protected carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof;
Optionally removing the at least one second solvent from the second reaction mixture;
Mixing the protected carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, optionally the at least one third solvent and the D-stereospecific hydrolytic enzyme to obtain a third reaction mixture; and Deprotecting the at least one D-amino acid and/or D-amino acid derivative from the protected carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof to obtain the carbohydrate; the carbohydrate derivative; the nucleotide; the oligonucleotide; polynucleotide; the nucleotide derivative; the oligonucleotide derivative; the polynucleotide derivative; the amino acid; the peptide; the oligopeptide; the polypeptide; the protein; the peptide intermediate; the peptide derivative; the oligopeptide derivative; the polypeptide derivative; or mixtures thereof.

The step of providing the different components is not particularly limited. According to certain embodiments, the components can be provided with or without at least one synthesis step before providing the different components. According to certain embodiments, the synthesis step can be selected from the group consisting of a chemical reaction, removing at least one of the components, and adding at least one component.

The steps of mixing different components are not particularly limited. The order in which the various components can be added in such mixing is also not particularly limited. The mixing is not particularly limited and any known mixing method can be used.

For the (optional) reaction steps, the reaction vessel is not particularly limited and any known reaction vessel can be used.

The optionally removing of the at least one first solvent and/or the at least second solvent is not particularly limited. For example, any known evaporation method can be used. According to certain embodiments, the at least one solvent and/or the at least second solvent can be removed by evaporation.

The step of reacting the precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof and the at least one D-amino acid and/or D-amino acid derivative to obtain a protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof is not particularly limited. According to certain embodiments, the functional group of the precursor can be selected from the group consisting of an amino group, a carboxylic group, a hydroxyl group, a sulfhydryl group, and a selenohydril group. According to certain embodiments, the functional group can be an amino group, a hydroxyl group, a sulfhydryl group and/or a selenohydril group and the D-amino acid and/or D-amino acid derivative can bind via the carbonyl group to the substrate.

The step of deprotecting can comprise the same steps and features as the deprotecting of at least one D-amino acid and/or D-amino acid derivative-protected functional group of a substrate described in the method of the first aspect of the invention. According to certain embodiments, the step of deprotecting can further comprise adding a buffer to the reaction mixture, to obtain the desired pH value of the reaction mixture. The buffer is not particularly limited. According to certain embodiments, the buffer can be selected from the buffers described in the first aspect of the invention. According to certain embodiments, the step of deprotecting can further comprise adding one or more additives to the reaction mixture. The additive is not particularly limited as long as it is compatible with the compounds present in the reaction mixture. According to certain embodiments, the additive can be selected from the additives described in the first aspect of the invention

The carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof are not particularly limited and can bet the same described in the first aspect of the invention.

The at least one first solvent, the at least second solvent, and the at least third solvent are not particularly limited. They can be the same or different but are, according to certain embodiments, different and can be the same as the at least one solvent described in the first aspect of the invention.

Further, the D-amino acid and the D-amino acid derivative are not particularly limited and can be selected from the D-amino acid and the D-amino acid derivatives described in the first aspect of the invention.

The D-stereospecific hydrolytic enzyme is also not limited and can be characterized by the same features as the D-stereospecific hydrolytic enzyme described in the first and the second aspect of the invention.

The at least one first reagent is not particularly limited. According to certain embodiments, the at least one first reagent can be selected from the group consisting of a precursor to introduce a chemical group; a base; an acid; a reducing agent; an oxidizing agent; a carbohydrate; a carbohydrate derivative; a nucleotide; a oligonucleotide; a polynucleotide; a nucleotide derivative; an oligonucleotide derivative; a polynucleotide derivative; an amino acid; a peptide; an oligopeptide; a polypeptide; a protein; a peptide intermediate; a peptide derivative; an oligopeptide derivative; a polypeptide derivative; or mixtures thereof.

The precursor to introduce a chemical group is not particularly limited.

The chemical group can be selected from the group consisting of a hydrogen, a halogen, an alkyl group, an alkenyl group, an alkinyl group, an aromatic group, an ester group, a carboxylic group, an acetyl group, an ether group, a keto group, an aldehyde group, an amine group, an amide group, an imine group, an imide group, an acetylamino group, an acetal group, a hemiacetal group, a nitrile group, a thiol group, a sulfide group, and a posphine group

The base is not particularly limited. According to certain embodiments, the base can be selected from the group consisting of an organic and inorganic base. According to certain embodiments, the base can be selected from the group consisting of amines, carbonates, carbamates, hydroxides, oxides, and ammonia, e.g. amines, carbonates, hydroxides, and ammonia.

The acid is not particularly limited. According to certain embodiments, the acid can be selected from the group consisting of an organic and inorganic acid. According to certain embodiments, the acid can be selected from the group consisting of a carboxylic acid, sulfuric acid, hydrochloric acid, phosphoric acid, and nitric acid. The carboxylic acid can be selected from the group consisting of formic acid, and acetic acid.

The reducing agent is not particularly limited. According to certain embodiments, the reducing agent can be selected from the group consisting of metals like sodium or lithium, hydrogen, lithium aluminium hydride, Red-Al (NaAlH₂(OCH₂CH₂OCH₃)₂), diborane, sodium borohydride, Fe compounds that including a Fe²⁺, such as iron(II) sulfate, Sn compounds including a Sn²⁺ ion, such as tin(II) chloride, sulfites, dithionates, thiosulfates, hydrazine, diisobutylaluminium hydride (DIBAL-H), oxalic acid, formic acid, ascorbic acid, reducing sugars, such as erythrose, phosphites, hypophosphites, DTT (dithiothreitol), carbon monoxide, and tris-2-carboxyethylphosphine hydrochloride (TCEP).

The oxidizing agent is not particularly limited. According to certain embodiments, the oxidizing agent can be selected from the group consisting of oxygen, nitrogen oxides, peroxides like hydrogen peroxide, hydrogen peroxide derivatives, manganates, chromates like PCC (pyridinium chlorochromate) or PDC (pyridinium dichromate), bromates, chlorites, and TEMPO (2,2,6,6-tetramethylpiperidin-1-yl)oxyl or (2,2,6,6-tetramethylpiperidin-1-yl)oxidanyl).

The carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof are not particularly limited and can be as described in the first aspect of the invention.

This manufacturing method allows modeling the biological activity or biochemical functionality of a compound during the synthesis of the compound. For example, by introducing one or more functionality, posttranslational modification and/or artificial derivatisation to the D-amino acid resulting in a D-amino acid derivative, the chemical and physical properties can be changed like solubility or compatibility with other compounds as it was described above. This includes therapeutics that can be modified by D-amino acids and/or D-amino acid derivatives and lead to the inactivation of this compound resulting in a prodrug. As a consequence, therapeutics can be introduced as inactive precursor and, after accumulation in tissue, an activation by cleavage of the D-amino acid and/or D-amino acid derivative can be carried out.

D-amino acids and/or D-amino acid derived functionalities further can block catalytically active residues or prevent structure-inducing interactions. This enables hindering formation of a structural element or an interaction of different amino acid side chains, thus being able to regulate activity of an enzyme or binding of an interaction partner to a target structure. After cleaving in the D-amino acid and/or D-amino acid derivative formation of the active protein species or the intended interaction can occur.

By using the D-stereospecific hydrolytic enzymes the at least one D-amino acid and/or D-amino acid derivative protected functional group of a substrate can be easily cleaved at any time of the synthesis procedure of a compound. That means, that the at least one D-amino acid and/or D-amino acid derivative can be introduced to the substrate at any time during the synthesis of a chemical compound and can cleaved at any time after introducing it. Hence, the chemical and physical properties of the substrate can be precisely, selectively and effectively adapted depending on the required reaction conditions and the required reaction system at a certain time of the synthesis of a chemical compound.

In a fifth aspect of the invention, the use of a D-amino acid and/or D-amino acid derivative as a protecting group for a functional group of a substrate is provided.

The D-amino acid, the D-amino acid derivative, the functional group and the substrate are not particularly limited and can be as described above.

The above embodiments can be combined arbitrarily, if appropriate. Further embodiments and implementations of the invention also comprise not explicitly cited combinations of features mentioned beforehand or hereinafter with regard to examples of the invention. Particularly, a skilled person will also add single aspects as improvements or supplements to the respective basic form of the present invention.

### EXAMPLES

### Preparation and Characterization of D-Stereospecific Hydrolytic Enzymes

The preparation of D-stereospecific hydrolytic enzymes with SEQ ID No. 1, No. 2, No. 3, No. 4, No, 5, No. 6, No. 7, No. 8, and No. 9 is performed under standard conditions (growth conditions: LB-media, Kanamycin, 37° C., continuous shaking; protein biosynthesis induction: 0.1 M IPTG and temperature shift to 30° C.) in E. coli.

The isolated yields of these D-stereospecific hydrolytic enzymes after expression in E. coli and subsequent purification by ion exchange (SP-Sepharose or Supelco 650 M, 0.1 M HEPES, 0.1 M NaCl, pH 7.0 (SEQ ID No. 1, No. 2, No. 3, i.e. Paenidases 6.0); protein elution: 0.1 M TRIS pH 9.0) and final gel permeation chromatography (HiLoad 16/60 Superdex 75 prep grade column, 0.1 M HEPES, 0.01 M NaCl, pH 8.0) are shown in Table 1.

TABLE 1

| D-stereospecific hydrolytic enzyme | Yield (mg/liter culture) |
|---|---|
| SEQ ID No. 6 | 7.0 |
| SEQ ID No. 7 | 11.0 |
| SEQ ID No. 8 | 4.3 |
| SEQ ID No. 9 | 6.3 |
| SEQ ID No. 4 | 5.0 |
| SEQ ID No. 1 | 5.0 |
| SEQ ID No. 2 | 5.0 |
| SEQ ID No. 3 | 5.0 |
| SEQ ID No. 5 | 18.5 |

### Specific Activity as of D-Stereospecific Hydrolytic Enzyme

Specific activities Aₛ (s⁻¹) of D-stereospecific hydrolytic enzymes with SEQ ID No. 4, No. 6, No. 7, No. 8, and No. 9 towards a selected D-amino acid derivative, that was esterified, were tested.

In these experiments, a NaPi buffer with a concentration of 0.1 M based on the reaction mixture was used. The pH value of the reaction mixture was 8.0. As an Example, the substrate Bz-D-Phe-OMe (benzoyl-D-phenylalanine-methyl ester) was used. As Comparative Examples, the substrates Bz-L-Phe-OMe (benzoyl-L-phenylalanine-methyl ester) and Bz-Gly-OGp (benzoyl-glycyl-4-guanidinophenyl ester) was used. The concentration of the substrate was 2 mM. The concentration of the enzyme was in the range between 2-400 nM. The concentration are based on the reaction mixture. Water including 1% DMF (v/v) was used as a solvent. The activity measurement was a discontinous measurement via UPLC (gradient 0% B=>60% B (v/v), eluents: A water (0.1% (v/v) trifluoracetic acid), B acetonitrile (0.1% (v/v) trifluoracetic acid)) at a flow rate of 0.5 ml/min and a detection wavelength of 254 nm at 40° C. 25 μl sample volumes were taken at specific timepoints (0 to 5 min) and quenched with 25 μl 50% (v/v) acetic acid. The Aₛ is defined as the convertion of μmol substrate per time scale per μmol enzyme and given as kat/mol.

In this case, the D-stereospecific hydrolytic enzymes catalyze the hydrolysis of the respective ester bonds resulting in an alcohol group and a carboxylic group. The specific activity Aₛ refers to the rate at which the specific D-stereospecific hydrolytic enzymes catalyze the hydrolysis of the respective ester bond.

The specific activities Aₛ of D-stereospecific hydrolytic enzymes with SEQ ID No. 4, No. 6, No. 7, No. 8, and No. 9 are shown in Table 2.

TABLE 2

| | Specific activity Aₛ (kat/mol) | | |
|---|---|---|---|
| D-stereospecific | | Comparative | Comparative |
| hydrolytic | Example: | Example: | Example: |
| enzyme | Bz-D-Phe-OMe | Bz-L-Phe-OMe | Bz-Gly-OGp |
| SEQ ID No. 6 | 31 | 0.003 | 12.1 |
| SEQ ID No. 7 | 60 | 0.100 | 6.3 |
| SEQ ID No. 8 | 89 | 0.441 | 2.5 |
| SEQ ID No. 9 | 138 | 0.332 | 2.0 |
| SEQ ID No. 4 | 36 | n.d. | 1.8 |

n.d. = not detectable

As can be seen in Table 2, the tested D-stereospecific hydrolytic enzymes exhibit a high Aₛ when a D-amino acid-containing substrate is used and a low Aₛ when a L-amino acid-containing substrate is used. Thus, the tested D-stereospecific hydrolytic enzymes selectively recognize and convert D-amino acids or D-amino acid derivatives.

In further experiments, specific activities Aₛ (mkat/mol) of D-stereospecific hydrolytic enzymes with SEQ ID No. 2, No. 4, No. 6, No. 7, No. 8, and No. 9 towards selected D-amino acid derivative protected peptides were tested. In this case, the N-terminus of the peptide is protected by the D-amino acid derivative.

In these experiments with the conditions otherwise as above, a NaPi buffer with a concentration of 0.1 M based on the reaction mixture was used. The pH value of the reaction mixture was 8.0. The peptide had an amino acid sequence of AKAKY(NO₂) (SEQ ID No. 10), wherein Y was modified with a NO₂ group to result in 3-nitrotyrosine. The concentration of the D-amino acid derivative-protected peptide was 100 μM based on the reaction mixture. In addition, DTT was added in a concentration of 10 mM based on the reaction mixture. Furthermore, NaCl is added in a concentration of 150 mM based on the reaction mixture. The concentration of the respective D-stereospecific hydrolytic enzyme was 1 nM to 1 μM based on the reaction mixture. As D-amino acid derivatives, the D-amino acid derivatives shown in Table 3 below are used. The reaction temperature was 30° C. Water was used as solvent.

The evaluation was done with a discontinous measurement via UPLC (gradient 0% B=>20% or 40% or 60% B (v/v), eluents: A water (0.1% (v/v) trifluoracetic acid), B acetonitrile (0.1% (v/v) trifluoracetic acid)) at a flow rate of 0.5 ml/min and a detection wavelength of 360 nm at 40° C. 25 μl sample volumes were taken at specific timepoints (0 to 5 min) and quenched with 25 μl 50% (v/v) acetic acid. All values represent the average of a duplicate determination. The deviation was always below 10%.

In this case, the D-stereospecific hydrolytic enzymes catalyze the hydrolysis of the respective carboxamide bond between the D-amino acid derivative and the peptide resulting in an amine group and a carboxylic group, here e.g. with f being D-Phe (source: IUPAC-IUB Commission on Biochemical Nomenclature (CBN), A One-Letter Notation for Amino Acid Sequences, 1968, Arch. Biochem. Biophys. 125(3), i-v (1968)). The specific activity Aₛ refers to the rate at which the specific D-stereospecific hydrolytic enzyme catalyzes the hydrolysis of the respective amide or carboxamide bond.

The specific activities Aₛ of the D-stereospecific hydrolytic enzymes with SEQ ID No. 2, No. 4, No. 6, No. 7, No. 8, and No. 9 towards selected D-amino acid derivative-protected peptides are shown in Table 3. In this regard it is to be noted that the (Bz-f) in AAK(Bz-f)AKY(NO₂) (SEQ ID No. 18) is located on the NH₂ of the chain of lysine and not inside the peptide chain, so that the resulting peptide chain after cleavage therof is AAKAKY(NO₂) (SEQ ID No. 137), i.e the same as after cleavage of Bz-fAAKAKY(NO₂) (SEQ ID No. 21). From Bz-fCAKAKY(NO₂) (SEQ ID No. 11), Z-fCAKAKY(NO₂) (SEQ ID No. 12), Suc-fCAKAKY(N02) (SEQ ID No. 13) Ac-fCAKAKY(NO₂) (SEQ ID No. 14), Boc-fCAKAKY(NO₂) (SEQ ID No. 15), Abz-fCAKAKY(NO₂) (SEQ ID No. 16) Fmoc-fCAKAKY(N02) (SEQ ID No. 17), fCAKAKY(NO₂) and (SEQ ID No. 19), the resulting product is CAKAKY(NO₂) (SEQ ID No. 169). The other products after cleavage of the protected D-Phe from SEQ ID No. 20 to 38 are: GAKAKY(NO₂) (SEQ ID No. 136), AAKAKY(NO₂) (SEQ ID No. 137), SAKAKY(NO₂) (SEQ ID No. 138), TAKAKY(NO₂) (SEQ ID No. 139), LAKAKY(NO₂) (SEQ ID No. 140), VAKAKY(NO₂) (SEQ ID No. 141), IAKAKY(NO₂) (SEQ ID No. 142), FAKAKY(NO₂) (SEQ ID No. 143), YAKAKY(NO₂) (SEQ ID No. 144), WAKAKY(NO₂) (SEQ ID No. 145), RAKAKY(NO₂) (SEQ ID No. 146), KAKAKY(NO₂) (SEQ ID No. 147), HAKAKY(NO₂) (SEQ ID No. 148), DAKAKY(NO₂) (SEQ ID No. 149), EAKAKY(NO₂) (SEQ ID No. 150), QAKAKY(NO₂) (SEQ ID No. 151), NAKAKY(NO₂) (SEQ ID No. 152), MAKAKY(NO₂) (SEQ ID No. 153), PAKAKY(NO₂) (SEQ ID No. 154).

TABLE 3

| | As (mkat/mol) | | | | | |
|---|---|---|---|---|---|---|
| | D-stereospecific hydrolytic enzyme | | | | | |
| | SEQ | SEQ | SEQ | SEQ | SEQ | SEQ |
| | ID | ID | ID | ID | ID | ID |
| Substrate | No. 9 | No. 7 | No. 8 | No. 4 | No. 6 | No. 2 |
| Bz-fCAKAKY(NO₂) (SEQ ID No. 11) | 141500 | 10200 | 2900 | 14200 | 1.5 | — |
| Z-fCAKAKY(NO₂) (SEQ ID No. 12) | 6000 | 900 | 980 | 4600 | — | — |
| Suc-fCAKAKY(NO₂) (SEQ ID No. 13) | 2200 | 48 | 87 | 180 | n.d. | 1 |
| Ac-fCAKAKY(NO₂) (SEQ ID No. 14) | 500 | 130 | 130 | 420 | — | — |
| Boc-fCAKAKY(NO₂) (SEQ ID No. 15) | 1200 | — | — | — | — | — |
| Abz-fCAKAKY(NO₂) (SEQ ID No. 16) | 80000 | 1660 | 2220 | 3100 | — | — |
| Fmoc-fCAKAKY(NO₂) (SEQ ID No. 17) | 300 | 500 | 150 | 470 | — | — |
| AAK(Bz-f)AKY(NO₂) (SEQ ID No. 18) | 14200 | 2330 | 190 | 650 | n.d. | — |
| fCAKAKY(NO₂) (SEQ ID No. 19) | 5 | <0.008 | 5 | 9 | — | — |
| Bz-fGAKAKY(NO₂) (SEQ ID No. 20) | 51000 | 760 | 500 | 3000 | n.d. | 6 |
| Bz-fAAKAKY(NO₂) (SEQ ID No. 21) | 36200 | 330 | 480 | 2400 | — | — |
| Bz-fSAKAKY(NO₂) (SEQ ID No. 22) | 88350 | 2100 | 920 | 6900 | — | — |
| Bz-fTAKAKY(NO₂) (SEQ ID No. 23) | 121000 | 910 | 1030 | 7500 | — | — |
| Bz-fLAKAKY(NO₂) (SEQ ID No. 24) | 183200 | 900 | 1710 | 12800 | — | — |
| Bz-fVAKAKY(NO₂) (SEQ ID No. 25) | 165800 | 1900 | 1200 | 11900 | — | — |
| Bz-fIAKAKY(NO₂) (SEQ ID No. 26) | 249000 | 920 | 1650 | 14200 | — | — |
| Bz-fFAKAKY(NO₂) (SEQ ID No. 27) | 890 | 12500 | 2400 | 56000 | — | 34 |
| Bz-fYAKAKY(NO₂) (SEQ ID No. 28) | 1780 | 27700 | 1800 | 37000 | — | 7 |
| Bz-fWAKAKY(NO₂) (SEQ ID No. 29) | 1070 | 182000 | 2800 | 11300 | — | 15 |
| Bz-fRAKAKY(NO₂) (SEQ ID No. 30) | 46600 | 910 | 450 | 10200 | — | — |
| Bz-fKAKAKY(NO₂) (SEQ ID No. 31) | 59600 | 180 | 460 | 4900 | — | — |
| Bz-fHAKAKY(NO₂) (SEQ ID No. 32) | 158000 | 8300 | 1600 | 19600 | — | — |
| Bz-fDAKAKY(NO₂) (SEQ ID No. 33) | 32000 | 1360 | 320 | 4000 | — | — |
| Bz-fEAKAKY(NO₂) (SEQ ID No. 34) | 980 | 40 | 65 | 2040 | — | — |
| Bz-fQAKAKY(NO₂) (SEQ ID No. 35) | 93060 | 1700 | 1300 | 5000 | — | — |
| Bz-fNAKAKY(NO₂) (SEQ ID No. 36) | 147600 | 5000 | 1400 | 7700 | — | — |
| Bz-fMAKAKY(NO₂) (SEQ ID No. 37) | 978 | 3000 | 3200 | 34500 | — | — |
| Bz-fPAKAKY(NO₂) (SEQ ID No. 38) | 0.5 | <0.008 | <0.005 | n.d. | — | — |

n.d. = not detectable

As can be seen from Table 3, all tested D-stereospecific enzymes are active regarding the selected substrates. Hence, the D-stereospecific enzymes are able to convert peptides having at least one functional group protected with a D-amino acid or D-amino acid derivative. This is even the case if no protective group is present.

In further experiments, the specific activities Aₛ (mkat/mol) of D-stereospecific hydrolytic enzymes with SEQ ID No. 4, No. 7, No. 8, and No. 9 towards selected D-amino acid derivative protected peptides were tested as described above, except that the concentration of the D-amino acid derivative protected peptide was 1 mM based on the reaction mixture. The results are shown in Table 4. All values represent the average of a duplicate determination. The deviation was always below 10%.

TABLE 4

| | As (mkat/mol) | | | |
|---|---|---|---|---|
| | D-stereospecific | | | |
| | hydrolytic enzyme | | | |
| | SEQ ID | SEQ ID | SEQ ID | SEQ ID |
| Substrate | No. 9 | No. 7 | No. 8 | No. 4 |
| Bz-fCAKAKY(NO₂) | 167000 | 33000 | 4200 | 28800 |
| Z-fCAKAKY(NO₂) | 6100 | 900 | 1700 | 4700 |
| Suc-fCAKAKY(NO₂) | 18000 | 540 | 780 | 2400 |
| Ac-fCAKAKY(NO₂) | 300 | 1100 | 290 | 6900 |
| Boc-fCAKAKY(NO₂) | 1600 | 5800 | — | — |
| Abz-fCAKAKY(NO₂) | 90000 | 4100 | 3800 | 4800 |
| Fmoc-fCAKAKY(NO₂) | n.s.* | n.s.* | n.s.* | n.s.* |
| AAK(Bz-f)AKY(NO₂) | 24400 | 2830 | 370 | 3900 |
| fCAKAKY(NO₂) | 27 | 4 | 75 | 60 |
| Bz-fGAKAKY(NO₂) | 76000 | 5500 | 930 | 10100 |

n.s. = not soluble
*can be cleaved at higher solvent ratio

As can be seen from Table 4, the concentration of the applied substrate can be varied without deterioration of the activity of the D-stereospecific hydrolytic enzymes. Thus, the D-stereospecific hydrolytic enzymes can be applied in various synthesis conditions.

### Subsite Mapping Experiments Regarding the S1′-Subsite

Subsite mapping experiments were performed to characterize the S1′-binding pocket. For this purpose Bz-Gly-OGp and Bz-D-Phe-OMe were used as acyl donors and selected D- and L-amino acids amides (X/xₐₐ-NH₂, x represents a D-amino acid, whereas X represents an L-amino acid) were used as acyl acceptors. Activity measurement are carried out with a discontinous measurement via HPLC (gradient 0% B=>20% or 40% or 60% B (v/v), eluents: A water (0.1% (v/v) trifluoracetic acid), B acetonitrile (0.1% (v/v) trifluoracetic acid)) at a flow rate of 0.5 ml/min and detection wavelengths of 220 nm and 254 nm, respectively, at 40° C. 25 μl sample volumes were taken at specific timepoints (0 to 5 min) and quenched with 25 μl 50% (v/v) acetic acid. In the experiments, a NaPi buffer with a concentration of 0.1 M based on the reaction mixture was used. The pH value of the reaction mixture was 8.0. The concentration of the acyl acceptor was 10 mM (amino acid amide or pentapeptide) based on the reaction mixture. The concentration of the acyl donor (Bz-Gly-OGp, or Bz-Phe-OMe) was 1 mM based on the reaction mixture. The concentration of the D-stereo-hydrolytic enzyme was 500 nM. Water including 1% DMF (v/v) was used as a solvent.

The respective model reactions (aminolysis reaction) are shown below (Formulas 1 and 2). In Formulas 1 and 2 DHy represents the respective D-stereospecific hydrolytic enzyme as described below.

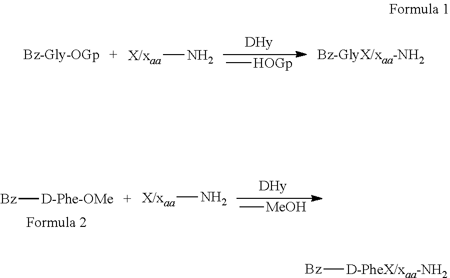

The reciprocal partition value is used to determine the efficiency of such reactions (Formula 3) (source: Schellenberger V, Turck C W, Rutter W J. Role of the S′ subsites in serine protease catalysis. Active-site mapping of rat chymotrypsin, rat trypsin, alpha-lytic protease, and cercarial protease from Schistosoma mansoni. Biochemistry. 1994 Apr. 12; 33(14):4251-7. doi: 10.1021/bi00180a020. PMID: 8155642):

$$p = [ N ] \frac{{v}_{H}}{{v}_{A}} \qquad ( Formula 3 )$$

$$p : Partition value$$

$$[ N ] : total nucleophile concentration$$

$${v}_{H} : Reaction rate hydrolysis$$

$${v}_{A} : Reaction rate aminolysis$$

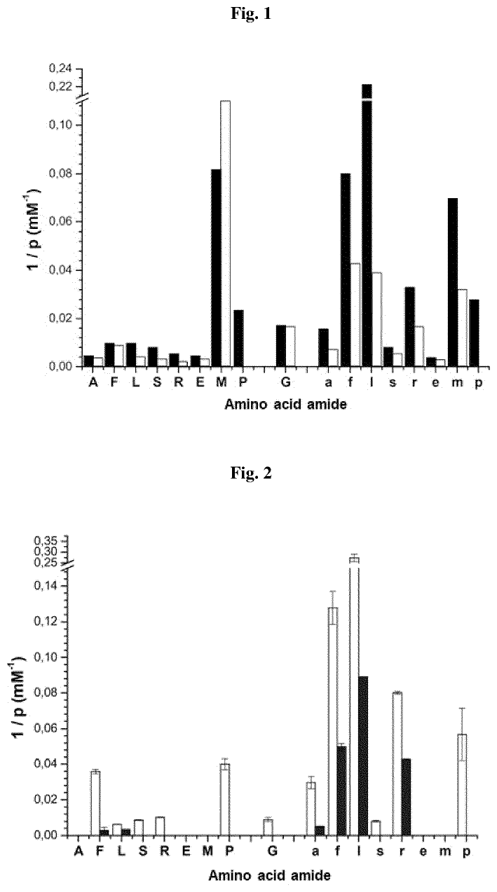

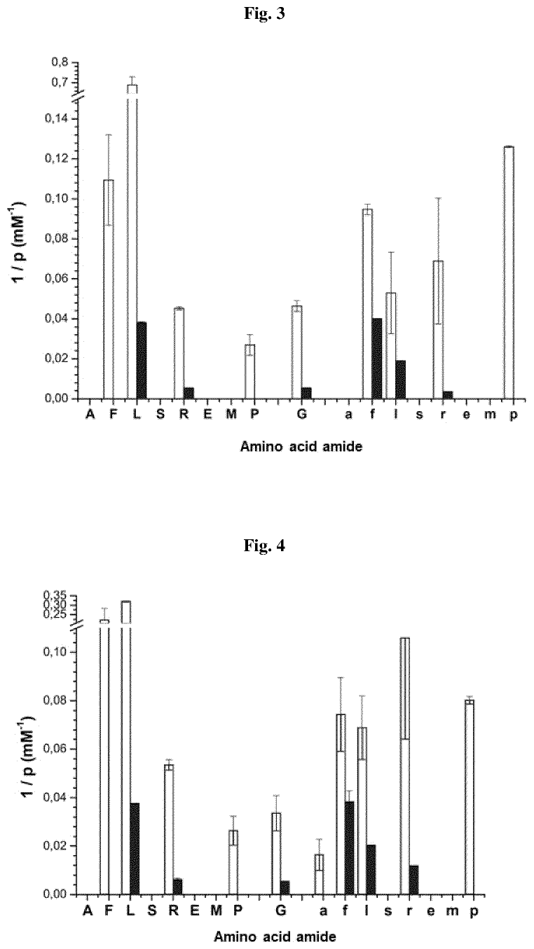

The reciprocal partition value (1/p) of S1′ subsite mapping of the D-stereospecific hydrolytic enzyme with SEQ ID No. 6 is shown in FIG. 1. The reciprocal partition value (1/p) of S1′ subsite mapping of the D-stereospecific hydrolytic enzyme with SEQ ID No. 7 is shown in FIG. 2. The reciprocal partition value (1/p) of S1′ subsite mapping of the D-stereospecific hydrolytic enzyme with SEQ ID No. 8 is shown in FIG. 3. The reciprocal partition value (1/p) of S1′ subsite mapping of the D-stereospecific hydrolytic enzyme with SEQ ID No. 9 is shown in FIG. 4.

In FIG. 1 to 4 the black bars refer to Bz-D-Phe-OMe and the white bars to Bz-Gly-OGp.

The smaller the reciprocal value (1/p) the higher is the reaction rate of the hydrolysis reaction and the higher is the rate of the aminolysis reaction, reflecting the S1′ specificity.

As it can be seen from FIG. 1 to 4, the aminolysis reaction using a D-amino acid as xₐₐ in the respective amide (xₐₐ —NH₂, D-amino acid in P1′ position) is preferred as lip gets larger for all tested D-stereospecific enzymes. Hence, the D-stereospecific enzymes recognize specifically the D-amino acid or the D-amino acid derivative when the D-amino acid or D-amino acid is in P1′ position. The 1/p value mainly decreases when using L-amino acids (Xₐₐ), which underlines the high S′1 subsite selectivity of the D-stereospecific hydrolytic enzymes towards the hydrolysis of D-amino acid or D-amino acid derivative-protected functional groups of substrates, particularly in case of single amino acids or derivatives thereof.

A similar experiment was carried out using an all-D/L peptide (xₐₐaaaGly, /Xₐₐ AAAG) as acyl acceptor instead of the D/-L amino acid amides above. The varied D-/L-amino acid is in P1′ position. Bz-Gly-OGp was used as acyl donor again. The respective model reaction (hydrolysis reaction) is shown below (Formula 4A and 4 B). In Formula 4 DHy represents a D-stereospecific hydrolytic enzyme.

In the experiments, a NaPi buffer with a concentration of 0.1 M based on the reaction mixture was used. The pH value of the reaction mixture was 8.0. The concentration of the acyl acceptor was 10 mM based on the reaction mixture. The concentration of the acyl donor was 1 mM based on the reaction mixture. The concentration of the D-stereo-hydrolytic enzyme was 500 nM. Water including 1% DMF was used as a solvent.

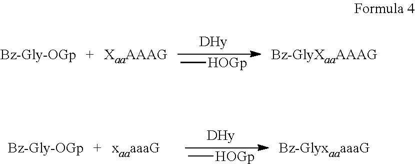

The following SEQ ID apply: XAAAG (SEQ ID No. 106), xaaaG (SEQ ID No. 107), Bz-GXAAAG (SEQ ID No. 170), Bz-GxaaaG (SEQ ID No. 171).

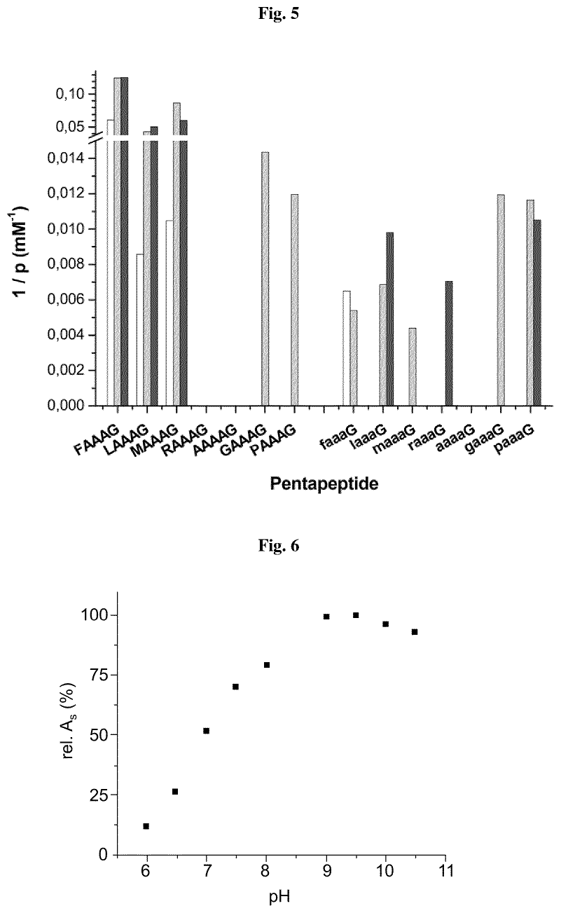

The reciprocal partition value (1/p) of S1′ subsite mapping of the D-stereospecific hydrolytic enzymes with SEQ ID No. 7 (white bars), 8 (light grey bars) and 9 (dark grey bars) are shown in FIG. 5. The following sequences are noted as educts and products, respectively: FAAAG (SEQ ID No. 108), faaaG (SEQ ID No. 109), Bz-GFAAAG (SEQ ID No. 110), Bz-GfaaaG (SEQ ID No. 111), LAAAG (SEQ ID No. 112), laaaG (SEQ ID No. 113), Bz-GLAAAG (SEQ ID No. 114), Bz-GlaaaG (SEQ ID No. 115), MAAAG (SEQ ID No. 116), maaaG (SEQ ID No. 117), Bz-GMAAAG (SEQ ID No. 118), Bz-GmaaaG (SEQ ID No. 119), RAAAG (SEQ ID No. 120), raaaG (SEQ ID No. 121), Bz-GRAAAG (SEQ ID No. 122), Bz-GraaaG (SEQ ID No. 123), AAAAG (SEQ ID No. 124), aaaaG (SEQ ID No. 125), Bz-GAAAAG (SEQ ID No. 126), Bz-GaaaaG (SEQ ID No. 127), GAAAG (SEQ ID No. 128), gaaaG (SEQ ID No. 129), Bz-GGAAAG (SEQ ID No. 130), Bz-GgaaaG (SEQ ID No. 131), PAAAG (SEQ ID No. 132), paaaG (SEQ ID No. 133), Bz-GPAAAG (SEQ ID No. 134), Bz-GpaaaG (SEQ ID No. 135).

In contrast to single amino acid derivatives the D-stereospecific enzymes recognize specifically the L-amino acid derivative in P1′ position when a peptide is used as a substrate. This makes the enzymes to cleave D-L amino acid bonds in case of longer peptides.

### Experiments Using Fluorogenic Substrates

In further experiments, the hydrolysis reaction using D-stereospecific hydrolytic enzymes with SEQ ID No. 4, No. 6, No. 7, No. 8, No. 9 was carried out with internally quenched fluorogenic substrates (IQFS) (Abz-AAX/xₐₐFAAK(DNP)-OH) (Abz=2-aminobenzoic acid, DNP=2,4-dinitrophenol). By cleavage of the IQFS, a fluorescence signal is released and can be monitored by continuous fluorescence measurement (0.1 M NaPi, pH 8.0, 1% DMSO (v/v), 5-1000 nM enzyme, excitation wavelength 320 nm emission wavelength 420 nm, reaction volume 100 μl, 96-well micro titer plate, time scale 2 h). The respective model reaction is shown below (Formula 5). In Formula 5 DHy represents the respective D-stereospecific hydrolytic enzyme.

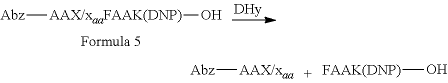

The following SEQ ID apply: Abz-AAXFAAK(DNP)-OH (SEQ ID No. 172), Abz-AAxFAAK(DNP)-OH (SEQ ID No. 173), FAAK(DNP)-OH (SEQ ID No. 155).

The apparent specific activities (A_{s,app}) are shown in Table 5. In Table 5 Values for the D-configured amino acid (xₐₐ) are on the left, and for the L-configured amino acid (Xₐₐ) are on the right. The sequences in Table 5 are as follows: AAFFAAK (SEQ ID No. 74), AAfFAAK (SEQ ID No. 75), AAWFAAK (SEQ ID No. 76), AAwFAAK (SEQ ID No. 77), AAYFAAK (SEQ ID No. 78), AAyFAAK (SEQ ID No. 79), AAAFAAK (SEQ ID No. 80), AAaFAAK (SEQ ID No. 81), AAVFAAK (SEQ ID No. 82), AAvFAAK (SEQ ID No. 83), AALFAAK (SEQ ID No. 84), AAlFAAK (SEQ ID No. 85), AASFAAK (SEQ ID No. 86), AAsFAAK (SEQ ID No. 87), AAMFAAK (SEQ ID No. 88), AAmFAAK (SEQ ID No. 89), AAHFAAK (SEQ ID No. 90), AAhFAAK (SEQ ID No. 91), AAKFAAK (SEQ ID No. 92), AAkFAAK (SEQ ID No. 93), AARFAAK (SEQ ID No. 94), AArFAAK (SEQ ID No. 95), AAQFAAK (SEQ ID No. 96), AAqFAAK (SEQ ID No. 97), AAEFAAK (SEQ ID No. 98), AAeFAAK (SEQ ID No. 99), AADFAAK (SEQ ID No. 100), AAdFAAK (SEQ ID No. 101), AAPFAAK (SEQ ID No. 102), AApFAAK (SEQ ID No. 103), AAGFAAK (SEQ ID No. 104), AAgFAAK (SEQ ID No. 105), wherein the first A is protected in each sequence by Abz, and the final K is protected by DNP-OH, respectively. Furthermore, FAAK(DNP)-OH (SEQ ID No. 155) is obtained as product.

TABLE 5

| | A_{s, app}(SEQ ID | A_{s, app}(SEQ ID | A_{s, app}(SEQ ID | A_{s, app}(SEQ ID | A_{s, app}(SEQ ID |
|---|---|---|---|---|---|
| Xaa | No. 4) | No. 6) | No. 7) | No. 8) | No. 9) |
| Phe | n.d. \| 7.7 | <10⁻⁴ \| <10⁻³ | <10⁻⁵ \| 2.6 | <10⁻³ \| 0.80 | <10⁻³ \| 34.2 |
| Trp | n.d. \| 3.9 | n.d. \| <10⁻³ | n.d. \| 0.70 | <10⁻⁴ \| 0.011 | <10⁻⁴ \| 14.7 |
| Tyr | n.d. \| 2.2 | <10⁻⁴ \| <10⁻³ | <10⁻⁵ \| 0.64 | <10⁻⁴ \| 0.050 | <10⁻³ \| 29.5 |
| Ala | <10⁻⁴ \| 0.070 | <10⁻⁵ \| <10⁻⁵ | n.d. \| <10⁻³ | <10⁻³ \| 0.001 | <10⁻⁴ \| <10⁻³ |
| Val | <10⁻³ \| 0.090 | <10⁻⁴ \| n.d. | <10⁻⁵ \| <10⁻³ | <10⁻³ \| <10⁻³ | <10⁻⁴ \| <10⁻³ |
| Leu | <10⁻⁴ \| 2.7 | <10⁻⁴ \| <10⁻⁴ | <10⁻⁵ \| 0.021 | <10⁻³ \| 0.008 | <10⁻³ \| 0.013 |
| Ser | <10⁻⁴ \| 0.004 | <10⁻⁵ \| <10⁻⁵ | <10⁻⁵ \| <10⁻⁴ | <10⁻³ \| <10⁻³ | <10⁻⁴ \| <10⁻³ |
| Met | <10⁻⁴ \| 1.1 | <10⁻⁴ \| <10⁻⁴ | <10⁻⁴ \| 0.007 | <10⁻³ \| 0.021 | <10⁻³ \| 0.033 |
| His | n.d. \| 7.7 | <10⁻⁴ \| <10⁻⁴ | <10⁻⁴ \| <10⁻³ | <10⁻³ \| 0.005 | <10⁻⁴ \| 0.077 |
| Lys | <10⁻⁴ \| <10⁻³ | <10⁻⁴ \| 0.014 | n.d. \| n.d. | <10⁻⁴ \| <10⁻³ | <10⁻⁵ \| <10⁻⁵ |
| Arg | <10⁻⁴ \| n.d. | <10⁻⁴ \| 0.002 | n.d. \| n.d. | <10⁻³ \| <10⁻³ | <10⁻⁴ \| <10⁻⁵ |
| Gln | <10⁻⁵ \| 0.013 | <10⁻⁵ \| <10⁻⁴ | <10⁻⁵ \| <10⁻⁴ | <10⁻³ \| <10⁻⁴ | <10⁻⁴ \| <10⁻³ |
| Glu | n.d. \| 7.7 | <10⁻⁴ \| n.d. | n.d. \| n.d. | <10⁻³ \| <10⁻⁴ | <10⁻⁴ \| <10⁻⁴ |
| Asp | <10⁻³ \| <10⁻³ | <10⁻⁴ \| <10⁻⁵ | <10⁻⁴ \| <10⁻⁴ | <10⁻³ \| <10⁻⁴ | <10⁻³ \| <10⁻³ |
| Pro | n.d. \| 7.7 | <10⁻⁵ \| n.d. | n.d. \| n.d. | <10⁻⁴ \| <10⁻⁴ | <10⁻⁴ \| n.d. |
| Gyl | <10⁻⁴ | n.d. | <10⁻⁵ | <10⁻³ | <10⁻⁴ |

n.d. = not detectable

As can be seen from Table 5, the D-stereospecific hydrolytic enzymes are more active using a D-amino acid-containing substrate compared to the L-amino acid-containing substrate, similarly to the above.

In Table 6, the respective catalytic parameters of the hydrolysis reaction using D-stereospecific hydrolytic enzymes with SEQ ID No. 8 and No. 9 with quenched fluorogenic substrates (Abz-AAX/xₐₐ FAAK(DNP)-OH) are shown. The sequences in Table 6 are as follows: AAfFAAK (SEQ ID No. 68), AAwFAAK (SEQ ID No. 69), AAyFAAK(SEQ ID No. 70), AAlFAAK (SEQ ID No. 71), AAmFAAK (SEQ ID No. 72), AAhFAAK (SEQ ID No. 73) wherein the first A is protected in each sequence by Abz, and the final K is protected by DNP-OH, respectively.

TABLE 6

| | SEQ ID No. 8 | | | SEQ ID No. 9 | | |
|---|---|---|---|---|---|---|
| | K_{M} | k_{cat} | k_{cat}/K_{M} | K_{M} | k_{cat} | k_{cat}/K_{M} |
| xₐₐ | [μM] | [s⁻¹] | [M⁻¹ · s⁻¹] | [μM] | [s⁻¹] | [M⁻¹ · s⁻¹] |
| D-Phe | 6.8 | 0.68 | 9.9 · 10⁴ | 31.9 | 47.9 | 1.5 · 10⁶ |
| D-Trp | 5.0 | 0.08 | 1.6 · 10⁴ | 18.0 | 29.2 | 1.6 · 10⁶ |
| D-Tyr | 5.6 | 0.06 | 1.2 · 10⁴ | 22.9 | 34.2 | 1.5 · 10⁶ |
| D-Leu | n.d. | n.d. | n.d. | 57.8 | 0.01 | 1.9 · 10² |
| D-Met | 8.6 | 0.02 | 2.3 · 10³ | 66.7 | 0.03 | 4.8 · 10² |
| D-His | n.d. | n.d. | n.d. | 74.0 | 0.11 | 1.4 · 10³ |

Similar experiments were carried out using the D-stereospecific hydrolytic enzyme with SEQ ID No. 3 and the substrate Abz-AAX/xₐₐFAAK(DNP)-OH. The specific activity (As) is shown in Table 7.

TABLE 7

| Amino acid | A_{S} [mkat · mol⁻¹] | Amino acid | A_{S} [mkat · mol⁻¹] |
|---|---|---|---|
| L-Phe | 0.31 | D-Phe | 2.63 |
| L-Trp | n.d. | D-Trp | 1.93 |
| L-Tyr | 0.18 | D-Tyr | 0.63 |
| L-Met | 0.32 | D-Met | 0.15 |
| L-His | 0.21 | D-His | n.d. |
| L-Ser | 0.28 | D-Ser | n.d. |
| L-Gln | 0.24 | D-Gln | n.d. |
| L-Glu | n.d. | D-Glu | n.d. |
| L-Arg | 0.24 | D-Arg | n.d. |
| L-Pro | n.d. | D-Pro | n.d. |
| L-Lys | 0.30 | D-Lys | n.d. |
| L-Ala | 0.39 | D-Ala | n.d. |
| L-Leu | 0.20 | D-Leu | 0.61 |
| L-Val | 0.20 | D-Val | n.d. |
| L-Asp | n.d. | D-Asp | 1.98 |
| Gly | n.d. | — | — |

### Experiments Regarding the S₂-Preference of D-Stereospecific Hydrolytic Enzymes

In further experiments, the S₂-preference of D-stereospecific hydrolytic enzymes using D-stereospecific hydrolytic enzymes with SEQ ID No. 8 and No. 9 was investigated using the substrate Abz-AX/xₐₐfAAAK(DNP)-OH, as above with the IQFS. The respective model reaction is shown below (Formula 6), wherein AAAK(DNP)-OH (SEQ ID No. 156) is obtained as product. In Formula 6 DHy represents again the D-stereospecific hydrolytic enzyme.

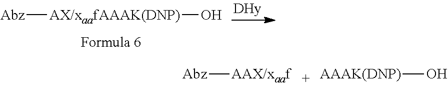

The following SEQ ID apply: Abz-AXfAAAK(DNP)-OH (SEQ ID No. 174), Abz-AxfAAAK(DNP)-OH (SEQ ID No. 175), AAAK(DNP)-OH (SEQ ID No. 156).

In Table 8, the specific activity Aₛ (kat/mol) of the D-stereospecific enzymes are shown. In Table 8, the amino acid in P2 position is printed in bold and the cleavage site is marked with an arrow.

TABLE 8

| | Specific activity | |
|---|---|---|
| | [kat/mol] | |
| Substrate | SEQ ID No. 8 | SEQ ID No. 9 |
| Abz-AMf↓AAA-K(DNP)-OH (SEQ ID No. 39) | 10.34 ± 0.11 | 11.40 ± 0.24 |
| Abz-AFf↓AAA-K(DNP)-OH (SEQ ID No. 40) | 7.36 ± 0.20 | 42.00 ± 0.55 |
| Abz-AYf↓AAA-K(DNP)-OH ((SEQ ID No. 41) | 5.47 ± 0.27 | 33.47 ± 0.75 |
| Abz-AWf↓AAA-K(DNP)-OH (SEQ ID No. 42) | 0.38 ± 0.04 | 3.03 ± 0.17 |
| Abz-AVf↓AAA-K(DNP)-OH (SEQ ID No. 43) | 2.25 ± 0.20 | 13.99 ± 0.45 |
| Abz-ALf↓AAA-K(DNP)-OH (SEQ ID No. 44) | 5.97 ± 0.17 | 8.19 ± 0.40 |
| Abz-AIf↓AAA-K(DNP)-OH (SEQ ID No. 45) | 4.30 ± 0.29 | 11.43 ± 0.16 |
| Abz-AAf↓AAA-K(DNP)-OH (SEQ ID No. 46) | 2.73 ± 0.38 | 16.61 ± 1.14 |
| Abz-AGf↓AAA-K(DNP)-OH (SEQ ID No. 47) | 1.02 ± 0.06 | 3.55 ± 0.18 |
| Abz-APf↓AAA-K(DNP)-OH (SEQ ID No. 48) | 1.60 ± 0.08 | 1.23 ± 0.06 |
| Abz-ASf↓AAA-K(DNP)-OH (SEQ ID No. 49) | 0.65 ± 0.02 | 4.97 ± 0.19 |
| Abz-AEf↓AAA-K(DNP)-OH (SEQ ID No. 50) | 0.04 ± 0.00 | 0.20 ± 0.00 |
| Abz-ADf↓AAA-K(DNP)-OH (SEQ ID No. 51) | 0.03 ± 0.00 | 0.06 ± 0.00 |
| Abz-AQf↓AAA-K(DNP)-OH (SEQ ID No. 52) | 2.30 ± 0.14 | 1.69 ± 0.05 |
| Abz-AKf↓AAA-K(DNP)-OH (SEQ ID No. 53) | 0.26 ± 0.01 | 0.57 ± 0.01 |
| Abz-ARf↓AAA-K(DNP)-OH (SEQ ID No. 54) | 0.78 ± 0.01 | 1.42 ± 0.03 |
| Abz-AHf↓AAA-K(DNP)-OH (SEQ ID No. 55) | 0.97 ± 0.01 | 4.65 ± 0.04 |

In Table 9, the specific activities of the D-stereospecific enzymes and the kinetic parameters are shown.

TABLE 9

| | SEQ ID No. 3 | | | | SEQ ID No. 4 | | | |
|---|---|---|---|---|---|---|---|---|
| c | Vₘₐₓ | k_{cat} | K_{M} | k_{cat}/K_{M} | Vₘₐₓ | k_{cat} | K_{M} | k_{cat}/K_{M} |
| X | [nmol/s] | [s⁻¹] | [μM] | [M⁻¹ · s⁻¹] | [nmol/s] | [s⁻¹] | [μM] | [M⁻¹ · s⁻¹] |
| M | 182.8 ± 0.50 | 17.0 ± 0.0 | 49.1 ± 1.30 | 3.5 × 10⁵ | *16.5 ± 2.3 | 14.5 ± 0.2 | 13.2 ± 3.8 | 1.1 × 10⁶ |
| F | 299.2 ± 13.5 | 14.9 ± 0.7 | 92.9 ± 14.2 | 1.6 × 10⁵ | 53.1 ± 1.0 | 46.1 ± 0.9 | 18.0 ± 1.8 | 2.6 × 10⁶ |
| Y | 87.4 ± 3.00 | 8.2 ± 0.3 | 58.6 ± 1.90 | 1.4 × 10⁵ | 61.2 ± 0.4 | 53.7 ± 0.3 | 35.0 ± 0.8 | 1.5 × 10⁶ |
| V | — | — | — | — | 30.5 ± 2.0 | 58.0 ± 3.7 | 26.5 ± 1.8 | 4.6 × 10⁵ |
| I | — | — | — | — | 23.8 ± 1.6 | 20.7 ± 1.6 | 52.4 ± 8.9 | 4.0 × 10⁵ |

As can be seen from Tables 8 and 9, the D-stereospecific enzymes recognize specifically the D-amino acid or the D-amino acid derivative when the D-amino acid or D-amino acid is in P2 position, which underlines the broad variability for the application of the D-stereospecific enzymes.

### Experiments Regarding the S₁′-Preference of D-Stereospecific Hydrolytic Enzymes

In further experiments, the S₁′-preference of D-stereospecific hydrolytic enzymes using D-stereospecific hydrolytic enzymes with SEQ ID No. 8 and No. 9 was investigated using the substrate Abz-AAfxₐₐAAK(DNP)-OH.

In Table 10, the specific activity Aₛ (kat/mol) of the D-stereospecific enzymes are shown. In Table 10, the amino acid in P1′ position is printed in bold and the cleavage site is marked with an arrow.

TABLE 10

| | Specific activity | |
|---|---|---|
| | [kat/mol] | |
| Substrate | SEQ ID No. 8 | SEQ ID No. 9 |
| Abz-AAf↓MAA-K(DNP)-OH (SEQ ID No. 56) | 1.97 ± 0.05 | 42.10 ± 0.72 |
| Abz-AAf↓FAA-K(DNP)-OH (SEQ ID No. 57) | 0.41 ± 0.00 | 68.30 ± 1.28 |
| Abz-AAf↓VAA-K(DNP)-OH (SEQ ID No. 58) | 0.09 ± 0.02 | 14.10 ± 0.34 |
| Abz-AAf↓LAA-K(DNP)-OH (SEQ ID No. 59) | 0.45 ± 0.02 | 21.60 ± 0.07 |
| Abz-AAf↓AAA-K(DNP)-OH (SEQ ID No. 60) | 0.28 ± 0.00 | 8.40 ± 0.06 |
| Abz-AAf↓GAA-K(DNP)-OH (SEQ ID No. 61) | 0.19 ± 0.01 | 13.10 ± 0.16 |
| Abz-AAf↓PAA-K(DNP)-OH (SEQ ID No. 62) | n.d. | n.d. |
| Abz-AAf↓SAA-K(DNP)-OH (SEQ ID No. 63) | 0.28 ± 0.01 | 8.70 ± 0.60 |
| Abz-AAf↓NAA-K(DNP -OH (SEQ ID No. 64) | 0.06 ± 0.02 | 10.50 ± 0.07 |
| Abz-AAf↓EAA-K(DNP)-OH (SEQ ID No. 65) | 0.00 ± 0.02 | 0.10 ± 0.05 |
| Abz-AAf↓RAA-K(DNP)-OH (SEQ ID No. 66) | 0.14 ± 0.01 | 3.20 ± 0.08 |
| Abz-AAf↓AA-K(DNP)-OH (SEQ ID No. 67) | 0.25 ± 0.01 | 37.80 ± 0.18 |

The following products are obtained after cleavage, apart from Abz-AAf: MAAK (SEQ ID No. 157), FAAK (SEQ ID No. 158), VAAK (SEQ ID No. 159), LAAK (SEQ ID No. 160), AAAK (SEQ ID No. 161), GAAK (SEQ ID No. 162), PAAK (SEQ ID No. 163), SAAK (SEQ ID No. 164), NAAK (SEQ ID No. 165), EAAK (SEQ ID No. 166), RAAK (SEQ ID No. 167), HAAK (SEQ ID No. 168).

From the experiments regarding the subsite mapping, the S₂-preference and the S₁′-preference, it can be seen that the enzymes are active regardless at which position the D-amino acid or D-amino acid derivative is located. Thus, the D-stereospecific-hydrolytic enzymes can be variably used with substrates having at least one D-amino acid or D-amino acid derivative protected functional group.

### Experiments to the Influence of Different Additives and Different Reaction Conditions on the Activity of D-Stereospecific Hydrolytic Enzymes

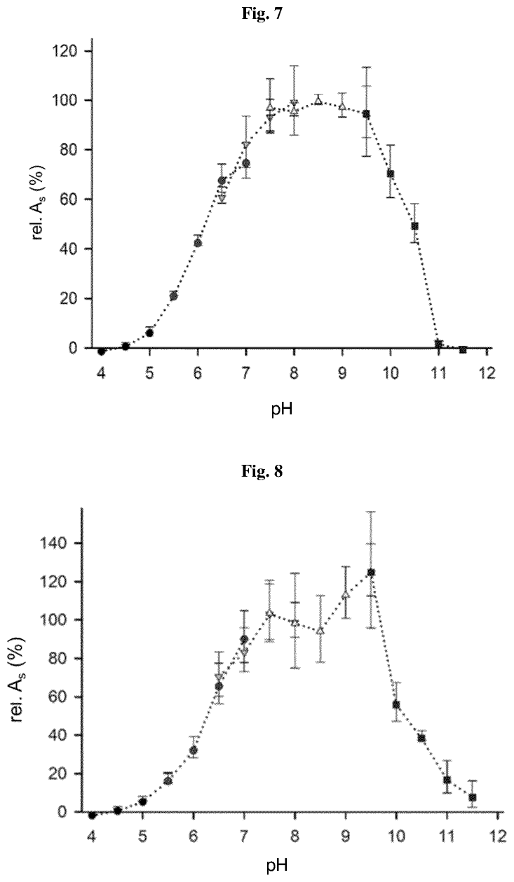

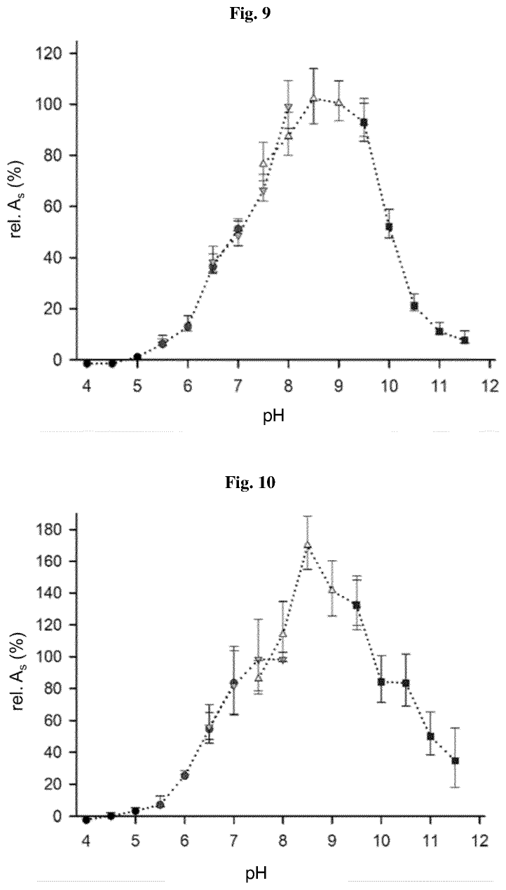

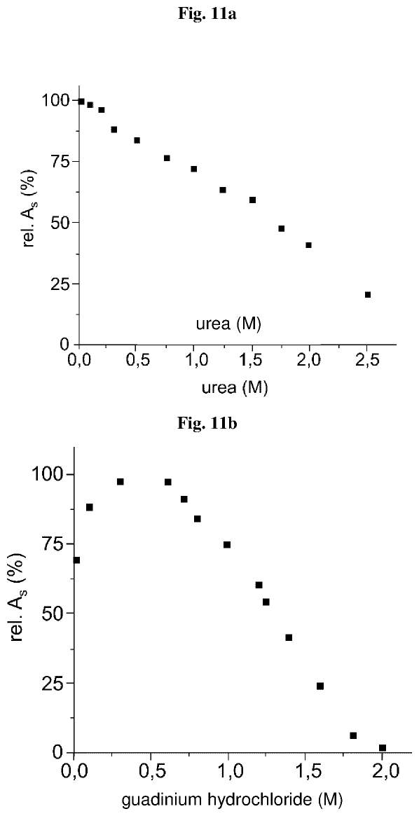

In further experiments, the effect of the pH value on the activity of D-stereospecific hydrolytic enzymes SEQ ID No. 4, No. 6, No. 7, No. 8 and No. 9 was investigated. As a substrate the peptide Abz-AAfFAAK(DNP)-OH was used. The pH value was varied by using different buffers (acetate buffer: pH 4.0-5.5; MES buffer; pH 5.5-7.0; NaPi buffer: pH 6.5-8.0; TAPS buffer: pH 7.5-9.5; CAPS buffer: pH 9.5-11.5). The relative activities (rel. Aₛ) of the respective enzymes are shown in FIGS. 6 to 10 (FIG. 6: SEQ ID No. 9; FIG. 7: SEQ ID No. 8; FIG. 8: SEQ ID No. 7; FIG. 9: SEQ ID No. 6; FIG. 10: SEQ ID No. 4). The relative activity is calculated by dividing the activity at a different pH value by the corresponding activity at pH 8.0 (which was used as standard condition) of the respective D-stereospecific enzyme.

As can be seen from FIG. 6 to 10, the D-stereospecific hydrolytic enzymes are active in a wide pH range. Therefore, the D-stereospecific hydrolytic enzymes can variably be used in different synthesis conditions.

In addition, experiments were performed to investigate the effect of adding different additives in different concentrations based on the reaction mixture on the activity of D-stereospecific hydrolytic enzymes. The concentration of the respective additive was varied between 0 to 2.5 M. As additives, urea, guanidinium hydrochloride, NaCl and ß-mercaptoethanol (ß-ME) were used. The additives were added to the reaction mixture including the D-stereospecific hydrolytic enzyme and the peptide Abz-AAfFAAK(DNP)-OH. As D-stereospecific hydrolytic enzymes the D-stereospecific hydrolytic enzymes with SEQ ID No. 4, No. 6, No. 7, No. 8 and No. 9 were used.

In FIGS. 11a and 11b the relative activity of the D-stereospecific hydrolytic enzyme with SEQ ID No. 9 is shown when adding the additives urea or guanidinium hydrochloride.

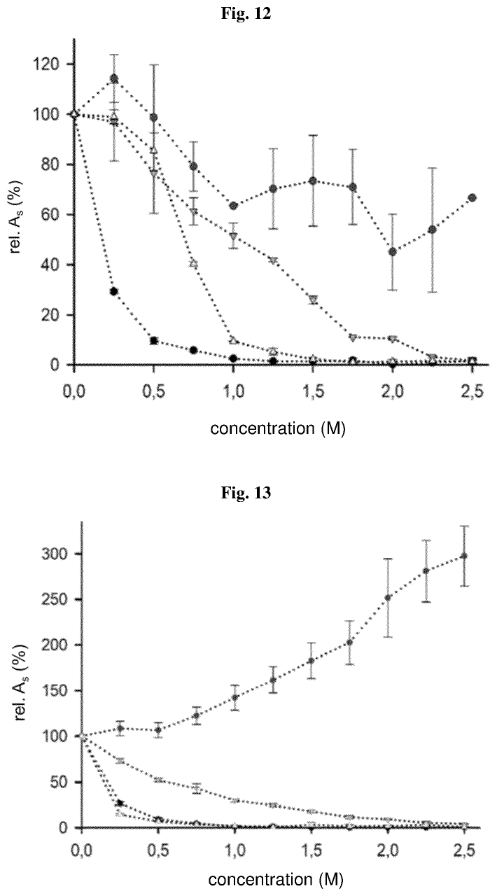

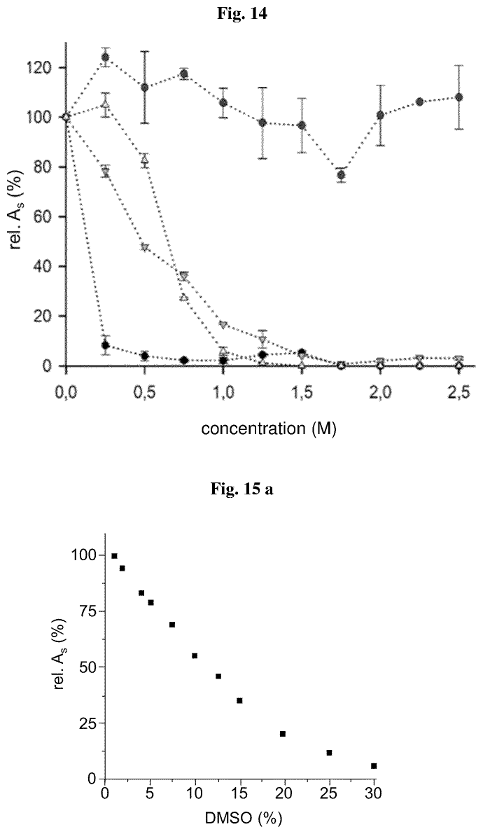

In FIG. 12 to 14 the relative activities of the D-stereospecific hydrolytic enzymes with SEQ ID No. 8 (FIG. 12), No. 6 (FIG. 13) and No. 4 (FIG. 14) are shown, when adding the additives urea, guanidinium hydrochloride, NaCl or ß-mercaptoethanol at different concentrations.

The relative activity, in this case, is calculated by dividing the activity when adding an additive with a different concentration by the maximum activity of the respective D-stereospecific enzyme.

In Table 11, the concentration of additive (M) based on the reaction mixture at which 50% (*) or 10% (**) of the relative specific activity of the respective enzyme is still present is indicated.

TABLE 11

| | β-ME (M) | | urea (M) | | GdmCl (M) | |
|---|---|---|---|---|---|---|
| enzyme | *50% | ** 10% | *50% | ** 10% | *50% | ** 10% |
| SEQ ID No. 6 | <0.25 | 0.50 | 0.50 | 2.00 | <0.25 | 0.50 |
| SEQ ID No. 7 | <0.25 | 0.50 | 1.00 | 1.50 | <0.25 | 0.50 |
| SEQ ID No. 8 | <0.25 | 0.50 | 1.00 | 2.00 | 0.75 | 1.00 |
| SEQ ID No. 9 | <0.25 | 0.50 | 0.75 | 2.25 | 1.00 | 1.25 |
| SEQ ID No. 4 | <0.25 | <0.25 | 0.50 | 1.25 | 0.75 | 1.00 |

As can be seen from Table 11, the D-stereospecific hydrolytic enzymes are active even if different additives are added to the reaction mixture. Therefore, the D-stereospecific hydrolytic enzymes can variably be used in different synthesis conditions.

Furthermore, experiments were carried out to investigate the influence of the solvent on the activity of D-stereospecific hydrolytic enzymes.

As solvents, DMSO, DMF, methanol and ACN (acetonitrile) were used. The concentration of the solvents was varied between 0 and 20% v/v (DMF, acetnotrile), 0 and 30% v/v (DMSO, methanol). The solvent was added to the reaction mixture including the D-stereospecific hydrolytic enzyme and the peptide Abz-AAfFAAK(DNP)-OH in different concentrations based on the volume of the entire solvent. The residual solvent was water. As D-stereospecific hydrolytic enzymes the D-stereospecific hydrolytic enzymes with SEQ ID No. 4, No. 6, No. 7, No. 8 and No. 9 were used.

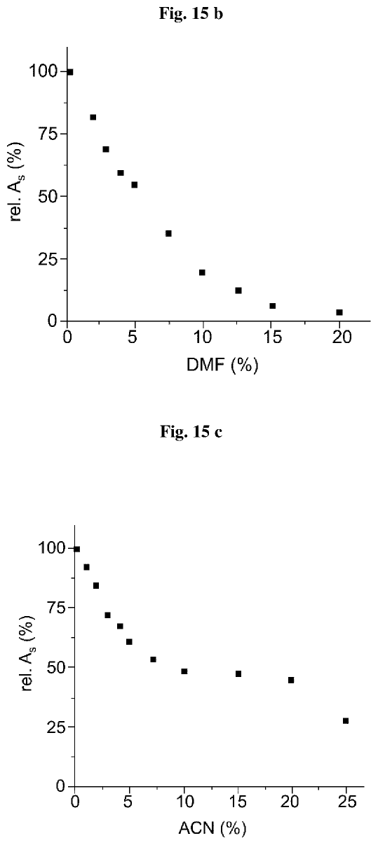

In FIG. 15a to 15c, the relative activity (rel. Aₛ) of the D-stereospecific hydrolytic enzyme with SEQ ID No. 9 is shown when DMSO (15a), ACN (15b) and DMF (15c) is added.

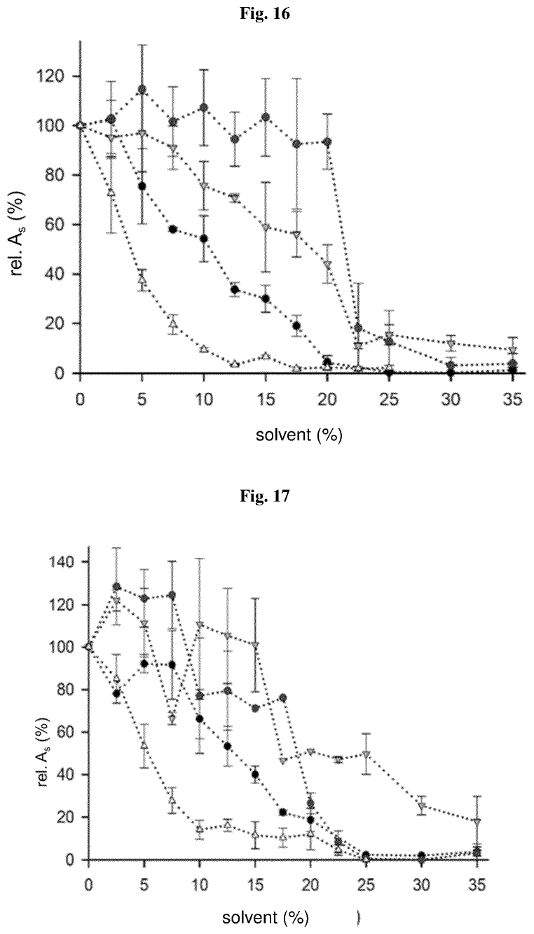

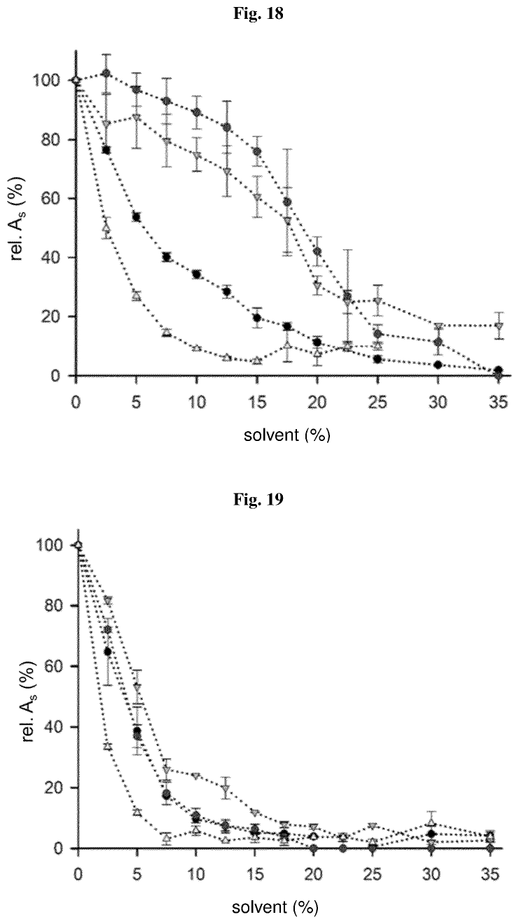

In FIG. 16 to 19 the relative activity (rel. Aₛ) of the D-stereospecific hydrolytic enzyme with SEQ ID No. 4 (FIG. 19), No. 6 (FIG. 18), No. 7 (FIG. 17) and No. 8 (FIG. 16) is shown when DMSO, ACN, methanol and DMF is added.

The relative activity, in this case, is calculated by dividing the activity when adding the solvent with a different concentration by the maximum activity of the respective D-stereospecific enzyme.

In Table 12, the influence of selected organic solvents on the activity of the D-stereospecific hydrolases is shown. The concentration of organic solvent (v/v) at which 50% (*) or 10% (**) of the relative specific activity of the respective enzyme is still present is indicated.

TABLE 12

| | DMSO (v/v) | | ACN (v/v) | | MeOH (v/v) | | DMF (v/v) | |
|---|---|---|---|---|---|---|---|---|
| enzyme | *50% | ** 10% | *50% | ** 10% | *50% | ** 10% | *50% | ** 10% |
| SEQ ID No. 6 | 5.0% | 20.0% | 20.0% | 30.0% | 17.5% | 35.0% | 2.5% | 10.0% |
| SEQ ID No. 7 | 12.5% | 20.0% | 20.0% | 22.5% | 17.5% | 35.0% | 5.0% | 20.0% |
| SEQ ID No. 8 | 10.0% | 20.0% | 22.5% | 30.0% | 17.5% | 30.0% | 5.0% | 10.0% |
| SEQ ID No. 9 | 10.0% | 20.0% | 20.0% | 22.5% | 17.5% | 22.5% | 2.5% | 10.0% |
| SEQ ID No. 4 | 5.0% | 10.0% | 5.0% | 10.0% | 5.0% | 15.0% | <2.5% | 5.0% |

As can be seen from FIG. 15 a to 15 c, 16 to 19 and Table 11, the D-stereospecific hydrolytic enzymes are active even a solvent other than water is added to the reaction mixture. Even if the concentration of the other solvent is 35% (v/v) the D-stereospecific hydrolytic enzymes are active. Therefore, the D-stereospecific hydrolytic enzymes can variably be used in different synthesis conditions.

### Alignment of Isolated and Used D-Stereospecific Hydrolytic Enzymes

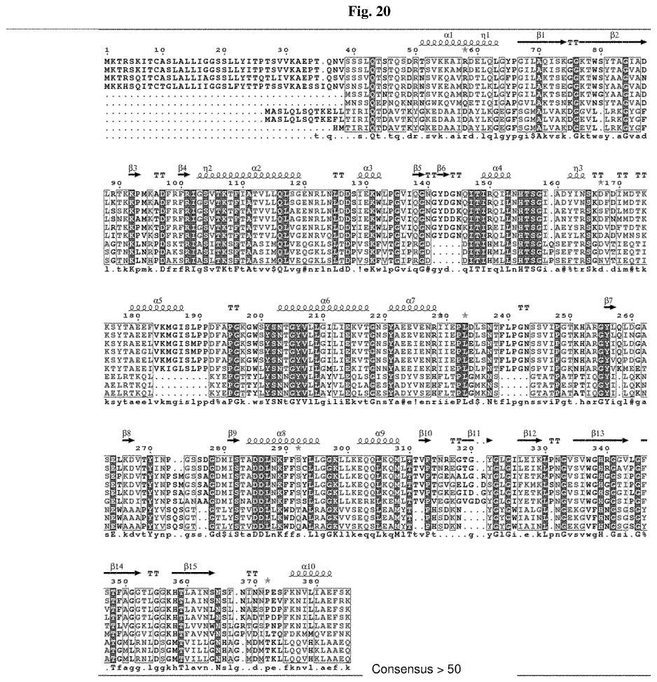

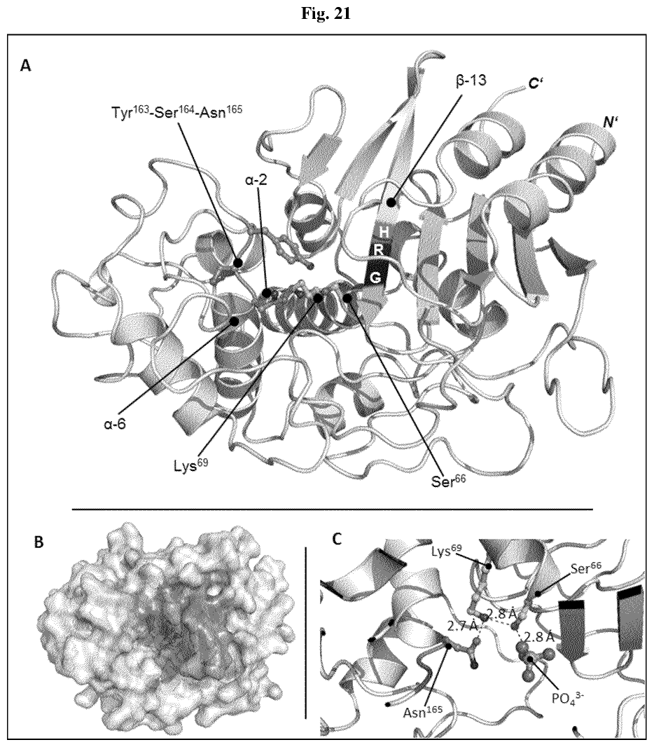

In FIG. 20, the alignment of isolated and used D-stereospecific hydrolytic enzymes is shown. In the alignment, conserved amino acids are highlighted in red, amino acids with similar functions are written in red, and conserved regions are framed. The numbering is based on the complete numbering of the consensus sequence. In addition, secondary structure elements (α-α-helix, η-3₁₀-helix, β-β-sheet, TT-β-turn) are labeled according to SEQ ID No. 6, for which a crystal structure was obtained with a resolution of 1.46 Å and which is shown in FIG. 21. For obtaining crystals, SEQ ID No. 6. was concentrated to 120 μM in 0.1 M phosphate buffer (pH 8.0) and 0.1 M NaCl and crystallized by hanging drop vapor diffusion at 20° C. by mixing with an equal volume of the precipitant solution 0.1 M imidazole, 0.1 M MES pH 6.5, 30 mM MgCl₂, 30 mM CaCl₂), 12.5% (v/v) MPD, 12.5% (w/v) polyethylene glycol 1000 and 12.5% (w/v) polyethylene glycol 3350. Crystals appeared within 14 to 21 days and were flash-frozen without any additional cryo-protectant. Data collection was carried out at −172° C. at the BESSY synchrotron beamlines 14.1 and 14.2 (Helmholtz Zentrum, Berlin). Crystals belong to the monoclinic space group P2₁. Shown is the structure (A) with the conserved motives SXXK und YXN. Additionally shown are the HRG-motive, which is in β-sheet 13, and α-helices 2 und 6. Also shown are the surface and the assumed bonding site and a co-crystallized phosphate in detail (B), as well as the orientation of the phosphate in the active center and the distances to the catalytically active important side chains (C). Additional crystal structure results of soaking experiments with the substrate Bz-D-Arg-OMe confirmed the bonding to the substrate and the mechanism (data not shown).

Alignment was performed using MultAlign and visualization was performed using ESPript3 (Corpet, 1988; Robert & Gouet, 2014)

As can be seen from FIG. 20, all sequences of the isolated enzymes have a 3₁₀-helix 2 structure including an amino sequence SXXK, an α-helix structure (α-helix-6 structure) including an amino acid sequence YSN, and a β-sheet structure including the amino acid sequence HXG ((β-sheet-13).

The use of D-amino acids and/or D-amino acid derivatives, e.g. as protecting groups or linkers for affinity probes or solubility tags, is compatible with standard synthesis protocols in peptide and carbohydrate chemistry, and thus can be easily applied to already established synthesis protocols without additional effort. In contrast to e.g. penicillin acylases, the biocatalysts used here are highly effective for the reactions considered, e.g. with specificity constants of up to 7*10⁶ M⁻¹ s⁻¹. This allows quantitative conversions at low enzyme concentrations in very short reaction times. The enzymes are expressible in high yields using standard molecular biology and protein chemistry protocols and are tolerant to a wide range of additives and organic solvents. Moreover, the biocatalysts used do not require cofactors or stabilizers and are stable and catalytically active over a wide pH range.

Experiments with Different Substrates Using D-Stereospecific Hydrolytic Enzymes

In order to show that the present D-stereopsecific hydroylytic enzymes are also useful for non-proteinogenic substrates, experiments were conducted with different substrates, similar to the above, with the following conditions: 100 μM substrate, 10-50 nM enzyme, 100 mM NaPi pH=8, 150 mM NaCl, 30° C. The evaluation was UPLC-based at 254 nM. Ahx-NH₂=aminohexanoic acid amide, Glc=glucosamine. The results are shown in Table 13.

TABLE 13

A_{S} (mkat/mol)

| | SEQ ID | SEQ ID | SEQ ID | SEQ ID |
|---|---|---|---|---|
| substrate | No. 9 | No. 7 | No. 8 | No. 4 |
| Bz-f-Ahx-NH₂ | 15.200 | 3.100 | 190 | 1.500 |
| Fmoc-f-Glc | 4.800 | 480 | 190 | 660 |

With regard to the sequence listing it is noted that ID No. 1 to 9 in the sequence listing correspond to SEQ ID No. 1 to 9, ID No. 10 to 27 in the sequence listing correspond to SEQ ID No. 1A, 1B, 2A, 2B, 3A, 3B, 4A, 4B, 5A, 5B, 6A, 6B, 7A, 7B, 8A, 8B, 9A, 9B, and ID No. 28 to 193 in the sequence listing correspond to SEQ ID No. 10 to 175.

1. A method of deprotecting at least one D-amino acid and/or D-amino acid derivative protected functional group of at least one substrate, the at least one substrate having at least one D-amino acid and/or D-amino acid derivative protected functional group, using a D-stereospecific hydrolytic enzyme, the method comprising:
Providing the at least one substrate, the D-stereospecific hydrolytic enzyme, and at least one solvent;
Mixing the at least one substrate p, the D-stereospecific hydrolytic enzyme and the at least one solvent to obtain a reaction mixture; and
Hydrolyzing the at least one D-amino acid and/or D-amino acid derivative protected functional group of the at least one substrate using the D-stereospecific hydrolytic enzyme in the reaction mixture to obtain a D-amino acid and/or a D-amino acid derivative and an at least partially deprotected substrate with at least one deprotected functional group deprotected from the D-amino acid and/or D-amino acid derivative.

2. The method according to claim 1, wherein the at least one substrate is selected from the group consisting of a carbohydrate, a carbohydrate derivative, a nucleotide, an oligonucleotide, a polynucleotide, a nucleotide derivative, an oligonucleotide derivative, a polynucleotide derivative, an amino acid, a peptide, an oligopeptide, a polypeptide, a protein, a peptide intermediate, a peptide derivative, an oligopeptide derivative, a polypeptide derivative, and mixtures thereof; particularly wherein the substrate has at least two functional groups.

3. The method according to claim 1, further comprising adding a buffer to the reaction mixture, wherein the pH value of the reaction mixture is in the range from 3 to 13.

4. The method according to claim 1, wherein the functional group is selected from the group consisting of an amino group, a carboxyl group, a hydroxyl group, a sulfhydryl group, and a selenohydryl group.

5. The method according to claim 4, wherein the functional group is an amino group, a hydroxyl group, a sulfhydryl group and/or a selenohydryl group and the D-amino acid and/or D-amino acid derivative binds via the carbonyl group to the substrate.

6. The method according to claim 1, wherein the concentration of the substrate in the reaction mixture is in the range from 100 nM to 1 M based on the reaction mixture.

7. The method according to claim 1, wherein the concentration of the D-stereospecific hydrolytic enzyme in the reaction mixture is in the range from 1 nM to 200 μM based on the reaction mixture.

8. The method according to claim 1, wherein the at least one solvent comprises at least water.

9. The method according to claim 3, wherein the buffer is selected from the group consisting of acetate buffer, 2-(N-morpholino)ethanesulfonic acid (MES) buffer, sodium phosphate (NaPi) buffer, potassium phosphate (KPi) buffer, 2-(4-(2-hydroxyethyl)-piperazin-1-yl)-ethane-1-sulfonic acid (HEPES) buffer, tris-(hydroxymethyl)-aminomethane (Tris) buffer, piperazine-N,N′-bis(2-ethanesulfonic acid) (PIPES) buffer, bis-(2-hydroxyethyl)-imino-tris-(hydroxymethyl)-methane (Bis-Tris) buffer, 1,3-bis-(tris-(hydroxymethyl)-methylamino)-propane (Bis-Tris propane) buffer, N-(2-acetamido)iminodiacetic acid (ADA) buffer, N-(2-acetamido)-2-aminoethanesulfonic acid (ACES) buffer, acetamido glycine buffer, 2-hydroxy-3-morpholinopropanesulfonic acid (MOPSO) buffer, N,N-bis-(2-hydroxyethyl)-2-amino-ethansulfonic acid (BES) buffer, 3-(N-morpholino)-propanesulfonic acid (MOPS) buffer, 2-((1,3-dihydroxy-2-(hydroxymethyl)-propan-2-yl)-amino)ethane-1-sulfonic acid (TES) buffer, cholamine chloride hydrochloride, 4-(N-morpholino)-butanesulfonic acid (MOBS) buffer, 3-N-bis-(hydroxyethyl)-amino-2-hydroxy-propansulfonic acid (DIPSO) buffer, 3-((1,3-dihydroxy-2-(hydroxymethyl)-propan-2-yl)-amino)-2-hydroxypropane-1-sulfonic acid (TAPSO) buffer, triethanolamine buffer, 4-(2-hydroxyethyl)-piperaziri-1-(2-hydroxy-propansulfonic acid) (HEPPSO) buffer, piperazin-N,N′-bis-(2-hydroxypropansulfonic acid) (POPSO) buffer, N-(tri(hydroxymethyl)-methyl)-glycine (Tricine) buffer, glycylglycine buffer, gylcinamide buffer, (bis(2-hydroxyethyl)-amino)acetic acid (BICINE) buffer, N-tris(hydroxymethyl)-methyl-3-aminopropanesulfonic acid (TAPS) buffer, 2-amino-2-methyl-1,3-propanediol (AMPD) buffer, N-tris(hydroxymethyl)-methyl-4-aminobutanesulfonic acid (TABS) buffer, N-(1,1-dimethyl-2-hydroxyethyl)-3-amino-2-hydroxypropansulfonic acid (AMPSO) buffer, N-cyclohexyl-2-aminoethanesulfonic acid (CHES) buffer, 3-(cyclohexylamino)-2-hydroxy-1-propansulfonic acid (CAPSO) buffer, (2-amino-2-methylpropan-1-ol) (AMP) buffer and 3-(cyclohexylamino)propane-1-sulfonic acid (CAPS) buffer.

10. The method according to claim 1, wherein the hydrolysis is carried out at a reaction temperature in the range from 0° C. to 50° C.

11. A D-stereospecific hydrolytic enzyme, characterized by the following features:
at least one first 3₁₀-helix-2 structure comprising an amino acid sequence SXXK;
at least one first α-helix structure comprising an amino acids sequence YSN; and
at least one first μ-sheet structure comprising an amino acid sequence HXG;
wherein X is an amino acid selected from the group consisting of proteinogenic amino acids;
wherein the first 3₁₀-helix-2 structure, the first α-helix structure, and the first β-sheet structure are arranged in close proximity.

12. The D-stereospecific hydrolytic enzyme according to claim 11, wherein the D-stereospecific hydrolytic enzyme has an amino acid sequence chosen from SEQ ID No. 1, No. 2 No. 3, No. 4, No. 5, No. 6, No. 7, No. 8 and No. 9:
SEQ ID No. 1:
ASLQLSQTKELLTIRIQTDAVTKYGKEDAAIDAYLKGEGFSGMALVAKDGGVLLRKGYGFAGT
NKLNRPDSKTRIASITKSFTAASIMQLVEQGKLSLTDPVSKFVTGIPRGDDITIHMLLSHTSG
LQSEFTRSGDVTIEQTIEELRTKQLKYEPGTTYLYSNNGYVLLAYVLEQLSGISYSDYVSEHF
LTPLGMKNSGTATPATPTIQGYILQKNNEWAAAPYYVSQSGTGTLYSTVDDLLKWDTALRAGK
VVSEQSLEAMYTPHSDKNYGYGWIALGLNGEKGVFHNGSGSGYATGMLRNLDSGMTVILLGNH
AGMDMTKLLQQVHKLAAEQ;
SEQ ID No. 2:
TIRIQTDAVTKYGKEDAAIDAYLKGEGFSGMALVAKDGEVLLRKGYGFSGTNKLNHPDAKSRI
ASLTKSFTAASIMQLVEQGKLSLTDPVSKFVTGIPRGDDITIHMLLSHTSGLPSEFTRSGNVT
IEQTIAELRTKQLKYEPGTTYLYSNNGYVLLAYVLEQLSGESYADYVSEHELTPLGMKNSGTA
TPATATIQGYILQKNNEWAAAPYYVSQSGTGTLYSTVDDLLKWDQALRAGKVVSKQSLEAMYT
PHSDKNYGYGWIAINLNGEKGVFHINGSGSGYATGMLRNLDSGMTVILLGNHAGMDMTKLLQQ
VHKLAAEQ;
SEQ ID No. 3:
ASLQLSQTKEFLTIRIQTDAVTKYGKEDAAIDAYLKGEGFSGMALVAKDGEVLLRKGYGFSGT
NKLNRPDAKSRIASITKSFTAASIMQLVEQGKLSLTDPVSKFVTGIPRGDDITIHMLLSHTSG
LPSEFTRSGDVTIEQTIAELRTKQLKYEPGTTYLYSNNGYVLLAYVLEQLAGESYADYVNEHE
LTPLGMKNSGTATPESPTIQGYILQKNNEWAAAPYYVSQSGTGTLYSTVDDLLKWDQALRAGK
VVSEQSLEAMYTPHSDKNYGYGWIAINLNGEKGVFHNGSGSGYATGMLRNLDSGMTVILLGNH
AGMDMTKLLQQVHKLAAEQ;
SEQ ID No. 4:
SSVQTSTQRDRNSVKQAVRDTLQLGFPGILAKTSEGGKTWSYAAGVANLSSKKPMKTDFRFRI
GSVTKTFTATVVLQLAEENRLNLDDSIEKWLPGVIQGNGYDDKQITIRQLLNHTSGIAEYTRS
KSFDLMDTKKSYRAEELVKMGISMPPDFAPGKSWSYSNTGYVLLGILIETVTGNSYAEEIENR
IIEPLELSNTFLPGNSSVIPGTKHARGYIQLDGASEPKDVTYYNPSMGSSAGDMISTADDLNK
FFSYLLGGKLLKEQQLKQMLTTVPTGEAALGRYGLGIYETKLPNGVSIWGHGGSIPGFVTFAG
GTLGGKHTLAVNLNSLNAESPDPFKNILLAEFSK;
SEQ ID No. 5:
SSLQTSTQSDRTSVKKAMRDELQLGYPGILAKISKGGKTWSYTAGVADLKTKKPMKADFRFRI
GSVTKTFIATVLLQLSGENRLNLDDSIEKWLPGVIQGNGYDGNQITIRQILNHTSGIADYVNS
KDFDIMDTKKSYTAEEFVKMGISLPPDFAPGKGWSYSNTGYVLLGILIEKVTGNSYAEEVENR
IIEPLDLSNTFLPGNSSVIPGTKHARGYLQINGASELKDVTYINPGSSDGDMISTADDLNKFF
SCLLGGKLLKEQQLKQMLTTVPTNREGTGYGLGILEIKLPNGVSVWGHRGAVPGFSTFAGGTL
GGKHTLAINSNSLNLNNPEVFKNILLAEFRK;
SEQ ID No. 6:
SSLQTSTQSDRTSVKKAIRDELQLGYPGILAQISKGGKTWSYTAGIADLRTKKPMKADFRFRI
GSVTKTFIATVLLQLSGENRLNLDDSIEKWLPGVIQGNGYDGNQITIRQILNHTSGIADYINS
KDFDIMDTKKSYTAEEFVKMGISLPPDFAPGKGWSYSNTGYVLLGILIEKVTGNSYAEEVENR
IIEPLDLSNTFLPGNSSVIPGTKHARGYLQLDGASELKDVTYINPGSSDGDMISTADDLNKFF
SYLLGGKLLKEQQLKQMLTTVPTNREGTGYGLGILEIKLPNGVSVWGHRGGVLGFSTFAGGTL
GGKHTLAINSNSFNINNPESFKNVLIAEFSK;
SEQ ID No. 7:
NSLQTSTQRDRNSVKEAMRDTLKLGYPGILAKTSEGGKTWSYAAGVADLSNKKAMKTDFRFRI
GSVTKTFTATVVLQLAGENRLNLDDSIEKWLPGVIQGNGYDDKQITIRQLLNHTSGIANYTRS
KDFNMMDTKKSYTAEGLVKMGISMPPDFAPGKSWSYSNTGYVLLGILIEKVTGNSYAEEIENR
IIEPLELANTFLPGNSSVIPGTKHARGYIQLDGASETKDVTYYNPSMGSSAGDMISTADDLNK
FFSYLLGGKLLKEQQLKQMLTTVPTGVGELGDSGLGIFKMKLSNGESIWGHGGTIPGFLTFAG
GTLGGKHTLAVNLNSLKADTPDPFKNILLAEFSK;
SEQ ID No. 8:
SSLQTNTQRDRTSVKQAMRDTLQLGYPGILAKTSEDGKTWGYAAGIADLRTKKPMKTDFRFRI
GSVTKTFTATVVLQLVGENRLKLDDYIEKWLPGVIQGNGYDGNKITIREILNHTSGIAEYSRS
KDVDFTDTKKSYTAEELVKMGISLPPDFAPGKGWSYSNTGYVLLGILIEKVTGNSYAEEVENR
IIEPLELSNTFLPGNSSVIPGTNHARGYVQPDGASELKDVTYYNPSAGSSAGDMISTADDLNK
FFSYLLGGKLLKEQQLKQMLTTVPTGKEGIDGYGLGIYETKLPSGVSIWGHSGGILGFTTLVG
GKLGGKHTLVVNWNSLGRTGSPNPFKNILLAEFSK;
SEQ ID No. 9:
NSSQEPNQKNRNGWKQVMQETIQIGAPGVLAKTSNKGKVNSYTAGVADLITKKPVKSDFRFRI
GSVTKTFTATTVLQLVGENRVQLDDPIEKWLPGLVQGNGYDGNQITIRQLLNHTSGIAEYLKS
KDADVMNSKKTYTAEEIVKIGLSLPPDFSPGKDWLYSNTGYVILGMLIEKITGNNYAEEIEKR
IIEPLDLPNTFLPGNSPVIPGKNHARGYVKMEETGELKDITYYNPSLANAAGDMISNADDLNK
FFSSLLGGKLLKERELKEMLTTVPVEGKGVGDGYGLGIYETKLPNGVSVWGHGGSIPGFMTFA
GGVIGGKHTFAVNVNSLGPVDILTQFDKMMQVEFNK.

13. A use of the D-stereospecific hydrolytic enzyme for deprotecting of at least one D-amino acid and/or D-amino acid derivative protected functional group of a substrate, preferably wherein the D-stereospecific hydrolytic enzyme is characterized by the following features:
at least one first 3₁₀-helix-2 structure comprising an amino acid sequence SXXK;
at least one first α-helix structure comprising an amino acids sequence YSN; and
at least one first β-sheet structure comprising an amino acid sequence HXG;
wherein X is an amino acid selected from the group consisting of proteinogenic amino acids;
wherein the first 3₁₀-helix-2 structure, the first α-helix structure and the first β-sheet structure are arranged in close proximity, further preferably the D-stereospecific hydrolytic enzyme having an amino acid sequence chosen from SEQ ID No. 1, No. 2, No. 3, No. 4, No. 5, No. 6, No. 7, No. 8, and No. 9:
SEQ ID No. 1:
ASLQLSQTKELLTIRIQTDAVTKYGKEDAAIDAYLKGEGFSGMALVAKDG
GVLLRKGYGFAGTNKLNRPDSKTRIASITKSFTAASIMQLVEQGKLSLTD
PVSKFVTGIPRGDDITIHMLLSHTSGLQSEFTRSGDVTIEQTIEELRTKQ
LKYEPGTTYLYSNNGYVLLAYVLEQLSGISYSDYVSEHFLTPLGMKNSGT
ATPATPTIQGYILQKNNEWAAAPYYVSQSGTGTLYSTVDDLLKWDTALRA
GKVVSEQSLEAMYTPHSDKNYGYGWIALGLNGEKGVFHNGSGSGYATGML
RNLDSGMTVILLGNHAGMDMTKLLQQVHKLAAEQ;
SEQ ID No. 2:
TIRIQTDAVTKYGKEDAAIDAYLKGEGFSGMALVAKDGEVLLRKGYGFSG
TNKLNHPDAKSRIASLTKSFTAASIMQLVEQGKLSLTDPVSKFVTGIPRG
DDITIHMLLSHTSGLPSEFTRSGNVTIEQTIAELRTKQLKYEPGTTYLYS
NNGYVLLAYVLEQLSGESYADYVSEHFLTPLGMKNSGTATPATATIQGYI
LQKNNEWAAAPYYVSQSGTGTLYSTVDDLLKWDQALRAGKVVSKQSLEAM
YTPHSDKNYGYGWIAINLNGEKGVFHNGSGSGYATGMLRNLDSGMTVILL
GNHAGMDMTKLLQQVHKLAAEQ;
SEQ ID No. 3:
ASLQLSQTKEFLTIRIQTDAVTKYGKEDAAIDAYLKGEGFSGMALVAKDG
EVLLRKGYGFSGTNKLNRPDAKSRIASITKSFTAASIMQLVEQGKLSLTD
PVSKFVTGIPRGDDITIHMLLSHTSGLPSEFTRSGDVTIEQTIAELRTKQ
LKYEPGTTYLYSNNGYVLLAYVLEQLAGESYADYVNEHELTPLGMKNSGT
ATPESPTIQGYILQKNNEWAAAPYYVSQSGTGTLYSTVDDLLKWDQALRA
GKVVSEQSLEAMYTPHSDKNYGYGWIAINLNGEKGVFHNGSGSGYATGML
RNLDSGMTVILLGNHAGMDMTKLLQQVHKLAAEQ;
SEQ ID No. 4:
SSVQTSTQRDRNSVKQAVRDTLQLGFPGILAKTSEGGKTWSYAAGVANLS
SKKPMKTDFRFRIGSVTKTFTATVVLQLAEENRLNLDDSIEKWLPGVIQG
NGYDDKQITIRQLLNHTSGIAEYTRSKSFDLMDTKKSYRAEELVKMGISM
PPDFAPGKSWSYSNTGYVLLGILIETVTGNSYAEEIENRIIEPLELSNTF
LPGNSSVIPGTKHARGYIQLDGASEPKDVTYYNPSMGSSAGDMISTADDL
NKFFSYLLGGKLLKEQQLKQMLTTVPTGEAALGRYGLGIYETKLPNGVSI
WGHGGSIPGFVTFAGGTLGGKHTLAVNLNSLNAESPDPFKNILLAEFSK;
SEQ ID No. 5:
SSLQTSTQSDRTSVKKAMRDELQLGYPGILAKISKGGKTWSYTAGVADLK
TKKPMKADFRFRIGSVTKTFIATVLLQLSGENRLNLDDSIEKWLPGVIQG
NGYDGNQITIRQILNHTSGIADYVNSKDFDIMDTKKSYTAEEFVKMGISL
PPDFAPGKGWSYSNTGYVLLGILIEKVTGNSYAEEVENRIIEPLDLSNTF
LPGNSSVIPGTKHARGYLQINGASELKDVTYINPGSSDGDMISTADDLNK
FFSCLLGGKLLKEQQLKQMLTTVPTNREGTGYGLGILEIKLPNGVSVWGH
RGAVPGFSTFAGGTLGGKHTLAINSNSLNLNNPEVFKNILLAEFRK;
SEQ ID No. 6:
SSLQTSTQSDRTSVKKAIRDELQLGYPGILAQISKGGKTWSYTAGIADLR
TKKPMKADFRFRIGSVTKTFIATVLLQLSGENRLNLDDSIEKWLPGVIQG
NGYDGNQITIRQILNHTSGIADYINSKDFDIMDTKKSYTAEEFVKMGISL
PPDFAPGKGWSYSNTGYVLLGILIEKVTGNSYAEEVENRIIEPLDLSNTF
LPGNSSVIPGTKHARGYLQLDGASELKDVTYINPGSSDGDMISTADDLNK
FFSYLLGGKLLKEQQLKQMLTTVPTNREGTGYGLGILEIKLPNGVSVWGH
RGGVLGFSTFAGGTLGGKHTLAINSNSFNINNPESFKNVLIAEFSK;
SEQ ID No. 7:
NSLQTSTQRDRNSVKEAMRDTLKLGYPGILAKTSEGGKTWSYAAGVADLS
NKKAMKTDERFRIGSVTKTFTATVVLQLAGENRLNLDDSIEKWLPGVIQG
NGYDDKQITIRQLLNHTSGIANYTRSKDFNMMDTKKSYTAEGLVKMGISM
PPDFAPGKSWSYSNTGYVLLGILIEKVTGNSYAEEIENRIIEPLELANTF
LPGNSSVIPGTKHARGYIQLDGASETKDVTYYNPSMGSSAGDMISTADDL
NKFFSYLLGGKLLKEQQLKQMLTTVPTGVGELGDSGLGIFKMKLSNGESI
WGHGGTIPGFLTFAGGTLGGKHTLAVNLNSLKADTPDPFKNILLAEFSK;
SEQ ID No. 8:
SSLQTNTQRDRTSVKQAMRDTLQLGYPGILAKTSEDGKTWGYAAGIADLR
TKKPMKTDFRFRIGSVTKTFTATVVLQLVGENRLKLDDYIEKWLPGVIQG
NGYDGNKITIREILNHTSGIAEYSRSKDVDFTDTKKSYTAEELVKMGISL
PPDFAPGKGWSYSNTGYVLLGILIEKVTGNSYAEEVENRIIEPLELSNTF
LPGNSSVIPGTNHARGYVQPDGASELKDVTYYNPSAGSSAGDMISTADDL
NKFFSYLLGGKLLKEQQLKQMLTTVPTGKEGIDGYGLGIYETKLPSGVSI
WGHSGGILGFTTLVGGKLGGKHTLVVNWNSLGRTGSPNPFKNILLAEFS
K;
SEQ ID No. 9:
NSSQEPNQKNRNGWKQVMQETIQIGAPGVLAKTSNKGKVNSYTAGVADLI
TKKPVKSDFRFRIGSVTKTFTATTVLQLVGENRVQLDDPIEKWLPGLVQG
NGYDGNQITIRQLLNHTSGIAEYLKSKDADVMNSKKTYTAEEIVKIGLSL
PPDFSPGKDWLYSNTGYVILGMLIEKITGNNYAEEIEKRIIEPLDLPNTF
LPGNSPVIPGKNHARGYVKMEETGELKDITYYNPSLANAAGDMISNADDL
NKFFSSLLGGKLLKERELKEMLTTVPVEGKGVGDGYGLGIYETKLPNGVS
VWGHGGSIPGFMTFAGGVIGGKHTFAVNVNSLGPVDILTQFDKMMQVEFN
K.

14. A method of manufacturing a carbohydrate; a carbohydrate derivative; a nucleotide; an oligonucleotide; a polynucleotide; a nucleotide derivative; an oligonucleotide derivative; a polynucleotide derivative; an amino acid; a peptide; an oligopeptide; a polypeptide; a protein; a peptide intermediate; a peptide derivative; an oligopeptide derivative; a polypeptide derivative: or mixtures thereof, the method comprising:
Providing a precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof,
at least one D-amino acid and/or D-amino acid derivative,
at least one first solvent,
optionally at least one second solvent,
optionally at least one third solvent,
a D-stereospecific hydrolytic enzyme, and
at least one first reagent;
Mixing the precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, the at least one first solvent and the at least one D-amino acid and/or D-amino acid derivative to obtain a first reaction mixture;
Reacting the precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof and the at least one D-amino acid and/or D-amino acid derivative to obtain a protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof;
Optionally removing the at least one first solvent from the first reaction mixture;
Optionally reacting the protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, in one or more further reaction steps with one or more further reagents to obtain a reacted protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof;
Mixing the protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, or the reacted protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, the at least one first reagent and optionally the at least one second solvent to obtain a second reaction mixture;
Reacting the protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, or the reacted protected precursor of the carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof and the at least one first reagent to obtain a protected carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof;
Optionally removing the at least one second solvent from the second reaction mixture;
Mixing the protected carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof, optionally the at least one third solvent and the D-stereospecific hydrolytic enzyme to obtain a third reaction mixture; and
Deprotecting the at least one D-amino acid and/or D-amino acid derivative from the protected carbohydrate; carbohydrate derivative; nucleotide; oligonucleotide; polynucleotide; nucleotide derivative; oligonucleotide derivative; polynucleotide derivative; amino acid; peptide; oligopeptide; polypeptide; protein; peptide intermediate; peptide derivative; oligopeptide derivative; polypeptide derivative; or mixtures thereof to obtain the carbohydrate; the carbohydrate derivative; the nucleotide; the oligonucleotide; polynucleotide; the nucleotide derivative; the oligonucleotide derivative; the polynucleotide derivative; the amino acid; the peptide; the oligopeptide; the polypeptide; the protein; the peptide intermediate; the peptide derivative; the oligopeptide derivative; the polypeptide derivative; or mixtures thereof.

15. Use of a D-amino acid and/or D-amino acid derivative as a protecting group for a functional group of a substrate.


SEQUENCE LISTING (193 sequences)

SEQ ID NO: 1 - protein, 334 aa, synthetic construct
ASLQLSQTKELLTIRIQTDAVTKYGKEDAAIDAYLKGEGFSGMALVAKDGGVLLRKGYGFAGTNKLNRPDSKTRIASITKSFTAASIMQLVEQGKLSLTDPVSKFVTGIPRGDDITIHMLLSHTSGLQSEFTRSGDVTIEQTIEELRTKQLKYEPGTTYLYSNNGYVLLAYVLEQLSGISYSDYVSEHFLTPLGMKNSGTATPATPTIQGYILQKNNEWAAAPYYVSQSGTGTLYSTVDDLLKWDTALRAGKVVSEQSLEAMYTPHSDKNYGYGWIALGLNGEKGVFHNGSGSGYATGMLRNLDSGMTVILLGNHAGMDMTKLLQQVHKLAAEQ

SEQ ID NO: 2 - protein, 322 aa, synthetic construct
TIRIQTDAVTKYGKEDAAIDAYLKGEGFSGMALVAKDGEVLLRKGYGFSGTNKLNHPDAKSRIASLTKSFTAASIMQLVEQGKLSLTDPVSKFVTGIPRGDDITIHMLLSHTSGLPSEFTRSGNVTIEQTIAELRTKQLKYEPGTTYLYSNNGYVLLAYVLEQLSGESYADYVSEHFLTPLGMKNSGTATPATATIQGYILQKNNEWAAAPYYVSQSGTGTLYSTVDDLLKWDQALRAGKVVSKQSLEAMYTPHSDKNYGYGWIAINLNGEKGVFHNGSGSGYATGMLRNLDSGMTVILLGNHAGMDMTKLLQQVHKLAAEQ

SEQ ID NO: 3 - protein, 334 aa, synthetic construct
ASLQLSQTKEFLTIRIQTDAVTKYGKEDAAIDAYLKGEGFSGMALVAKDGEVLLRKGYGFSGTNKLNRPDAKSRIASITKSFTAASIMQLVEQGKLSLTDPVSKFVTGIPRGDDITIHMLLSHTSGLPSEFTRSGDVTIEQTIAELRTKQLKYEPGTTYLYSNNGYVLLAYVLEQLAGESYADYVNEHFLTPLGMKNSGTATPESPTIQGYILQKNNEWAAAPYYVSQSGTGTLYSTVDDLLKWDQALRAGKVVSEQSLEAMYTPHSDKNYGYGWIAINLNGEKGVFHNGSGSGYATGMLRNLDSGMTVILLGNHAGMDMTKLLQQVHKLAAEQ

SEQ ID NO: 4 - protein, 349 aa, synthetic construct
SSVQTSTQRDRNSVKQAVRDTLQLGFPGILAKTSEGGKTWSYAAGVANLSSKKPMKTDFRFRIGSVTKTFTATVVLQLAEENRLNLDDSIEKWLPGVIQGNGYDDKQITIRQLLNHTSGIAEYTRSKSFDLMDTKKSYRAEELVKMGISMPPDFAPGKSWSYSNTGYVLLGILIETVTGNSYAEEIENRIIEPLELSNTFLPGNSSVIPGTKHARGYIQLDGASEPKDVTYYNPSMGSSAGDMISTADDLNKFFSYLLGGKLLKEQQLKQMLTTVPTGEAALGRYGLGIYETKLPNGVSIWGHGGSIPGFVTFAGGTLGGKHTLAVNLNSLNAESPDPFKNILLAEFSK

SEQ ID NO: 5 - protein, 346 aa, synthetic construct
SSLQTSTQSDRTSVKKAMRDELQLGYPGILAKISKGGKTWSYTAGVADLKTKKPMKADFRFRIGSVTKTFIATVLLQLSGENRLNLDDSIEKWLPGVIQGNGYDGNQITIRQILNHTSGIADYVNSKDFDIMDTKKSYTAEEFVKMGISLPPDFAPGKGWSYSNTGYVLLGILIEKVTGNSYAEEVENRIIEPLDLSNTFLPGNSSVIPGTKHARGYLQINGASELKDVTYINPGSSDGDMISTADDLNKFFSCLLGGKLLKEQQLKQMLTTVPTNREGTGYGLGILEIKLPNGVSVWGHRGAVPGFSTFAGGTLGGKHTLAINSNSLNLNNPEVFKNILLAEFRK

SEQ ID NO: 6 - protein, 346 aa, synthetic construct
SSLQTSTQSDRTSVKKAIRDELQLGYPGILAQISKGGKTWSYTAGIADLRTKKPMKADFRFRIGSVTKTFIATVLLQLSGENRLNLDDSIEKWLPGVIQGNGYDGNQITIRQILNHTSGIADYINSKDFDIMDTKKSYTAEEFVKMGISLPPDFAPGKGWSYSNTGYVLLGILIEKVTGNSYAEEVENRIIEPLDLSNTFLPGNSSVIPGTKHARGYLQLDGASELKDVTYINPGSSDGDMISTADDLNKFFSYLLGGKLLKEQQLKQMLTTVPTNREGTGYGLGILEIKLPNGVSVWGHRGGVLGFSTFAGGTLGGKHTLAINSNSFNINNPESFKNVLIAEFSK

SEQ ID NO: 7 - protein, 349 aa, synthetic construct
NSLQTSTQRDRNSVKEAMRDTLKLGYPGILAKTSEGGKTWSYAAGVADLSNKKAMKTDFRFRIGSVTKTFTATVVLQLAGENRLNLDDSIEKWLPGVIQGNGYDDKQITIRQLLNHTSGIANYTRSKDFNMMDTKKSYTAEGLVKMGISMPPDFAPGKSWSYSNTGYVLLGILIEKVTGNSYAEEIENRIIEPLELANTFLPGNSSVIPGTKHARGYIQLDGASETKDVTYYNPSMGSSAGDMISTADDLNKFFSYLLGGKLLKEQQLKQMLTTVPTGVGELGDSGLGIFKMKLSNGESIWGHGGTIPGFLTFAGGTLGGKHTLAVNLNSLKADTPDPFKNILLAEFSK

SEQ ID NO: 8 - protein, 350 aa, synthetic construct
SSLQTNTQRDRTSVKQAMRDTLQLGYPGILAKTSEDGKTWGYAAGIADLRTKKPMKTDFRFRIGSVTKTFTATVVLQLVGENRLKLDDYIEKWLPGVIQGNGYDGNKITIREILNHTSGIAEYSRSKDVDFTDTKKSYTAEELVKMGISLPPDFAPGKGWSYSNTGYVLLGILIEKVTGNSYAEEVENRIIEPLELSNTFLPGNSSVIPGTNHARGYVQPDGASELKDVTYYNPSAGSSAGDMISTADDLNKFFSYLLGGKLLKEQQLKQMLTTVPTGKEGIDGYGLGIYETKLPSGVSIWGHSGGILGFTTLVGGKLGGKHTLVVNWNSLGRTGSPNPFKNILLAEFSK

SEQ ID NO: 9 - protein, 351 aa, synthetic construct
NSSQEPNQKNRNGWKQVMQETIQIGAPGVLAKTSNKGKVNSYTAGVADLITKKPVKSDFRFRIGSVTKTFTATTVLQLVGENRVQLDDPIEKWLPGLVQGNGYDGNQITIRQLLNHTSGIAEYLKSKDADVMNSKKTYTAEEIVKIGLSLPPDFSPGKDWLYSNTGYVILGMLIEKITGNNYAEEIEKRIIEPLDLPNTFLPGNSPVIPGKNHARGYVKMEETGELKDITYYNPSLANAAGDMISNADDLNKFFSSLLGGKLLKERELKEMLTTVPVEGKGVGDGYGLGIYETKLPNGVSVWGHGGSIPGFMTFAGGVIGGKHTFAVNVNSLGPVDILTQFDKMMQVEFNK

SEQ ID NO: 10 - protein, 335 aa, synthetic construct
MASLQLSQTKELLTIRIQTDAVTKYGKEDAAIDAYLKGEGFSGMALVAKDGGVLLRKGYGFAGTNKLNRPDSKTRIASITKSFTAASIMQLVEQGKLSLTDPVSKFVTGIPRGDDITIHMLLSHTSGLQSEFTRSGDVTIEQTIEELRTKQLKYEPGTTYLYSNNGYVLLAYVLEQLSGISYSDYVSEHFLTPLGMKNSGTATPATPTIQGYILQKNNEWAAAPYYVSQSGTGTLYSTVDDLLKWDTALRAGKVVSEQSLEAMYTPHSDKNYGYGWIALGLNGEKGVFHNGSGSGYATGMLRNLDSGMTVILLGNHAGMDMTKLLQQVHKLAAEQ

SEQ ID NO: 11 - protein, 489 aa, synthetic construct
MTGIRGKKIAGWFNRHTVARGALIGIAAVSAFAGAGGAVAAAQPSSKVEAAQPASSKVIAVEVNGKAVEWNVQPLIKDGTTFVPLREAGKAAAGNIEWDGKTQTATIKVNGDVIVHRAGTSVVTVDGLAMNMSAPSLNIKGTLMLPLRSVTDALKASLQLSQTKELLTIRIQTDAVTKYGKEDAAIDAYLKGEGFSGMALVAKDGGVLLRKGYGFAGTNKLNRPDSKTRIASITKSFTAASIMQLVEQGKLSLTDPVSKFVTGIPRGDDITIHMLLSHTSGLQSEFTRSGDVTIEQTIEELRTKQLKYEPGTTYLYSNNGYVLLAYVLEQLSGISYSDYVSEHFLTPLGMKNSGTATPATPTIQGYILQKNNEWAAAPYYVSQSGTGTLYSTVDDLLKWDTALRAGKVVSEQSLEAMYTPHSDKNYGYGWIALGLNGEKGVFHNGSGSGYATGMLRNLDSGMTVILLGNHAGMDMTKLLQQVHKLAAEQ

SEQ ID NO: 12 - protein, 323 aa, synthetic construct
MTIRIQTDAVTKYGKEDAAIDAYLKGEGFSGMALVAKDGEVLLRKGYGFSGTNKLNHPDAKSRIASLTKSFTAASIMQLVEQGKLSLTDPVSKFVTGIPRGDDITIHMLLSHTSGLPSEFTRSGNVTIEQTIAELRTKQLKYEPGTTYLYSNNGYVLLAYVLEQLSGESYADYVSEHFLTPLGMKNSGTATPATATIQGYILQKNNEWAAAPYYVSQSGTGTLYSTVDDLLKWDQALRAGKVVSKQSLEAMYTPHSDKNYGYGWIAINLNGEKGVFHNGSGSGYATGMLRNLDSGMTVILLGNHAGMDMTKLLQQVHKLAAEQ

SEQ ID NO: 13 - protein, 519 aa, synthetic construct
MLNGFQVWQSCRFSFKRRRMRKRSWHGMTGIKGKKIAGWLNRHTVARSALIGIASVTAFAGVGGASVAAQTGSKGSKVEAVHPASSKAIAVEVNGKAVAWNVQPLIKDDTTFVPLREAGKAAAGNIEWDGKTKTATIKVNGDVIVHQAGTSVVTVDGLAMSMSAPSLNIKGTLMLPLRSVTDALKASLQLSQTKELLTIRIQTDAVTKYGKEDAAIDAYLKGEGFSGMALVAKDGEVLLRKGYGFSGTNKLNHPDAKSRIASLTKSFTAASIMQLVEQGKLSLTDPVSKFVTGIPRGDDITIHMLLSHTSGLPSEFTRSGNVTIEQTIAELRTKQLKYEPGTTYLYSNNGYVLLAYVLEQLSGESYADYVSEHFLTPLGMKNSGTATPATATIQGYILQKNNEWAAAPYYVSQSGTGTLYSTVDDLLKWDQALRAGKVVSKQSLEAMYTPHSDKNYGYGWIAINLNGEKGVFHNGSGSGYATGMLRNLDSGMTVILLGNHAGMDMTKLLQQVHKLAAEQ

SEQ ID NO: 14 - protein, 335 aa, synthetic construct
MASLQLSQTKEFLTIRIQTDAVTKYGKEDAAIDAYLKGEGFSGMALVAKDGEVLLRKGYGFSGTNKLNRPDAKSRIASITKSFTAASIMQLVEQGKLSLTDPVSKFVTGIPRGDDITIHMLLSHTSGLPSEFTRSGDVTIEQTIAELRTKQLKYEPGTTYLYSNNGYVLLAYVLEQLAGESYADYVNEHFLTPLGMKNSGTATPESPTIQGYILQKNNEWAAAPYYVSQSGTGTLYSTVDDLLKWDQALRAGKVVSEQSLEAMYTPHSDKNYGYGWIAINLNGEKGVFHNGSGSGYATGMLRNLDSGMTVILLGNHAGMDMTKLLQQVHKLAAEQ

SEQ ID NO: 15 - protein, 489 aa, synthetic construct
MTGIRGKKIAGWFNGHTVARSALIGMAAVTAFAGAGSAAAAAQPSSQVEAVQPASSKAIAVEVNGKAVVWNVQPLIKDGTTFVPLREAGKAAAGNIEWDGKTQTATIKVNGDVIVHPAGTSVITVDGLAMNMSAPSLNIKGTLMLPLRSVTDALKASLQLSQTKEFLTIRIQTDAVTKYGKEDAAIDAYLKGEGFSGMALVAKDGEVLLRKGYGFSGTNKLNRPDAKSRIASITKSFTAASIMQLVEQGKLSLTDPVSKFVTGIPRGDDITIHMLLSHTSGLPSEFTRSGDVTIEQTIAELRTKQLKYEPGTTYLYSNNGYVLLAYVLEQLAGESYADYVNEHFLTPLGMKNSGTATPESPTIQGYILQKNNEWAAAPYYVSQSGTGTLYSTVDDLLKWDQALRAGKVVSEQSLEAMYTPHSDKNYGYGWIAINLNGEKGVFHNGSGSGYATGMLRNLDSGMTVILLGNHAGMDMTKLLQQVHKLAAEQ

SEQ ID NO: 16 - protein, 350 aa, synthetic construct
MSSVQTSTQRDRNSVKQAVRDTLQLGFPGILAKTSEGGKTWSYAAGVANLSSKKPMKTDFRFRIGSVTKTFTATVVLQLAEENRLNLDDSIEKWLPGVIQGNGYDDKQITIRQLLNHTSGIAEYTRSKSFDLMDTKKSYRAEELVKMGISMPPDFAPGKSWSYSNTGYVLLGILIETVTGNSYAEEIENRIIEPLELSNTFLPGNSSVIPGTKHARGYIQLDGASEPKDVTYYNPSMGSSAGDMISTADDLNKFFSYLLGGKLLKEQQLKQMLTTVPTGEAALGRYGLGIYETKLPNGVSIWGHGGSIPGFVTFAGGTLGGKHTLAVNLNSLNAESPDPFKNILLAEFSK

SEQ ID NO: 17 - protein, 388 aa, synthetic construct
MKTRSQITCASLALLIAGSSLLYTTQTLIVKAEPTQSVSSSVQTSTQRDRNSVKQAVRDTLQLGFPGILAKTSEGGKTWSYAAGVANLSSKKPMKTDFRFRIGSVTKTFTATVVLQLAEENRLNLDDSIEKWLPGVIQGNGYDDKQITIRQLLNHTSGIAEYTRSKSFDLMDTKKSYRAEELVKMGISMPPDFAPGKSWSYSNTGYVLLGILIETVTGNSYAEEIENRIIEPLELSNTFLPGNSSVIPGTKHARGYIQLDGASEPKDVTYYNPSMGSSAGDMISTADDLNKFFSYLLGGKLLKEQQLKQMLTTVPTGEAALGRYGLGIYETKLPNGVSIWGHGGSIPGFVTFAGGTLGGKHTLAVNLNSLNAESPDPFKNILLAEFSK

SEQ ID NO: 18 - protein, 347 aa, synthetic construct
MSSLQTSTQSDRTSVKKAMRDELQLGYPGILAKISKGGKTWSYTAGVADLKTKKPMKADFRFRIGSVTKTFIATVLLQLSGENRLNLDDSIEKWLPGVIQGNGYDGNQITIRQILNHTSGIADYVNSKDFDIMDTKKSYTAEEFVKMGISLPPDFAPGKGWSYSNTGYVLLGILIEKVTGNSYAEEVENRIIEPLDLSNTFLPGNSSVIPGTKHARGYLQINGASELKDVTYINPGSSDGDMISTADDLNKFFSCLLGGKLLKEQQLKQMLTTVPTNREGTGYGLGILEIKLPNGVSVWGHRGAVPGFSTFAGGTLGGKHTLAINSNSLNLNNPEVFKNILLAEFRK

SEQ ID NO: 19 - protein, 384 aa, synthetic construct
KTRSKITCASLALLIGGSSLLYITPTSVVKAEPTQNVSSSLQTSTQSDRTSVKKAMRDELQLGYPGILAKISKGGKTWSYTAGVADLKTKKPMKADFRFRIGSVTKTFIATVLLQLSGENRLNLDDSIEKWLPGVIQGNGYDGNQITIRQILNHTSGIADYVNSKDFDIMDTKKSYTAEEFVKMGISLPPDFAPGKGWSYSNTGYVLLGILIEKVTGNSYAEEVENRIIEPLDLSNTFLPGNSSVIPGTKHARGYLQINGASELKDVTYINPGSSDGDMISTADDLNKFFSCLLGGKLLKEQQLKQMLTTVPTNREGTGYGLGILEIKLPNGVSVWGHRGAVPGFSTFAGGTLGGKHTLAINSNSLNLNNPEVFKNILLAEFRK

SEQ ID NO: 20 - protein, 347 aa, synthetic construct
MSSLQTSTQSDRTSVKKAIRDELQLGYPGILAQISKGGKTWSYTAGIADLRTKKPMKADFRFRIGSVTKTFIATVLLQLSGENRLNLDDSIEKWLPGVIQGNGYDGNQITIRQILNHTSGIADYINSKDFDIMDTKKSYTAEEFVKMGISLPPDFAPGKGWSYSNTGYVLLGILIEKVTGNSYAEEVENRIIEPLDLSNTFLPGNSSVIPGTKHARGYLQLDGASELKDVTYINPGSSDGDMISTADDLNKFFSYLLGGKLLKEQQLKQMLTTVPTNREGTGYGLGILEIKLPNGVSVWGHRGGVLGFSTFAGGTLGGKHTLAINSNSFNINNPESFKNVLIAEFSK

SEQ ID NO: 21 - protein, 385 aa, synthetic construct
MKTRSKITCASLALLIGGSSLLYITPTSVVKAEPTQNVSSSLQTSTQSDRTSVKKAIRDELQLGYPGILAQISKGGKTWSYTAGIADLRTKKPMKADFRFRIGSVTKTFIATVLLQLSGENRLNLDDSIEKWLPGVIQGNGYDGNQITIRQILNHTSGIADYINSKDFDIMDTKKSYTAEEFVKMGISLPPDFAPGKGWSYSNTGYVLLGILIEKVTGNSYAEEVENRIIEPLDLSNTFLPGNSSVIPGTKHARGYLQLDGASELKDVTYINPGSSDGDMISTADDLNKFFSYLLGGKLLKEQQLKQMLTTVPTNREGTGYGLGILEIKLPNGVSVWGHRGGVLGFSTFAGGTLGGKHTLAINSNSFNINNPESFKNVLIAEFSK

SEQ ID NO: 22 - protein, 350 aa, synthetic construct
MNSLQTSTQRDRNSVKEAMRDTLKLGYPGILAKTSEGGKTWSYAAGVADLSNKKAMKTDFRFRIGSVTKTFTATVVLQLAGENRLNLDDSIEKWLPGVIQGNGYDDKQITIRQLLNHTSGIANYTRSKDFNMMDTKKSYTAEGLVKMGISMPPDFAPGKSWSYSNTGYVLLGILIEKVTGNSYAEEIENRIIEPLELANTFLPGNSSVIPGTKHARGYIQLDGASETKDVTYYNPSMGSSAGDMISTADDLNKFFSYLLGGKLLKEQQLKQMLTTVPTGVGELGDSGLGIFKMKLSNGESIWGHGGTIPGFLTFAGGTLGGKHTLAVNLNSLKADTPDPFKNILLAEFSK

SEQ ID NO: 23 - protein, 389 aa, synthetic construct
MKKHSQITCTGLALLIIGGSLFYTTPTSSVKAESSIQNVSNSLQTSTQRDRNSVKEAMRDTLKLGYPGILAKTSEGGKTWSYAAGVADLSNKKAMKTDFRFRIGSVTKTFTATVVLQLAGENRLNLDDSIEKWLPGVIQGNGYDDKQITIRQLLNHTSGIANYTRSKDFNMMDTKKSYTAEGLVKMGISMPPDFAPGKSWSYSNTGYVLLGILIEKVTGNSYAEEIENRIIEPLELANTFLPGNSSVIPGTKHARGYIQLDGASETKDVTYYNPSMGSSAGDMISTADDLNKFFSYLLGGKLLKEQQLKQMLTTVPTGVGELGDSGLGIFKMKLSNGESIWGHGGTIPGFLTFAGGTLGGKHTLAVNLNSLKADTPDPFKNILLAEFSK

SEQ ID NO: 24 - protein, 351 aa, synthetic construct
MSSLQTNTQRDRTSVKQAMRDTLQLGYPGILAKTSEDGKTWGYAAGIADLRTKKPMKTDFRFRIGSVTKTFTATVVLQLVGENRLKLDDYIEKWLPGVIQGNGYDGNKITIREILNHTSGIAEYSRSKDVDFTDTKKSYTAEELVKMGISLPPDFAPGKGWSYSNTGYVLLGILIEKVTGNSYAEEVENRIIEPLELSNTFLPGNSSVIPGTNHARGYVQPDGASELKDVTYYNPSAGSSAGDMISTADDLNKFFSYLLGGKLLKEQQLKQMLTTVPTGKEGIDGYGLGIYETKLPSGVSIWGHSGGILGFTTLVGGKLGGKHTLVVNWNSLGRTGSPNPFKNILLAEFSK

SEQ ID NO: 25 - protein, 437 aa, synthetic construct
MAQIVGIDRYLCTGALIYMREGFSLSLVRKSGTVSKHLYNMNRGEECLMKIRSQITCASLALLIAGSSLLYTTPTSIVKAEPTQNVSSSLQTNTQRDRTSVKQAMRDTLQLGYPGILAKTSEDGKTWGYAAGIADLRTKKPMKTDFRFRIGSVTKTFTATVVLQLVGENRLKLDDYIEKWLPGVIQGNGYDGNKITIREILNHTSGIAEYSRSKDVDFTDTKKSYTAEELVKMGISLPPDFAPGKGWSYSNTGYVLLGILIEKVTGNSYAEEVENRIIEPLELSNTFLPGNSSVIPGTNHARGYVQPDGASELKDVTYYNPSAGSSAGDMISTADDLNKFFSYLLGGKLLKEQQLKQMLTTVPTGKEGIDGYGLGIYETKLPSGVSIWGHSGGILGFTTLVGGKLGGKHTLVVNWNSLGRTGSPNPFKNILLAEFSK

SEQ ID NO: 26 - protein, 352 aa, synthetic construct
MNSSQEPNQKNRNGWKQVMQETIQIGAPGVLAKTSNKGKVNSYTAGVADLITKKPVKSDFRFRIGSVTKTFTATTVLQLVGENRVQLDDPIEKWLPGLVQGNGYDGNQITIRQLLNHTSGIAEYLKSKDADVMNSKKTYTAEEIVKIGLSLPPDFSPGKDWLYSNTGYVILGMLIEKITGNNYAEEIEKRIIEPLDLPNTFLPGNSPVIPGKNHARGYVKMEETGELKDITYYNPSLANAAGDMISNADDLNKFFSSLLGGKLLKERELKEMLTTVPVEGKGVGDGYGLGIYETKLPNGVSVWGHGGSIPGFMTFAGGVIGGKHTFAVNVNSLGPVDILTQFDKMMQVEFNK

SEQ ID NO: 27 - protein, 388 aa, synthetic construct
MKTRNQIKLASLAVLLAGTTLSTPGFTVKAESTQNISNSSQEPNQKNRNGWKQVMQETIQIGAPGVLAKTSNKGKVNSYTAGVADLITKKPVKSDFRFRIGSVTKTFTATTVLQLVGENRVQLDDPIEKWLPGLVQGNGYDGNQITIRQLLNHTSGIAEYLKSKDADVMNSKKTYTAEEIVKIGLSLPPDFSPGKDWLYSNTGYVILGMLIEKITGNNYAEEIEKRIIEPLDLPNTFLPGNSPVIPGKNHARGYVKMEETGELKDITYYNPSLANAAGDMISNADDLNKFFSSLLGGKLLKERELKEMLTTVPVEGKGVGDGYGLGIYETKLPNGVSVWGHGGSIPGFMTFAGGVIGGKHTFAVNVNSLGPVDILTQFDKMMQVEFNK

SEQ ID NO: 28 - protein, 6 aa, synthetic construct
  MOD_RES 6: note = Tyrosine is 3-nitrotyrosine, i.e. modified by NO2
XAKAKY

SEQ ID NO: 29 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FCAKAKY

SEQ ID NO: 30 - protein, 7 aa, synthetic construct
  MOD_RES 1: note = phenylalanine is D-phenylalanine protected by a benzyloxycarbonyl group serving as a protecting group
  MOD_RES 7: note = Tyrosine is 3-nitrotyrosine, i.e. modified by NO2
FCAKAKY

SEQ ID NO: 31 - protein, 7 aa, synthetic construct
  MOD_RES 1: note = phenylalanine is D-phenylalanine protected by a succinic group serving as a protecting group
  MOD_RES 7: note = Tyrosine is 3-nitrotyrosine, i.e. modified by NO2
FCAKAKY

SEQ ID NO: 32 - protein, 7 aa, synthetic construct
  MOD_RES 1: note = phenylalanine is D-phenylalanine protected by an acetate group serving as a protecting group
  MOD_RES 7: note = Tyrosine is 3-nitrotyrosine, i.e. modified by NO2
FCAKAKY

SEQ ID NO: 33 - protein, 7 aa, synthetic construct
  MOD_RES 1: note = phenylalanine is D-phenylalanine protected by a tert-butyloxycarbonyl group serving as a protecting group
  MOD_RES 7: note = Tyrosine is 3-nitrotyrosine, i.e. modified by NO2
FCAKAKY

SEQ ID NO: 34 - protein, 7 aa, synthetic construct
  MOD_RES 1: note = phenylalanine is D-phenylalanine protected by a 2-aminobenzoic acid serving as a protecting group
  MOD_RES 7: note = Tyrosine is 3-nitrotyrosine, i.e. modified by NO2
FCAKAKY

SEQ ID NO: 35 - protein, 7 aa, synthetic construct
  MOD_RES 1: note = phenylalanine is D-phenylalanine protected by a fluorenylmethyloxycarbonyl group serving as a protecting group
  MOD_RES 7: note = Tyrosine is 3-nitrotyrosine, i.e. modified by NO2
FCAKAKY

SEQ ID NO: 36 - protein, 6 aa, synthetic construct
  MOD_RES 3: note = lysine is protected by D-phenylalanin which is protected by a benzoyl group
  MOD_RES 6: note = Tyrosine is 3-nitrotyrosine, i.e. modified by NO2
AAKAKY

SEQ ID NO: 37 - protein, 7 aa, synthetic construct
  MOD_RES 1: note = phenylalanine is D-phenylalanine
  MOD_RES 7: note = Tyrosine is 3-nitrotyrosine, i.e. modified by NO2
FCAKAKY

SEQ ID NO: 38 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FGAKAKY

SEQ ID NO: 39 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FAAKAKY

SEQ ID NO: 40 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FSAKAKY

SEQ ID NO: 41 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FTAKAKY

SEQ ID NO: 42 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FLAKAKY

SEQ ID NO: 43 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FVAKAKY

SEQ ID NO: 44 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FIAKAKY

SEQ ID NO: 45 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FFAKAKY

SEQ ID NO: 46 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FYAKAKY

SEQ ID NO: 47 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FWAKAKY

SEQ ID NO: 48 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FRAKAKY

SEQ ID NO: 49 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FKAKAKY

SEQ ID NO: 50 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FHAKAKY

SEQ ID NO: 51 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FDAKAKY

SEQ ID NO: 52 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FEAKAKY

SEQ ID NO: 53 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FQAKAKY

SEQ ID NO: 54 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FNAKAKY

SEQ ID NO: 55 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FMAKAKY

SEQ ID NO: 56 - protein, 7 aa, synthetic construct
  MOD_RES 1: note = phenylalanine is D-phenylalanine protected by a Benzoyl group serving as a protecting group
  MOD_RES 7: note = Tyrosine is 3-nitrotyrosine, i.e. modified by NO2
FPAKAKY

SEQ ID NO: 57 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AMFAAAK

SEQ ID NO: 58 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AFFAAAK

SEQ ID NO: 59 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AYFAAAK

SEQ ID NO: 60 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AWFAAAK

SEQ ID NO: 61 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AVFAAAK

SEQ ID NO: 62 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
ALFAAAK

SEQ ID NO: 63 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AIFAAAK

SEQ ID NO: 64 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AAFAAAK

SEQ ID NO: 65 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AGFAAAK

SEQ ID NO: 66 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
APFAAAK

SEQ ID NO: 67 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
ASFAAAK

SEQ ID NO: 68 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AEFAAAK

SEQ ID NO: 69 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
ADFAAAK

SEQ ID NO: 70 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AQFAAAK

SEQ ID NO: 71 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AKFAAAK

SEQ ID NO: 72 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
ARFAAAK

SEQ ID NO: 73 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AHFAAAK

SEQ ID NO: 74 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AAFMAAK

SEQ ID NO: 75 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AAFFAAK

SEQ ID NO: 76 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AAFVAAK

SEQ ID NO: 77 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AAFLAAK

SEQ ID NO: 78 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AAFAAAK

SEQ ID NO: 79 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AAFGAAK

SEQ ID NO: 80 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AAFPAAK

SEQ ID NO: 81 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AAFSAAK

SEQ ID NO: 82 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AAFNAAK

SEQ ID NO: 83 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AAFEAAK

SEQ ID NO: 84 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AAFRAAK

SEQ ID NO: 85 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AAFHAAK

SEQ ID NO: 86 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAFFAAK

SEQ ID NO: 87 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAWFAAK

SEQ ID NO: 88 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAYFAAK

SEQ ID NO: 89 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AALFAAK

SEQ ID NO: 90 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAMFAAK

SEQ ID NO: 91 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAHFAAK

SEQ ID NO: 92 - protein, 7 aa, synthetic construct
  MOD_RES 7: note = Lysine is modified by 2,4-dinitrophenol
  MOD_RES 1: note = Alanine is protected by a 2-aminobenzoic acid
AAFFAAK

SEQ ID NO: 93 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAFFAAK

SEQ ID NO: 94 - protein, 7 aa, synthetic construct
  MOD_RES 7: note = Lysine is modified by 2,4-dinitrophenol
  MOD_RES 1: note = Alanine is protected by a 2-aminobenzoic acid
AAWFAAK

SEQ ID NO: 95 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAWFAAK

SEQ ID NO: 96 - protein, 7 aa, synthetic construct
  MOD_RES 7: note = Lysine is modified by 2,4-dinitrophenol
  MOD_RES 1: note = Alanine is protected by a 2-aminobenzoic acid
AAYFAAK

SEQ ID NO: 97 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAYFAAK

SEQ ID NO: 98 - protein, 7 aa, synthetic construct
  MOD_RES 7: note = Lysine is modified by 2,4-dinitrophenol
  MOD_RES 1: note = Alanine is protected by a 2-aminobenzoic acid
AAAFAAK

SEQ ID NO: 99 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAAFAAK

SEQ ID NO: 100 - protein, 7 aa, synthetic construct
  MOD_RES 7: note = Lysine is modified by 2,4-dinitrophenol
  MOD_RES 1: note = Alanine is protected by a 2-aminobenzoic acid
AAVFAAK

SEQ ID NO: 101 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAVFAAK

SEQ ID NO: 102 - protein, 7 aa, synthetic construct
  MOD_RES 7: note = Lysine is modified by 2,4-dinitrophenol
  MOD_RES 1: note = Alanine is protected by a 2-aminobenzoic acid
AALFAAK

SEQ ID NO: 103 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AALFAAK

SEQ ID NO: 104 - protein, 7 aa, synthetic construct
  MOD_RES 7: note = Lysine is modified by 2,4-dinitrophenol
  MOD_RES 1: note = Alanine is protected by a 2-aminobenzoic acid
AASFAAK

SEQ ID NO: 105 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AASFAAK

SEQ ID NO: 106 - protein, 7 aa, synthetic construct
  MOD_RES 7: note = Lysine is modified by 2,4-dinitrophenol
  MOD_RES 1: note = Alanine is protected by a 2-aminobenzoic acid
AAMFAAK

SEQ ID NO: 107 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAMFAAK

SEQ ID NO: 108 - protein, 7 aa, synthetic construct
  MOD_RES 7: note = Lysine is modified by 2,4-dinitrophenol
  MOD_RES 1: note = Alanine is protected by a 2-aminobenzoic acid
AAHFAAK

SEQ ID NO: 109 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAHFAAK

SEQ ID NO: 110 - protein, 7 aa, synthetic construct
  MOD_RES 7: note = Lysine is modified by 2,4-dinitrophenol
  MOD_RES 1: note = Alanine is protected by a 2-aminobenzoic acid
AAKFAAK

SEQ ID NO: 111 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAKFAAK

SEQ ID NO: 112 - protein, 7 aa, synthetic construct
  MOD_RES 7: note = Lysine is modified by 2,4-dinitrophenol
  MOD_RES 1: note = Alanine is protected by a 2-aminobenzoic acid
AARFAAK

SEQ ID NO: 113 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AARFAAK

SEQ ID NO: 114 - protein, 7 aa, synthetic construct
  MOD_RES 7: note = Lysine is modified by 2,4-dinitrophenol
  MOD_RES 1: note = Alanine is protected by a 2-aminobenzoic acid
AAQFAAK

SEQ ID NO: 115 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAQFAAK

SEQ ID NO: 116 - protein, 7 aa, synthetic construct
  MOD_RES 7: note = Lysine is modified by 2,4-dinitrophenol
  MOD_RES 1: note = Alanine is protected by a 2-aminobenzoic acid
AAEFAAK

SEQ ID NO: 117 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAEFAAK

SEQ ID NO: 118 - protein, 7 aa, synthetic construct
  MOD_RES 7: note = Lysine is modified by 2,4-dinitrophenol
  MOD_RES 1: note = Alanine is protected by a 2-aminobenzoic acid
AADFAAK

SEQ ID NO: 119 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AADFAAK

SEQ ID NO: 120 - protein, 7 aa, synthetic construct
  MOD_RES 7: note = Lysine is modified by 2,4-dinitrophenol
  MOD_RES 1: note = Alanine is protected by a 2-aminobenzoic acid
AAPFAAK

SEQ ID NO: 121 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAPFAAK

SEQ ID NO: 122 - protein, 7 aa, synthetic construct
  MOD_RES 7: note = Lysine is modified by 2,4-dinitrophenol
  MOD_RES 1: note = Alanine is protected by a 2-aminobenzoic acid
AAGFAAK

SEQ ID NO: 123 - protein, 7 aa, synthetic construct
  MOD_RES 3: note = The amino acid is a D-amino acid
  MOD_RES 7: note = Lysine is modified by 2,4-dinitrophenol
  MOD_RES 1: note = Alanine is protected by a 2-aminobenzoic acid
AAGFAAK

SEQ ID NO: 124 - protein, 5 aa, synthetic construct
XAAAG

SEQ ID NO: 125 - protein, 5 aa, synthetic construct
  MOD_RES 1: note = The amino acid is D-amino acid
  MOD_RES 2: note = The amino acid is D-amino acid
  MOD_RES 3: note = The amino acid is D-amino acid
  MOD_RES 4: note = The amino acid is D-amino acid
XAAAG

SEQ ID NO: 126 - protein, 5 aa, synthetic construct
FAAAG

SEQ ID NO: 127 - protein, 5 aa, synthetic construct
  MOD_RES 1: note = The amino acid is a D-amino acid
  MOD_RES 2: note = The amino acid is a D-amino acid
  MOD_RES 3: note = The amino acid is a D-amino acid
  MOD_RES 4: note = The amino acid is a D-amino acid
FAAAG

SEQ ID NO: 128 - protein, 6 aa, synthetic construct
  MOD_RES 1: note = Glycine is protected by a Benzoyl group
GFAAAG

SEQ ID NO: 129 - protein, 6 aa, synthetic construct
  MOD_RES 1: note = Glycine is protected by a Benzoyl group
  MOD_RES 2: note = The amino acid is a D-amino acid
  MOD_RES 3: note = The amino acid is a D-amino acid
  MOD_RES 4: note = The amino acid is a D-amino acid
  MOD_RES 5: note = The amino acid is a D-amino acid
GFAAAG

SEQ ID NO: 130 - protein, 5 aa, synthetic construct
LAAAG

SEQ ID NO: 131 - protein, 5 aa, synthetic construct
  MOD_RES 1: note = The amino acid is a D-amino acid
  MOD_RES 2: note = The amino acid is a D-amino acid
  MOD_RES 3: note = The amino acid is a D-amino acid
  MOD_RES 4: note = The amino acid is a D-amino acid
LAAAG

SEQ ID NO: 132 - protein, 6 aa, synthetic construct
  MOD_RES 1: note = Glycine is protected by a Benzoyl group
GLAAAG

SEQ ID NO: 133 - protein, 6 aa, synthetic construct
  MOD_RES 1: note = Glycine is protected by a Benzoyl group
  MOD_RES 2: note = The amino acid is a D-amino acid
  MOD_RES 3: note = The amino acid is a D-amino acid
  MOD_RES 4: note = The amino acid is a D-amino acid
  MOD_RES 5: note = The amino acid is a D-amino acid
GLAAAG

SEQ ID NO: 134 - protein, 5 aa, synthetic construct
MAAAG

SEQ ID NO: 135 - protein, 5 aa, synthetic construct
  MOD_RES 1: note = The amino acid is a D-amino acid
  MOD_RES 2: note = The amino acid is a D-amino acid
  MOD_RES 3: note = The amino acid is a D-amino acid
  MOD_RES 4: note = The amino acid is a D-amino acid
MAAAG

SEQ ID NO: 136 - protein, 6 aa, synthetic construct
  MOD_RES 1: note = Glycine is protected by a Benzoyl group
GMAAAG

SEQ ID NO: 137 - protein, 6 aa, synthetic construct
  MOD_RES 1: note = Glycine is protected by a Benzoyl group
  MOD_RES 2: note = The amino acid is a D-amino acid
  MOD_RES 3: note = The amino acid is a D-amino acid
  MOD_RES 4: note = The amino acid is a D-amino acid
  MOD_RES 5: note = The amino acid is a D-amino acid
GMAAAG

SEQ ID NO: 138 - protein, 5 aa, synthetic construct
RAAAG

SEQ ID NO: 139 - protein, 5 aa, synthetic construct
  MOD_RES 1: note = The amino acid is a D-amino acid
  MOD_RES 2: note = The amino acid is a D-amino acid
  MOD_RES 3: note = The amino acid is a D-amino acid
  MOD_RES 4: note = The amino acid is a D-amino acid
RAAAG

SEQ ID NO: 140 - protein, 6 aa, synthetic construct
  MOD_RES 1: note = Glycine is protected by a Benzoyl group
GRAAAG

SEQ ID NO: 141 - protein, 6 aa, synthetic construct
  MOD_RES 1: note = Glycine is protected by a Benzoyl group
  MOD_RES 2: note = The amino acid is a D-amino acid
  MOD_RES 3: note = The amino acid is a D-amino acid
  MOD_RES 4: note = The amino acid is a D-amino acid
  MOD_RES 5: note = The amino acid is a D-amino acid
GRAAAG

SEQ ID NO: 142 - protein, 5 aa, synthetic construct
AAAAG

SEQ ID NO: 143 - protein, 5 aa, synthetic construct
  MOD_RES 1: note = The amino acid is a D-amino acid
  MOD_RES 2: note = The amino acid is a D-amino acid
  MOD_RES 3: note = The amino acid is a D-amino acid
  MOD_RES 4: note = The amino acid is a D-amino acid
AAAAG

SEQ ID NO: 144 - protein, 6 aa, synthetic construct
  MOD_RES 1: note = Glycine is protected by a Benzoyl group
GAAAAG

SEQ ID NO: 145 - protein, 6 aa, synthetic construct
  MOD_RES 1: note = Glycine is protected by a Benzoyl group
  MOD_RES 2: note = The amino acid is a D-amino acid
  MOD_RES 3: note = The amino acid is a D-amino acid
  MOD_RES 4: note = The amino acid is a D-amino acid
  MOD_RES 5: note = The amino acid is a D-amino acid
GAAAAG

SEQ ID NO: 146 - protein, 5 aa, synthetic construct
GAAAG

SEQ ID NO: 147 - protein, 5 aa, synthetic construct
  MOD_RES 1: note = The amino acid is a D-amino acid
  MOD_RES 2: note = The amino acid is a D-amino acid
  MOD_RES 3: note = The amino acid is a D-amino acid
  MOD_RES 4: note = The amino acid is a D-amino acid
GAAAG

SEQ ID NO: 148 - protein, 6 aa, synthetic construct
  MOD_RES 1: note = Glycine is protected by a Benzoyl group
GGAAAG

SEQ ID NO: 149 - protein, 6 aa, synthetic construct
  MOD_RES 1: note = Glycine is protected by a Benzoyl group
  MOD_RES 2: note = The amino acid is a D-amino acid
  MOD_RES 3: note = The amino acid is a D-amino acid
  MOD_RES 4: note = The amino acid is a D-amino acid
  MOD_RES 5: note = The amino acid is a D-amino acid
GGAAAG

SEQ ID NO: 150 - protein, 5 aa, synthetic construct
PAAAG

SEQ ID NO: 151 - protein, 5 aa, synthetic construct
  MOD_RES 1: note = The amino acid is a D-amino acid
  MOD_RES 2: note = The amino acid is a D-amino acid
  MOD_RES 3: note = The amino acid is a D-amino acid
  MOD_RES 4: note = The amino acid is a D-amino acid
PAAAG

SEQ ID NO: 152 - protein, 6 aa, synthetic construct
  MOD_RES 1: note = Glycine is protected by a Benzoyl group
GPAAAG

SEQ ID NO: 153 - protein, 6 aa, synthetic construct
  MOD_RES 1: note = Glycine is protected by a Benzoyl group
  MOD_RES 2: note = The amino acid is a D-amino acid
  MOD_RES 3: note = The amino acid is a D-amino acid
  MOD_RES 4: note = The amino acid is a D-amino acid
  MOD_RES 5: note = The amino acid is a D-amino acid
GPAAAG

SEQ ID NO: 154 - protein, 6 aa, synthetic construct
  MOD_RES 6: note = Tyrosine is 3-nitrotyrosine, i.e. modified by NO2
GAKAKY

SEQ ID NO: 155 - protein, 6 aa, synthetic construct
  MOD_RES 6: note = Tyrosine is 3-nitrotyrosine, i.e. modified by NO2
AAKAKY

SEQ ID NO: 156 - protein, 6 aa, synthetic construct
  MOD_RES 6: note = Tyrosine is 3-nitrotyrosine, i.e. modified by NO2
SAKAKY

SEQ ID NO: 157 - protein, 6 aa, synthetic construct
  MOD_RES 6: note = Tyrosine is 3-nitrotyrosine, i.e. modified by NO2
TAKAKY

SEQ ID NO: 158 - protein, 6 aa, synthetic construct
  MOD_RES 6: note = Tyrosine is 3-nitrotyrosine, i.e. modified by NO2
LAKAKY

SEQ ID NO: 159 - protein, 6 aa, synthetic construct
  MOD_RES 6: note = Tyrosine is 3-nitrotyrosine, i.e. modified by NO2
VAKAKY

SEQ ID NO: 160 - protein, 6 aa, synthetic construct
  MOD_RES 6: note = Tyrosine is 3-nitrotyrosine, i.e. modified by NO2
IAKAKY

SEQ ID NO: 161 - protein, 6 aa, synthetic construct
  MOD_RES 6: note = Tyrosine is 3-nitrotyrosine, i.e. modified by NO2
FAKAKY

SEQ ID NO: 162 - protein, 6 aa, synthetic construct
  MOD_RES 6: note = Tyrosine is 3-nitrotyrosine, i.e. modified by NO2
YAKAKY

SEQ ID NO: 163 - protein, 6 aa, synthetic construct
  MOD_RES 6: note = Tyrosine is 3-nitrotyrosine, i.e. modified by NO2
WAKAKY

SEQ ID NO: 164 - protein, 6 aa, synthetic construct
  MOD_RES 6: note = Tyrosine is 3-nitrotyrosine, i.e. modified by NO2
RAKAKY

SEQ ID NO: 165 - protein, 6 aa, synthetic construct
  MOD_RES 6: note = Tyrosine is 3-nitrotyrosine, i.e. modified by NO2
KAKAKY

SEQ ID NO: 166 - protein, 6 aa, synthetic construct
  MOD_RES 6: note = Tyrosine is 3-nitrotyrosine, i.e. modified by NO2
HAKAKY

SEQ ID NO: 167 - protein, 6 aa, synthetic construct
  MOD_RES 6: note = Tyrosine is 3-nitrotyrosine, i.e. modified by NO2
DAKAKY

SEQ ID NO: 168 - protein, 6 aa, synthetic construct
  MOD_RES 6: note = Tyrosine is 3-nitrotyrosine, i.e. modified by NO2
EAKAKY

SEQ ID NO: 169 - protein, 6 aa, synthetic construct
  MOD_RES 6: note = Tyrosine is 3-nitrotyrosine, i.e. modified by NO2
QAKAKY

SEQ ID NO: 170 - protein, 6 aa, synthetic construct
  MOD_RES 6: note = Tyrosine is 3-nitrotyrosine, i.e. modified by NO2
NAKAKY

SEQ ID NO: 171 - protein, 6 aa, synthetic construct
  MOD_RES 6: note = Tyrosine is 3-nitrotyrosine, i.e. modified by NO2
MAKAKY

SEQ ID NO: 172 - protein, 6 aa, synthetic construct
  MOD_RES 6: note = Tyrosine is 3-nitrotyrosine, i.e. modified by NO2
PAKAKY

SEQ ID NO: 173 - protein, 4 aa, synthetic construct
  MOD_RES 4: note = Lysine modified by 2,4-dinitrophenol
FAAK

SEQ ID NO: 174 - protein, 4 aa, synthetic construct
  MOD_RES 4: note = Lysine modified by 2,4-dinitrophenol
AAAK

SEQ ID NO: 175 - protein, 4 aa, synthetic construct
  MOD_RES 4: note = Lysine modified by 2,4-dinitrophenol
MAAK

SEQ ID NO: 176 - protein, 4 aa, synthetic construct
  MOD_RES 4: note = Lysine modified by 2,4-dinitrophenol
FAAK

SEQ ID NO: 177 - protein, 4 aa, synthetic construct
  MOD_RES 4: note = Lysine modified by 2,4-dinitrophenol
VAAK

SEQ ID NO: 178 - protein, 4 aa, synthetic construct
  MOD_RES 4: note = Lysine modified by 2,4-dinitrophenol
LAAK

SEQ ID NO: 179 - protein, 4 aa, synthetic construct
  MOD_RES 4: note = Lysine modified by 2,4-dinitrophenol
AAAK

SEQ ID NO: 180 - protein, 4 aa, synthetic construct
  MOD_RES 4: note = Lysine modified by 2,4-dinitrophenol
GAAK

SEQ ID NO: 181 - protein, 4 aa, synthetic construct
  MOD_RES 4: note = Lysine modified by 2,4-dinitrophenol
PAAK

SEQ ID NO: 182 - protein, 4 aa, synthetic construct
  MOD_RES 4: note = Lysine modified by 2,4-dinitrophenol
SAAK

SEQ ID NO: 183 - protein, 4 aa, synthetic construct
  MOD_RES 4: note = Lysine modified by 2,4-dinitrophenol
NAAK

SEQ ID NO: 184 - protein, 4 aa, synthetic construct
  MOD_RES 4: note = Lysine modified by 2,4-dinitrophenol
EAAK

SEQ ID NO: 185 - protein, 4 aa, synthetic construct
  MOD_RES 4: note = Lysine modified by 2,4-dinitrophenol
RAAK

SEQ ID NO: 186 - protein, 4 aa, synthetic construct
  MOD_RES 4: note = Lysine modified by 2,4-dinitrophenol
HAAK

SEQ ID NO: 187 - protein, 6 aa, synthetic construct
  MOD_RES 6: note = Tyrosine is 3-nitrotyrosine, i.e. modified by NO2
CAKAKY

SEQ ID NO: 188 - protein, 6 aa, synthetic construct
  MOD_RES 1: note = Glycine is protected by a Benzoyl group
GXAAAG

SEQ ID NO: 189 - protein, 6 aa, synthetic construct
  MOD_RES 1: note = Glycine is protected by a Benzoyl group
  MOD_RES 2: note = The amino acid is a D-amino acid
  MOD_RES 3: note = The amino acid is D-amino acid
  MOD_RES 4: note = The amino acid is D-amino acid
  MOD_RES 5: note = The amino acid is D-amino acid
GXAAAG

SEQ ID NO: 190 - protein, 7 aa, synthetic construct
  MOD_RES 1: note = Alanine is protected by a 2-aminobenzoic acid
  MOD_RES 7: note = Lysine is modified by 2,4-dinitrophenol
AAXFAAK

SEQ ID NO: 191 - protein, 7 aa, synthetic construct
  MOD_RES 1: note = Alanine is protected by a 2-aminobenzoic acid
  MOD_RES 3: note = The amino acid is a D-amino acid
  MOD_RES 7: note = Lysine is modified by 2,4-dinitrophenol
AAXFAAK

SEQ ID NO: 192 - protein, 7 aa, synthetic construct
  MOD_RES 1: note = Alanine is protected by a 2-aminobenzoic acid
  MOD_RES 7: note = Lysine is modified by 2,4-dinitrophenol
  MOD_RES 3: note = The amino acid is a D-amino acid
AXFAAAK

SEQ ID NO: 193 - protein, 7 aa, synthetic construct
  MOD_RES 1: note = Alanine is protected by a 2-aminobenzoic acid
  MOD_RES 2: note = The amino acid is a D-amino acid
  MOD_RES 3: note = The amino acid is a D-amino acid
  MOD_RES 7: note = Lysine is modified by 2,4-dinitrophenol
AXFAAAK